(19) United States
(12) Patent Application Publication
(10) Pub. No.: US 2024/0102005 A1
(43) Pub. Date: Mar. 28, 2024

(54) METHODS AND SYSTEMS FOR ENGINEERING ANTIBODIES, AND ANTIGEN-BINDING FRAGMENTS THEREOF, TO HAVE ALTERED CHARACTERISTICS

(71) Applicant: 10x Genomics, Inc., Pleasanton, CA (US)

(72) Inventors: Michael John Terry Stubbington, Counterslip Bristol (GB); Wyatt James McDonnell, Pleasanton, CA (US); Katherine Pfeiffer, Pleasanton, CA (US)

(21) Appl. No.: 18/525,142

(22) Filed: Nov. 30, 2023

Related U.S. Application Data
(63) Continuation of application No. PCT/US2022/031649, filed on May 31, 2022.
(60) Provisional application No. 63/195,643, filed on Jun. 1, 2021.

(51) Int. Cl. C12N 15/10

(52) U.S. Cl. CPC C12N 15/1086

(57) ABSTRACT

The present disclosure generally relates to methods and systems for engineering antibodies, and antigen-binding fragments thereof, to have altered characteristics. The present disclosure also provides a high-throughput method useful for identifying the engineered antibodies, or antigen-binding fragments thereof, that acquired the altered characteristics having performed the methods. These methods and systems have implications, for example, in the rapid development of biotherapeutics having desired and/or improved, e.g., affinity, specificity and/or activity-related, characteristics.

### CROSS-REFERENCE TO RELATED APPLICATIONS

This application claims the benefit of U.S. Provisional Patent Application No. 63/195,643, filed Jun. 1, 2021, which is incorporated herein by reference in its entirety.

### FIELD

The present disclosure relates to methods and systems for engineering antibodies, and antigen-binding fragments thereof, to have altered characteristics.

### BACKGROUND

The ability to engineer antibodies, and antigen-binding fragments thereof, to have altered characteristics, e.g., altered affinity, specificity or activity, is important to the development of improved biotherapeutics. Existing approaches to engineering antibodies, or antigen-binding fragments thereof, can include screening to identify a lead antibody, optimizing the lead antibody, and validating the optimized leads. These types of approaches can be time-consuming, taking place over months to years. Further, any biotherapeutic developed from a lead antibody not derived from a human source, or not derived from a product of natural human immune selection and maturation, may ultimately provoke an immune response in a recipient of the biotherapeutic.

The disclosure provided herein satisfies a need for methods and systems that reliably and rapidly generate and identify immunotherapeutic molecules, such as antibodies and antigen-binding fragments thereof, that have altered characteristics, such as improved safety and/or activity.

### SUMMARY

Provided herein are, inter alia, a method for engineering an antigen-binding site of an antibody, or antigen-binding fragment thereof, a method for preparing a library of variant antibodies, or variant antigen-binding fragments thereof, and a system for engineering an antigen-binding site of an antibody, and antigen-binding fragment thereof, to have an improved characteristic.

In one aspect, the description provides for a method of engineering an antigen-binding site of an antibody, or antigen-binding fragment thereof, to have an altered characteristic. In the method, a nucleic acid sequence encoding a selected antibody, or selected antigen-binding fragment thereof, is provided. The selected antibody, or selected antigen-binding fragment thereof, binds a target antigen. The nucleic acid sequence is amplified in an error-prone amplification reaction to produce a plurality of polynucleotides encoding variant antibodies, or variant antigen-binding fragments thereof. The plurality of variant antibodies, or variant antigen-binding fragments thereof, is expressed in a plurality of cells. A cell of the plurality of cells expresses a variant antibody, or variant antigen-binding fragment thereof, of the plurality of variant antibodies, or variant antigen-binding fragments thereof. The plurality of cells is incubated in a reaction mixture that further includes the target antigen. The target antigen is coupled to a reporter oligonucleotide. The reaction mixture is partitioned into a plurality of partitions and a partition of the plurality of partitions includes: (i) a partitioned cell bound to the target antigen, and (ii) a plurality of nucleic acid barcode molecules including a partition-specific barcode sequence. In the partition, barcoded nucleic acid molecules are generated. The barcoded nucleic acid molecules include: (i) a first barcoded nucleic acid molecule including a sequence of the reporter oligonucleotide or a reverse complement thereof and the partition-specific barcode sequence or reverse complement thereof, and (ii) a second barcoded nucleic acid molecule including a nucleic acid sequence encoding the variant antibody, or variant antigen-binding fragment thereof, expressed by the cell or reverse complement thereof and the partition-specific barcode sequence or reverse complement thereof. The variant antibody, or variant antigen-binding fragment thereof is identified as engineered to comprise the altered characteristic based on the generated first barcoded nucleic acid molecule.

In another aspect, the description provides for a method of preparing a library of variant antibodies, or variant antigen-binding fragments thereof, having an altered characteristic or characteristics. In the method, a nucleic acid sequence encoding a selected antibody, or selected antigen-binding fragment thereof, is provided. The selected antibody, or selected antigen-binding fragment thereof, binds a target antigen. The nucleic acid sequence is amplified in an error-prone amplification reaction to produce a plurality of polynucleotides encoding variant antibodies, or variant antigen-binding fragments thereof. The plurality of variant antibodies, or variant antigen-binding fragments thereof, is expressed in a plurality of cells. A cell of the plurality of cells expresses a variant antibody, or variant antigen-binding fragment thereof, of the plurality of variant antibodies, or variant antigen-binding fragments thereof. The plurality of cells is incubated in a reaction mixture that further includes the target antigen. The target antigen is coupled to a reporter oligonucleotide. The reaction mixture is partitioned into a plurality of partitions and a partition of the plurality of partitions includes: (i) a partitioned cell bound to the target antigen, and (ii) a plurality of nucleic acid barcode molecules including a partition-specific barcode sequence. In the partition, barcoded nucleic acid molecules are generated. The barcoded nucleic acid molecules include: (i) a first barcoded nucleic acid molecule including a sequence of the reporter oligonucleotide or a reverse complement thereof and the partition-specific barcode sequence or reverse complement thereof, and (ii) a second barcoded nucleic acid molecule including a nucleic acid sequence encoding the variant antibody, or variant antigen-binding fragment thereof, expressed by the cell or reverse complement thereof and the partition-specific barcode sequence or reverse complement thereof. The variant antibody, or variant antigen-binding fragment thereof is identified for inclusion in the library based on the generated first barcoded nucleic acid molecule.

In yet another aspect, the disclosure provides for a method of engineering an antigen-binding site of an antibody, or an antigen-binding fragment thereof, to have an altered characteristic. In the method, a nucleic acid sequence encoding a selected antibody derived from nucleic acids of a cell of a human donor, or selected antigen-binding fragment thereof, is provided. The selected human antibody, or selected antigen-binding fragment thereof, binds a target antigen. The nucleic acid sequence is amplified in an error-prone amplification reaction to produce a plurality of polynucleotides encoding variant antibodies, or variant antigen-binding fragments thereof. The plurality of variant antibodies, or variant antigen-binding fragments thereof, is expressed in a plurality of mammalian cells, wherein a mammalian cell of the plurality of mammalian cells expresses a variant antibody, or variant antigen-binding fragment thereof, of the plurality of variant antibodies, or variant antigen-binding fragments thereof. The variant antibody, or variant antigen-binding fragment thereof, is identified as engineered to have the altered characteristic.

In a further aspect, the disclosure provides a method of preparing a library of variant antibodies, or variant antigen-binding fragments thereof, that have an altered characteristic or characteristics. In the method, a nucleic acid sequence encoding a selected antibody derived from nucleic acids of a cell of a human donor, or selected antigen-binding fragment thereof, is provided. The selected human antibody, or selected antigen-binding fragment thereof, binds a target antigen. The nucleic acid sequence is amplified in an error-prone amplification reaction to produce a plurality of polynucleotides encoding variant antibodies, or variant antigen-binding fragments thereof. The plurality of variant antibodies, or variant antigen-binding fragments thereof, is expressed in a plurality of mammalian cells, wherein a mammalian cell of the plurality of mammalian cells expresses a variant antibody, or variant antigen-binding fragment thereof, of the plurality of variant antibodies, or variant antigen-binding fragments thereof. The variant antibody, or variant antigen-binding fragment thereof, is identified for inclusion in the library if it has an altered characteristic or characteristics.

In an additional aspect, the disclosure provides a system for engineering an antigen-binding site of an antibody, or antigen-binding fragment thereof, to have an improved characteristic. The system includes: (i) reagents for performing error-prone amplification, (ii) a target antigen coupled to a reporter oligonucleotide, (iii) a plurality of nucleic acid barcode molecules comprising a partition-specific barcode sequence, and (iv) reagents for generating a plurality barcoded nucleic acid molecules formed by complementary base pairing of (a) a capture sequence of the plurality of nucleic acid barcode molecules and (b) a capture handle sequence of the first reporter oligonucleotide and/or a capture handle sequence of a polynucleotide encoding an engineered variant of the antibody or antigen-binding fragment thereof.

The foregoing is merely a summary and is illustrative only. It is not intended to be in any way limiting. In addition to the illustrative embodiments and features described herein, further aspects, embodiments, objects and features of the disclosure will become fully apparent from the drawings and the detailed description and the claims.

### BRIEF DESCRIPTION OF THE DRAWINGS

FIG. 1 shows an exemplary microfluidic channel structure for partitioning individual biological particles in accordance with some embodiments of the disclosure.

FIG. 2 shows an exemplary microfluidic channel structure for the controlled partitioning of beads into discrete droplets.

FIG. 3 shows an exemplary microfluidic channel structure for delivering barcode carrying beads to droplets.

FIG. 4 shows an exemplary barcode carrying bead.

FIG. 5 illustrates another example of a barcode carrying bead.

FIG. 6 schematically illustrates an example microwell array.

FIG. 7 schematically illustrates an example workflow for processing nucleic acid molecules.

FIG. 8 schematically illustrates examples of labelling agents.

FIG. 9 depicts an example of a barcode carrying bead.

FIGS. 10A, 10B and 10C schematically depict an example workflow for processing nucleic acid molecules.

FIG. 11 depicts a block diagram illustrating an example of a computing system, in accordance with some example embodiments.

FIG. 12 illustrates a nested PCR scheme for enriching, from a sample of nucleic acid sequences, those that encode at least an antigen-binding fragment of an antibody.

### DETAILED DESCRIPTION OF THE DISCLOSURE

The present disclosure generally relates to, inter alia, methods of engineering an antigen-binding site of an antibody, or antigen-binding fragment thereof, to have an altered characteristic; methods of preparing a library of variant antibodies, or antigen-binding fragments thereof, that have an altered characteristic; and systems for engineering an antigen-binding site of an antibody, or antigen-binding site thereof, to have an improved characteristic.

In the following detailed description, reference is made to the accompanying drawings, which form a part of the disclosure hereof. In the drawings, similar symbols generally identify similar components, unless context dictates otherwise. The illustrative alternatives described in the detailed description, drawings, and claims are not meant to be limiting. Other alternatives may be used and other changes may be made without departing from the spirit or scope of the subject matter presented here. It will be readily understood that the aspects, as generally described herein, and illustrated in the Figures, can be arranged, substituted, combined, and designed in a wide variety of different configurations, all of which are explicitly contemplated and make part of this application.

Unless otherwise defined, all terms of art, notations, and other scientific terms or terminology used herein are intended to have the meanings commonly understood by those of skill in the art to which this application pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art.

The practice of the present invention will employ, unless otherwise indicated, conventional techniques of molecular biology, microbiology, cell biology, biochemistry, nucleic acid chemistry, and immunology, which are well known to those skilled in the art. Such techniques are explained fully in the literature, such as Sambrook, J., & Russell, D. W. (2012). Molecular Cloning: A Laboratory Manual (4th ed.). Cold Spring Harbor, NY: Cold Spring Harbor Laboratory and Sambrook, J., & Russel, D. W. (2001). Molecular Cloning: A Laboratory Manual (3rd ed.). Cold Spring Harbor, NY: Cold Spring Harbor Laboratory (jointly referred to herein as “Sambrook”); Ausubel, F. M. (1987). Current Protocols in Molecular Biology. New York, NY: Wiley (including supplements through 2014); Bollag, D. M. et al. (1996). Protein Methods. New York, NY: Wiley-Liss; Huang, L. et al. (2005). Nonviral Vectors for Gene Therapy. San Diego: Academic Press; Kaplitt, M. G. et al. (1995). Viral Vectors: Gene Therapy and Neuroscience Applications. San Diego, CA: Academic Press; Lefkovits, I. (1997). The Immunology Methods Manual: The Comprehensive Sourcebook of Techniques. San Diego, CA: Academic Press; Doyle, A. et al. (1998). Cell and Tissue Culture: Laboratory Procedures in Biotechnology. New York, NY: Wiley; Mullis, K. B., Ferre, F. & Gibbs, R. (1994). PCR: The Polymerase Chain Reaction. Boston: Birkhauser Publisher; Greenfield, E. A. (2014). Antibodies: A Laboratory Manual (2nd ed.). New York, NY: Cold Spring Harbor Laboratory Press; Beaucage, S. L. et al. (2000). Current Protocols in Nucleic Acid Chemistry. New York, NY: Wiley, (including supplements through 2014); and Makrides, S. C. (2003). Gene Transfer and Expression in Mammalian Cells. Amsterdam, NL: Elsevier Sciences B.V., the disclosures of which are incorporated herein by reference.

Unless otherwise defined, all terms of art, notations and other scientific terms or terminology used herein are intended to have the meanings commonly understood by those of skill in the art to which this disclosure pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art.

The singular form “a”, “an”, and “the” include plural references unless the context clearly dictates otherwise. For example, the term “a cell” includes one or more cells, including mixtures thereof. “A and/or B” is used herein to include all of the following alternatives: “A”, “B”, “A or B”, and “A and B”.

As used herein, “isolated” may refer to molecules, e.g., antigen-binding molecules such as antibodies or antigen-binding fragments thereof, polypeptides, polynucleotides or vectors, that are at least partially free of other biological molecules from the cells or cell culture from which they are produced. Such biological molecules include nucleic acids, proteins, other antibodies or antigen-binding fragments, lipids, carbohydrates, or other material such as cellular debris and growth medium. An isolated antibody or antigen-binding fragment may further be at least partially free of expression system components such as biological molecules from a host cell or of the growth medium thereof. Generally, the term “isolated” is not intended to refer to a complete absence of such biological molecules or to an absence of water, buffers, or salts or to components of a pharmaceutical formulation that includes the antibodies or antigen-binding fragments.

As used herein, a “subject” or an “individual” or a “donor” includes animals, such as human (e.g., human individuals) and non-human animals. The term “non-human animals” includes all vertebrates, e.g., mammals, e.g., rodents, e.g., mice, non-human primates, and other mammals, such as e.g., rat, mouse, cat, dog, cow, pig, sheep, horse, goat, rabbit; and non-mammals, such as amphibians, reptiles, etc. A subject can be a healthy individual, an asymptomatic individual, an individual that has or is suspected of having a disease (e.g., cancer or infection), an individual having a pre-disposition to a disease, an individual that is in need of therapy for a disease, an individual who has recovered from a disease, or an individual that is resistant to a disease. In any event, the subject may have been exposed to an antigen characteristic of the disease, such as an antigen capable of producing an antibody immune response associated with the disease.

The term “barcode” is used herein to refer to a label, or identifier, that conveys or is capable of conveying information (e.g., information about an analyte in a sample, a bead, and/or a nucleic acid barcode molecule). A barcode can be part of an analyte or nucleic acid barcode molecule, or independent of an analyte or nucleic acid barcode molecule. A barcode can be attached to an analyte or nucleic acid barcode molecule in a reversible or irreversible manner. A particular barcode can be unique relative to other barcodes. Barcodes can have a variety of different formats. For example, barcodes can include polynucleotide barcodes, random nucleic acid and/or amino acid sequences, and synthetic nucleic acid and/or amino acid sequences. A barcode can be attached to an analyte or to another moiety or structure in a reversible or irreversible manner. A barcode can be added to, for example, a fragment of a deoxyribonucleic acid (DNA) or ribonucleic acid (RNA) sample before or during sequencing of the sample.

Barcodes can allow for or facilitate identification and/or quantification of individual sequencing-reads. In some embodiments, a barcode can be configured for use as a fluorescent barcode. For example, in some embodiments, a barcode can be configured for hybridization to fluorescently labeled oligonucleotide probes. Barcodes can be configured to spatially resolve molecular components found in biological samples, for example, at single-cell resolution (e.g., a barcode can be or can include a “spatial barcode”). In some embodiments, a barcode includes two or more sub-barcodes that together function as a single barcode. For example, a polynucleotide barcode can include two or more polynucleotide sequences (e.g., sub-barcodes). In some embodiments, the two or more sub-barcodes are separated by one or more non-barcode sequences. In some embodiments, the two or more sub-barcodes are not separated by non-barcode sequences.

In some embodiments, a barcode can include one or more unique molecular identifiers (UMIs). Generally, a unique molecular identifier is a contiguous nucleic acid segment or two or more non-contiguous nucleic acid segments that function as a label or identifier for a particular analyte, or for a nucleic acid barcode molecule that binds a particular analyte (e.g., mRNA) via the capture sequence.

A UMI can include one or more specific polynucleotides sequences, one or more random nucleic acid and/or amino acid sequences, and/or one or more synthetic nucleic acid and/or amino acid sequences. In some embodiments, the UMI is a nucleic acid sequence that does not substantially hybridize to analyte nucleic acid molecules in a biological sample. In some embodiments, the UMI has less than 80% sequence identity (e.g., less than 70%, 60%, 50%, or less than 40% sequence identity) to the nucleic acid sequences across a substantial part (e.g., 80% or more) of the nucleic acid molecules in the biological sample. These nucleotides can be completely contiguous, i.e., in a single stretch of adjacent nucleotides, or they can be separated into two or more separate subsequences that are separated by 1 or more nucleotides.

The term “biological particle” may be used herein to generally refer to a discrete biological system derived from a biological sample. The biological particle may be a macromolecule. The biological particle may be a small molecule. The biological particle may be a virus. The biological particle may be a cell or derivative of a cell. The biological particle may be an organelle. The biological particle may be a nucleus of a cell. The biological particle may be a rare cell from a population of cells. The biological particle may be any type of cell, including without limitation prokaryotic cells, eukaryotic cells, bacterial, fungal, plant, mammalian, or other animal cell type, mycoplasmas, normal tissue cells, tumor cells, or any other cell type, whether derived from single cell or multicellular organisms. The biological particle may be a constituent of a cell. The biological particle may be or may include DNA, RNA, organelles, proteins, or any combination thereof. The biological particle may be or may include a matrix (e.g., a gel or polymer matrix) comprising a cell or one or more constituents from a cell (e.g., cell bead), such as DNA, RNA, organelles, proteins, or any combination thereof, from the cell. The biological particle may be obtained from a tissue of a subject. The biological particle may be a hardened cell. Such hardened cell may or may not include a cell wall or cell membrane. The biological particle may include one or more constituents of a cell, but may not include other constituents of the cell. An example of such constituents is a nucleus or an organelle. A cell may be a live cell. The live cell may be capable of being cultured, for example, being cultured when enclosed in a gel or polymer matrix, or cultured when comprising a gel or polymer matrix.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, the use of these terms in the specification does not by itself connote any required priority, precedence, or order.

It is understood that aspects and embodiments of the disclosure described herein include “comprising”, “consisting”, and “consisting essentially of” aspects and embodiments. As used herein, “comprising” is synonymous with “including”, “containing”, or “characterized by”, and is inclusive or open-ended and does not exclude additional, unrecited elements or method steps. As used herein, “consisting of” excludes any elements, steps, or ingredients not specified in the claimed composition or method. As used herein, “consisting essentially of” does not exclude materials or steps that do not materially affect the basic and novel characteristics of the claimed composition or method. Any recitation herein of the term “comprising”, particularly in a description of components of a composition or in a description of steps of a method, is understood to encompass those compositions and methods consisting essentially of and consisting of the recited components or steps.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

### Methods of the Disclosure

As described in more detail below, aspects of the disclosure relate to new approaches and methods of engineering an antigen-binding site of an antibody, or antigen-binding fragment thereof. Furthermore, other aspects of the disclosure relate to new approaches and methods of preparing a library of variant antibodies, or variant antigen-binding fragments thereof, that include an altered characteristic or altered characteristics.

### Providing a Nucleic Acid Sequence

Methods of the disclosure, in any of the aspects provided herein, include a step of providing a nucleic acid sequence encoding a selected antibody, or selected antigen-binding fragment thereof. The selected antibody, or selected antigen-binding fragment thereof, encoded by the provided nucleic acid sequence may be a “full antibody”, as is typically expressed by most mammals including humans, e.g., an immunoglobulin (Ig) molecule including four polypeptide chains, two heavy chains (HCs) and two light chains (LCs) inter-connected by disulfide bonds, or a multimer thereof (e.g. IgM). The selected antibody may be an IgA (e.g., IgA1 or IgA2), IgD, IgE, IgG (e.g., IgG1, IgG2, IgG3 and IgG4) or IgM antibody.

The selected antibody, or selected antigen-binding fragment thereof, (e.g., antigen-binding fragment of the selected antibody) encoded by the provided nucleic acid sequence, may be the antigen-binding fragment of the selected antibody. If it is the antigen-binding fragment of the selected antibody, then it may be any naturally occurring, enzymatically obtainable, synthetic, or genetically engineered polypeptide or glycoprotein derivative of the selected antibody. An antigen-binding fragment of the selected antibody may be any of a: (i) Fab fragment; (ii) F(ab′)2 fragment; (iii) Fd fragment; (iv) Fv fragment; (v) single-chain Fv (scFv) molecule; (vi) sdAb fragment; or (vii) minimal recognition unit consisting of the amino acid residues that mimic the hypervariable region of the selected antibody (e.g., an isolated complementarity determining region (CDR) such as a CDR3 peptide), or a constrained FWR3-CDR3-FWR4 peptide. Further, the antigen-binding fragment of the selected antibody may be an engineered molecule, such as a domain-specific antibody, single domain antibody, chimeric antibody, CDR-grafted antibody, diabody, triabody, tetrabody, minibody, nanobody (e.g., monovalent nanobodies, bivalent nanobodies, etc.), a small modular immunopharmaceutical (SMIP), or a shark immunoglobulin new antigen receptor (IgNAR) variable domain.

Further, if the selected antibody, or selected antigen-binding fragment thereof, encoded by the provided nucleic acid sequence is an antigen-binding fragment of the selected antibody, the antigen-binding fragment of the selected antibody may be a monomeric VH or VL domain of the selected antibody, or a configuration of a variable domain of the selected antibody with one or more constant domains, including any of a: (i) VH-CH1; (ii) VH-CH2; (iii) VH-CH3; (iv) VH-CH1-CH2; (v) VH-CH1-CH2-CH3; (vi) VH-CH2-CH3; (vii) VH-CL; (viii) VL-CH1; (ix) VL-CH2; (x) VL-CH3; (xi) VL-CH1-CH2; (xii) VL-CH1-CH2-CH3; (xiii) VL-CH2-CH3; and (xiv) VL-CL. In any configuration of variable and constant domains, the variable and constant domains can be either directly linked to one another or may be linked by a full or partial hinge or linker region. A hinge region may consist of at least 2 (e.g., 5, 10, 15, 20, 40, 60 or more) amino acids, which result in a flexible or semi-flexible linkage between adjacent variable and/or constant domains in a single polypeptide molecule. Furthermore, any of the selected antibodies or selected antigen-binding fragments thereof can be mono-specific or multi-specific (e.g., bi-specific).

In some embodiments, the selected antibody, or selected antigen-binding fragment thereof, is a human antibody. In some embodiments, the selected antibody, or selected antigen-binding fragment thereof, is an antigen-binding fragment of a human antibody. In other embodiments, the selected antibody, or selected antigen-binding fragment thereof, is a humanized antibody. In yet other embodiments, the selected antibody, or selected antigen-binding fragment thereof, is an antigen-binding fragment of a humanized antibody. In further still embodiments, the selected antibody, or selected antigen-binding fragment thereof, is a mouse antibody or is an antigen-binding fragment of a mouse antibody. In other embodiments, the selected antibody, or selected antigen binding fragment thereof may be an antibody or antigen-binding fragment of a rabbit, goat, rat, sheep, horse, cow, llama, alpaca, a camel, chicken or shark antibody.

The selected antibody, or selected antigen-binding fragment thereof, may be selected due to its ability to bind a target antigen. The target antigen to which the selected antibody, or antigen-binding fragment thereof, may bind may be any antigen for which the development and identification of variants of the selected antibody, or selected antigen-binding fragment thereof, is desirable. The target antigen may be an antigen associated with a pathogen or infectious agent, such as a viral, bacterial, parasitic, protozoal or prion agent. If the target antigen is associated with an infectious agent that is a viral agent, the viral agent may be an influenza virus, a coronavirus, a retrovirus, a rhinovirus, or a sarcoma virus. The viral agent may be severe acute respiratory syndrome coronavirus 1 (SARS-CoV-1), a SARS-CoV-2, a Middle East respiratory syndrome coronavirus (MERS-CoV)), or human immunodeficiency virus (HIV), influenza, respiratory syncytial virus, or Ebola virus. If the target antigen is associated with an infectious agent that is a viral agent, the target antigen may be corona virus spike (S) protein, an influenza hemagglutinin protein, an HIV envelope protein or any other a viral glycoprotein. Further, the target antigen may be associated with a tumor or a cancer. If the target agent is associated with a tumor or cancer, it may be, for example, a growth factor or a growth factor receptor. Examples of target antigens that may be associated with tumors or cancers include epidermal growth factor receptor (EGFR), CD38, platelet-derived growth factor receptor (PDGFR) alpha, insulin growth factor receptor (IGFR), CD20, CD19, CD47, or human epidermal growth factor receptor 2 (HER2). Alternatively, the target antigen may be an immune checkpoint molecule that may or may not be associated with tumors or cancers (e.g., CD38, PD-1, CTLA-4, TIGIT, LAG-3, VISTA, TIM-3), or it may be a cytokine, a GPCR, a cell-based co-stimulatory molecule, a cell-based co-inhibitory molecule or an ion channel. Other examples of a target antigen include autoantigens, virus-like particles and lipoparticles. Further still, the target antigen may be associated with a degenerative condition or disease (e.g., an amyloid protein or a tau protein).

The selected antibody, or selected antigen-binding fragment thereof, may be selected due to its binding a fragment of the target antigen that includes a region of interest, e.g., target antigen region of interest. The target antigen region of interest may be a particular domain, domains, epitope or epitopes of the target antigen. The target antigen region of interest may be a fragment of the target antigen that is a 10-200, 20-200, a 20-180, a 20-160, a 20-140, a 20-120, a 20-100, a 20-80, a 20-60, a 20-40, a 40-200, a 40-180, a 40-160, a 40-140, a 40-120, a 40-100, a 40-80, a 40-60, 60-200, a 60-180, a 60-160, a 60-140, a 60-120, a 60-100, a 60-80, a 80-200, a 80-180, a 80-160, a 80-140, a 80-120, a 80-100, a 100-200, a 150-100, or a 25-175 amino acid residue peptide region of the target antigen. The fragment of the target antigen may have an amino acid length that is 95%, 90%, 85%, 80%, 75%, 70%, 65%, 60%, 55%, 50%, 45%, 40%, 35%, 30%, 25%, 20%, 15%, 10%, or 5% that of the target antigen. The region of interest of the target antigen may include or may be an epitope of the target antigen, e.g., a linear or conformational or cryptic epitope. The region of interest of the target antigen may include or may be a domain of the target antigen, e.g., a unit or portion the antigen that is self-stabilizing and folds independently of the remainder of the antigen (and which can be determined by, for example, Hydrophobicity/Kyte-Doolittle plots, InterPro or PROSITE (www.ebi.ac.uk/interpro/) or protein BLAST).

The selected antibody, or selected antigen-binding fragment thereof, may be selected due to its binding the target antigen at a region of interest that includes one or more epitopes or domains of the target antigen that are involved in a signaling pathway, that interact with other proteins or peptides, or that result in or prevent a conformational change in the target antigen.

The provided nucleic acid sequence encoding the selected antibody, or selected antigen-binding fragment thereof, that binds the target antigen as described herein may be provided as a nucleic acid sequence of any type, e.g., DNA/cDNA molecule, suitable for amplification. The provided nucleic acid sequence encoding the selected antibody, or selected antigen-binding fragment thereof, may be provided as a plurality, or library, of nucleic acid sequences, e.g., DNA/cDNA molecules. The provided nucleic acid sequence encoding the selected antibody, or selected antigen-binding fragment thereof, may be provided as a nucleic acid sequence, e.g., DNA/cDNA, molecule cloned into a vector. The provided nucleic acid sequence encoding the selected antibody, or selected antigen-binding fragment thereof, may be provided as a plurality of nucleic acid sequences, e.g., DNA/cDNA molecules, cloned into a plurality of vectors. The provided nucleic acid sequence encoding the selected antibody, or selected antigen-binding fragment thereof, may be configured in such a way as to facilitate amplification by any method, e.g., including rolling circle amplification.

The provided nucleic acid sequence encoding the selected antibody, or selected antigen-binding fragment thereof, may have been derived, (or, alternatively, may have been obtained, discovered or sequenced), from a cell of a vertebrate. For example, the provided nucleic acid sequence encoding the selected antibody, or selected antigen-binding fragment thereof, may be derived from a cell or a sample of cells of a mammal, reptile, or fish. If the selected antibody, or selected antigen-binding fragment thereof, is derived from a cell of a mammal, it may be derived from a cell or a sample or cells of a human, a mouse, a rabbit, a goat, a rat, a sheep, a horse, a cow, a llama, an alpaca or a camel. If the selected antibody, or selected antigen-binding fragment thereof, is derived from a cell, or a sample of cells of the mouse, the rat, the rabbit, the chicken or the cow, then the mouse, rat, rabbit, chicken or cow may be transgenic and may be transgenic to express human antibodies. If the selected antibody, or selected antigen-binding fragment thereof, is derived from a cell of a fish, it may be derived from a cell or a sample of cells of a jawed fish, such as a shark. The cell or the sample of cells of the vertebrate from which the nucleic acid sequence encoding the selected antibody, or selected antigen-binding fragment thereof, may be derived may have been a cell of the B cell lineage, or of a sample of cells that includes cells of B cell lineage, e.g., memory B cells.

The cell, or sample of cells, from which the provided nucleic acid sequence encoding the selected antibody, or selected antigen-binding fragment thereof is derived may be a cell obtained from a donor, or a sample of cells obtained from a donor, such as a human donor. A cell or a sample of cells from which the provided nucleic acid sequence encoding the selected antibody, or selected antigen-binding fragment thereof, is derived may be a cell or a sample of cells obtained from a mouse, a transgenic mouse, a rat, a transgenic rat, a rabbit, a transgenic rabbit, a goat, a sheep, a horse, a cow, a transgenic cow, a llama, an alpaca, a camel, a chicken, a transgenic chicken, or a shark. If the cell is of a sample of cells, the sample may be a blood sample, a peripheral blood mononuclear cell sample or a plasma sample. By way of example, the sample of cells may be a blood sample of a human donor or a mouse, e.g., a transgenic mouse. The sample of cells may be a peripheral blood mononuclear cell sample of a human donor or a mouse, e.g., a transgenic mouse. The sample of cells may be a plasma sample of a human or a mouse, e.g., a transgenic mouse.

The cells or the sample of cells from which the provided nucleic acid sequence encoding the selected antibody, or selected antigen-binding fragment thereof, is derived need not be derived from a cell or a cell sample of a single donor. The cells or the sample of cells may be obtained by combining cells, or samples of cells, of multiple donors, e.g., cells or cell samples of more than one human, cells or cell samples of more than one mouse, cells or cell samples of more than one rat, cells or cell samples of more than one rabbit, cells or cell samples of more than one goat, cells or cell samples of more than one sheep, cells or cell samples of more than one horse, cells or cell samples of more than one cow, cells or cell samples of more than one llama, cells or cell samples of more than one alpaca, cells or cell samples of more than one camel or cells or cell samples of more than one shark. If the cells or the samples of cells are of multiple donors, the cells or samples of cells may be any combination of one or more of blood samples, peripheral blood mononuclear cell samples or plasma samples. If the cells or the samples of cells are of multiple donors, the cells or samples of cells may be any combination of one or more of human blood samples, human peripheral blood mononuclear cell samples or human plasma samples. If the cells or the samples of cells are of multiple donors, the cells or samples of cells may be any combination of one or more of mouse blood samples, mouse peripheral blood mononuclear cell samples or mouse plasma samples. If the cells or the samples of cells are of multiple donors, the cells or samples of cells may be any combination of one or more of transgenic mouse blood samples, transgenic mouse peripheral blood mononuclear cell samples or transgenic mouse plasma samples.

The donor, from which the cells or the sample of cells is obtained, may be known to have been exposed to the target antigen. Alternatively, the donor, from which the cells or the sample of cells is obtained, may be a donor suspected of having been exposed to the target antigen. The donor, from which the cells or the sample of cells is obtained, may be known or expected to be resistant to a pathogen or infectious agent that bears the target antigen.

In some embodiments disclosed herein, a secreted antibody is captured, e.g., on a surface of the cell from which the antibody is secreted, in a partition (e.g., containing a bead such as a gel bead, magnetic bead or paramagnetic bead) containing the cell, and/or in a matrix surrounding (e.g., encapsulating) the cell, e.g., a cell bead, for subsequent single cell analysis of the cell and the antibody molecules that it secretes. In some embodiments, a method disclosed herein is used for screening single cells in a heterogeneous population of cells, such as plasma cells in a subject.

In some embodiments herein, the method comprises contacting a cell, such as an isolated cell of B-cell lineage (e.g., a plasma cell), with a reporter agent, comprising a reporter nucleic acid molecule. The reporter agent may be one or more reporter barcoded second antibody binding agents (e.g., antigens), wherein a reporter barcode sequence, e.g., reporter sequence, is a unique oligonucleotide that can be amplified downstream. In some embodiments, the labelled cell comprises a complex coupled to a surface of the cell, and wherein the complex comprises (i) a capture agent, (ii) a secreted antibody or antigen binding fragment thereof, and (iii) the reporter agent.

In some embodiments, the capture agent is configured to couple to a cell surface molecule. In some embodiments, the cell surface molecule is a cell surface protein, e.g., a cell surface receptor such as CD19, CD20, CD45, or CD22. In some embodiments, the capture agent is configured to couple to the secreted antibody or antigen binding fragment thereof. For example, the capture agent may comprise one or more immunoglobulin (e.g., antibody) molecules (e.g., IgA, IgE, IgG, or IgM molecules) or any fragments (e.g., epitope-binding fragments) or derivatives thereof, and in any suitable combination. In some embodiments, the capture agent may be configured to couple to both the cell surface protein and the secreted antibody or antigen binding fragment thereof. In some examples, the capture agent is a first antibody binding agent. For example, a capture agent may be a multi-specific antibody, e.g., a bispecific antibody capable of binding to a cell surface molecule and to the secreted antibody or antigen binding fragment thereof.

In some embodiments, the capture agent may be tethered to a lipid, enabling insertion of the biomolecule into a cellular membrane where it could then tether the secreted antibody to the surface of the cell. In some embodiments, the capture agent may be configured to couple to both the cell surface protein and the secreted antibody or antigen binding fragment thereof. For example, a capture agent may be tethered to a lipid and comprise an antibody capable of binding to the secreted antibody or antigen binding fragment thereof.

It is to be understood that the nucleic acid sequence encoding the selected antibody, or selected antigen-binding site thereof, need not have been derived from a donor, a cell obtained from a donor or a cell sample of a donor. The nucleic acid sequence encoding the selected antibody, or selected antigen-binding site thereof, may have been derived from any source including a sequence of an antibody purchased from a vendor, a therapeutic antibody known in the art, a sequence having been selected from a phage or yeast scFv library, or a sequence synthesized based on a published disclosure.

### Error-Prone Amplification

In the methods provided herein the provided nucleic acid sequence or sequences encoding the selected antibody, or selected antigen-binding fragment thereof, are amplified in an error-prone amplification reaction. The error-prone amplification reaction may be conducted to introduce errors in the sequence encoding the selected antibody, or antigen-binding fragment thereof, resulting in one or more amino acid alterations, e.g., substitutions, insertions or deletions in the amino acid sequence of the selected antibody, or selected antigen-binding fragment thereof. The error-prone amplification reaction may also, or alternatively, result in an alteration that truncates the amino acid sequence of the selected antibody, or selected antigen-binding fragment thereof. The error-prone amplification may introduce errors resulting one, at least one, two, at least two, three, at least three, four, at least four, five, at least five, six, at least six, seven, at least seven, eight, at least eight, nine, at least nine, ten, at least ten, at most five, at most ten, at most fifteen, or at most twenty amino acid residue alterations in the selected antibody, or antigen-binding fragment thereof. The error-prone amplification may introduce errors resulting one to twenty, one to fifteen, one to ten, one to five, five to ten, five to fifteen, five to twenty, or five to fifteen amino acid residue alterations in the selected antibody, or antigen-binding fragment thereof. The error-prone amplification may introduce errors at a rate of 1-50 mutations/kilobase, 1-40 mutations/kilobase, 1-30 mutations/kilobase, 1-20, 10-50 mutations/kilobase mutations/kilobase, 20-50 mutations/kilobase, 30-50 mutations/kilobase or 20-40 mutations/kilobase in the provided nucleic acid sequence encoding the selected antibody, or selected antigen-binding fragment thereof. The introduction of the amino acid alterations in the selected antibody, or selected antigen-binding fragment thereof produces variant antibodies or variant antigen-binding fragments thereof, e.g., a plurality of variant antibodies or variant antigen-binding fragments thereof.

Error-prone amplification can be performed under any set of conditions well known in the art to increase the likelihood of introduction of errors during an amplification reaction. Conditions that increase the likelihood of introduction of errors during an amplification reaction, e.g., conditions conducive to error-prone amplification, include decreased quantity of template nucleic acid, imbalanced dNTP ratios, increased MgCl2 concentration, inclusion of MnCl2 and utilization of a polymerase without (or with diminished) proofreading capabilities, during the amplification reaction. If the likelihood of introduction of errors during an amplification reaction is to be increased is via decreased quantity of the template nucleic acid, the template nucleic acid may be present in the reaction at a total quantity of 500-1000, 550-1000, 600-1000, 650-1000, 700-1000, 750-1000, 800-1000, 850-1000, 900-1000, 950-1000, 500-550, 550-600, 600-650, 650-700, 700-750, 750-800, 800-850, 850-900, 900-950 nanograms. If the likelihood of introduction of errors during an amplification reaction is to be increased via decreased quantity of the template nucleic acid, the template nucleic acid present in the reaction may be a total quantity of 100-500, 150-500, 200-500, 250-500, 300-500, 350-500, 400-500, 450-500, 100-150, 150-200, 200-250, 250-300, 300-350, 350-400, 400-450, 450-500 nanograms. Alternatively, the likelihood of introduction of errors during an amplification reaction is to be increased is via decreased quantity of the template nucleic acid, the template nucleic acid present in the reaction may be a total quantity of 0.1 to 100, 1-100, 10-100, 20-100, 30-100, 40-100, 50-100, 60-100, 70-100, 80-100, 90-100, 0.1-10, 0.1-20, 0.1-30, 0.1-40, 0.1-50, 0.1-60, 0.1-70, 0.1-80, 0.1-90, 0.1-0.5, 0.1-0.4, 0.1-0.3, 0.1-0.2 nanograms.

If the likelihood of introduction of errors during an amplification reaction is to be increased is via “unbalanced dNTP concentration” in the amplification reaction, the “unbalanced dNTP concentration” may be one in which more dCTP and dTTP than dATP and dGTP is present in the amplification reaction. For example, dATP and dGTP may be included at 0.2-5, 1-5, 2-5-, 3-5, 4-5, 0.2-0.3, 0.3-0.4, 0.4-0.5, 0.5-0.6, 0.6-0.7, 0.7-0.8, 0.8-1 mM in the reaction, while dCTP and dTTP may be included at 0.2-25, 1-25, 5-25, 10-25, 15-25, 20-25, 0.2-0.3, 0.3-0.4, 0.4-0.5, 0.5-0.6, 0.6-0.7, 0.7-0.8, 0.8-1 mM in the reaction.

If the error-prone amplification reaction is performed to increase the likelihood of introduction of errors via increasing MgCl2 concentration, the MgCl2 concentration may be increased to 1-25, 1-20, 1-15, 1-10, 1-5, 1, 2, 3, 4, 5, 6, 7, 8, 9, 10, 11, 12, 13, 14, 15, 16, 17, 18, 19, 20, 21, 22, 23, 24, 25 mM. If the error-prone amplification reaction is performed to increase the likelihood of introduction of errors via including MnCl2 in the reaction, MnCl2 may be included in the reaction at a concentration of 10-200, 20-200, 30-200, 40-200, 50-200, 60-200, 70-200, 80-200, 90-200, 100-200, 125-200, 125-200, 150-200, 175-200, 10-20, 10-30, 10-40, 10-50, 10-60, 10-70, 10-80, 10-90, 10-100, 10-110, 10-120, 10-130, 10-140, 10-150, 10-160, 10-170, 10-180, 10-190 mM.

If the likelihood of introduction of errors during an amplification reaction is to be increased due to the inclusion of an error-prone polymerase, the error-prone polymerase may be a polymerase lacking, or with diminished, proof-reading capability. Examples of such polymerases include Mutazyme™ I and Mutazyme™ II (Agilent) or Taq DNA polymerase (NEB).

Kits for performing error-prone amplification are also commercially available to those of skill in the art and include PickMutant™ Error Prone PCR Kit (Canvax), JBS Error-Prone Kit (Jena Bioscience GmBH), GeneMorph II Random Mutagenesis Kit (Agilent).

In an embodiment, the error-prone amplification may be performed by rolling circle amplification. The rolling circle amplification may be conducted in a reaction mixture comprising a DNA polymerase, template DNA, random hexamers (for reaction priming), dNTPs, and a buffer containing MnCl2, wherein the MnCl2 concentration is responsible for increasing likelihood of introducing errors. The MnCl2 may be included in the reaction at 1-15, 1-10, 1-5, 1, 2, 3, 5, 6, 7, 8, 9, 10, 11, 12, 13, 14, 15 mM, with higher concentrations increasing likelihood of introducing errors. Additional details of error-prone amplification by rolling circle amplification can be found in Fujii et al., Nature Protocols 5(2006):2493-2497, incorporated herein by reference. Error-prone amplification via rolling circle amplification may also be adjusted to increasingly introduce errors in a nucleic acid sequence, or sequences, encoding (or originating from nucleic acid sequences encoding) a selected antibody, or selected antigen binding fragment thereof, by increasing the number of rounds of amplification.

### Expressing the Plurality of Variant Antibodies or Variant Antigen Binding Fragments Thereof

In any of the methods provided herein, the error-prone amplification produces a plurality of polynucleotides encoding variant antibodies, or variant antigen-binding fragments thereof. The plurality of variant antibodies, or variant antigen-binding fragments thereof, may be expressed in a plurality of cells. The expression may be such that a cell of the plurality of cells expresses a variant antibody, or variant antigen-binding fragment thereof, of the plurality of variant antibodies, or variant antigen-binding fragments thereof.

The expression of the variant antibodies, or variant antigen-binding fragments thereof, may be in any suitable cell for antibody expression. The cell may be a bacterial cell, e.g., E. coli cell, a yeast cell, a eukaryotic cell, a mammalian cell or an insect cell. Non-limiting examples of cells that may express the variant antibody, or variant antigen-binding fragment thereof, include: a baby hamster kidney (BHK) cell, a Chinese hamster ovary cell (CHO cell), an African green monkey kidney cell (Vero cell), a human A549 cell, a human cervix cell, a human CHME5 cell, a human PER.C6 cell, a NS0 murine myeloma cell, a human epidermoid larynx cell, a human fibroblast cell, a human HEK-293 cell, a human HeLa cell, a human HepG2 cell, a human HUH-7 cell, a human MRC-5 cell, a human muscle cell, a mouse 3T3 cell, a mouse connective tissue cell, a mouse muscle cell, a rabbit kidney cell, a Pichia pastoris cell or a Saccharomyces cerevisiae cell. These cells, and others suitable for expression of a variant antibody, or variant antigen-binding fragment thereof, are available from many sources, including the American Type Culture Collection (Manassas, VA).

The variant antibodies, or variant antigen-binding fragments thereof, may be expressed by the cells following introduction of nucleic acid sequences encoding the variant antibodies, or variant antigen-binding fragments thereof. A nucleic acid sequence encoding a variant antibody, or variant antigen-binding fragment thereof, may be incorporated into an expression cassette or an expression vector for subsequent introduction in a cell. An expression cassette may generally include a construct of genetic material that contains coding sequences of the variant antibody, or variant antigen-binding fragment thereof, and enough regulatory information, e.g., promoter, to direct proper transcription and/or translation of the coding sequences in the cell. The expression cassette may further be inserted into a vector such as a plasmid, cosmid, virus, replicon, autonomously replicating polynucleotide molecule or phage. A vector may be understood to refer to a recombinant polynucleotide construct that can be used for the purpose of transformation, e.g., the introduction of heterologous DNA into a cell.

Suitable vectors for use in eukaryotic and prokaryotic cells, for introducing the nucleic acid sequence encoding the variant antibody, or variant antigen-binding fragment thereof, are known in the art and are commercially available, or readily prepared by a skilled artisan. See for example, Sambrook, J., & Russell, D. W. (2012). Molecular Cloning: A Laboratory Manual (4th ed.). Cold Spring Harbor, NY: Cold Spring Harbor Laboratory and Sambrook, J., & Russel, D. W. (2001). Molecular Cloning: A Laboratory Manual (3rd ed.). Cold Spring Harbor, NY: Cold Spring Harbor Laboratory (jointly referred to herein as “Sambrook”); Ausubel, F. M. (1987). Current Protocols in Molecular Biology. New York, NY: Wiley (including supplements through 2014); Bollag, D. M. et al. (1996). Protein Methods. New York, NY: Wiley-Liss; Huang, L. et al. (2005). Nonviral Vectors for Gene Therapy. San Diego: Academic Press; Kaplitt, M. G. et al. (1995). Viral Vectors: Gene Therapy and Neuroscience Applications. San Diego, CA: Academic Press; Lefkovits, I. (1997). The Immunology Methods Manual: The Comprehensive Sourcebook of Techniques. San Diego, CA: Academic Press; Doyle, A. et al. (1998). Cell and Tissue Culture: Laboratory Procedures in Biotechnology. New York, NY: Wiley; Mullis, K. B., Ferre, F. & Gibbs, R. (1994). PCR: The Polymerase Chain Reaction. Boston: Birkhauser Publisher; Greenfield, E. A. (2014). Antibodies: A Laboratory Manual (2nd ed.). New York, NY: Cold Spring Harbor Laboratory Press; Beaucage, S. L. et al. (2000). Current Protocols in Nucleic Acid Chemistry. New York, NY: Wiley, (including supplements through 2014); and Makrides, S. C. (2003). Gene Transfer and Expression in Mammalian Cells. Amsterdam, NL: Elsevier Sciences B.V., the disclosures of which are incorporated herein by reference).

Selection of an appropriate cell and expression cassette or vector for expressing the variant antibody, or variant antigen-binding fragment thereof, may be made by one of skill in the art. Guidance, if required, for making such a selection, can be found, e.g., in P. Jones, “Vectors: Cloning Applications”, John Wiley and Sons, New York, N.Y., 2009).

Nucleic acid sequences encoding the variant antibodies, or variant antigen-binding fragments thereof, can be introduced into cells for expression by any method known in the art including, e.g., viral infection, transfection, conjugation, protoplast fusion, lipofection, electroporation, nucleofection, calcium phosphate precipitation, polyethyleneimine (PEI)-mediated transfection, DEAE-dextran mediated transfection, liposome-mediated transfection, particle gun technology, calcium phosphate precipitation, direct micro-injection, or nanoparticle-mediated nucleic acid delivery. If the nucleic acid sequences encoding the variant antibodies, or variant antigen-binding fragments thereof, are introduced into mammalian cells, the nucleic acid sequences may be introduced by dextran-mediated transfection, calcium phosphate precipitation, polybrene-mediated transfection, protoplast fusion, electroporation, encapsulation of the nucleic acid molecule(s) in liposomes, lipid nanoparticle technology, biolistic injection, direct microinjection of the DNA into nuclei, or via viral vectors such as lentivirus or adeno-associated virus.

Incubating a Plurality of Cells with the Target Antigen in a Reaction Mixture

In some embodiments of the methods provided herein, the plurality of cells expressing the variant antibodies, or variant antigen-binding fragments thereof, may be incubated in a reaction mixture with the target antigen. In such embodiments, the target antigen may be coupled to a reporter oligonucleotide.

In addition to the reaction mixture including cells of the plurality of cells expressing variant antibodies, or variant antigen-binding fragments thereof, the reaction mixture may include a non-target antigen coupled to a second reporter oligonucleotide. The non-target antigen may be any antigen to which the variant antibodies or variant antigen-binding fragments thereof, would not be expected to bind. By way of example, if the provided nucleic acid sequence of the selected antibody, or selected antigen-binding fragment thereof, was derived from human B cells, then the non-target antigen may be any antigen for which a human would not be expected to develop an antibody response to or to have antibodies with a specificity for. Such a non-target antigen may be an antigen endogenous to and abundantly expressed in a human, e.g., human serum albumin.

The reporter oligonucleotide, coupled to the target antigen, may include a reporter barcode sequence that identifies the target antigen and a capture handle sequence. The second reporter oligonucleotide, coupled to the non-target antigen, may include a second reporter barcode that identifies the non-target antigen and the capture handle sequence.

In methods in which the plurality of cells is incubated in a reaction mixture with the target antigen, coupled to a reporter oligonucleotide, the reaction mixture may be partitioned into a plurality of partitions. The partitioning of the reaction mixture may also be referred to as the compartmentalization or depositing of the reaction mixture into discrete compartments or partitions, where each partition maintains separation of its own contents from the contents of other partitions. Examples of partitions include a droplet or well. Additional detail regarding partitions, and systems for partitions is provided in greater detail in the “Partitions, Partitioning, Reagents and Processing” section of the disclosure herein and below.

The partitioning of the reaction mixture provides a plurality of partitions. A partition of the plurality of partitions may include: (i) a cell bound to the target antigen, which is coupled to a reporter oligonucleotide; and (ii) a plurality of nucleic acid barcode molecules that comprise a partition-specific barcode sequence. A cell bound to the target antigen, which is coupled to a reporter oligonucleotide, may further include a detectable label. The detectable label may be coupled to the reporter oligonucleotide via a labelling of the target antigen or via a labeling of a nucleotide(s) of the reporter oligonucleotide. The detectable label may be fluorescent or it may be magnetic.

Nucleic acid barcode molecules of the plurality of nucleic acid barcode molecules in the partition may include a partition-specific barcode sequence. A partition-specific barcode sequence of the nucleic acid barcode molecule may identify the partition in which the nucleic acid barcode molecule is partitioned. Nucleic acid barcode molecules of the plurality of nucleic acid barcode molecules may further include a capture sequence. A capture sequence may be configured to couple to the capture handle sequence of a reporter oligonucleotide, e.g., by complementary base pairing. A capture sequence may also be configured to couple to a capture handle sequence of an mRNA or a DNA analyte of the cell, e.g., by complementary base pairing.

If the capture sequence is configured to couple to an mRNA or a DNA analyte, it may configured to couple to an mRNA analyte, and it may include a polyT sequence. If the capture sequence is configured to couple to a DNA analyte, the DNA analyte may be a cDNA analyte, and the capture sequence may be configured to couple to non-templated nucleotides appended to the cDNA when reverse transcribed from an mRNA analyte. The cDNA may be reverse transcribed from the mRNA using a primer having a polydT sequence. If the cDNA is reverse transcribed from the mRNA, whether by using a primer having a polydT or other sequence, the non-templated nucleotides are appended to the cDNA during reverse transcription. The non-templated nucleotides may be one, or at least one, or two, or at least two, or three, or at least three cytosines. If the non-templated nucleotides appended to the cDNA reverse transcribed from the mRNA analyte are the cytosines, then the capture sequence of the nucleic acid barcode molecules may be include one or more guanines that couple to the cytosines via complementary base pairing. Coupling of the non-templated nucleotide(s) appended to the cDNA to the capture sequence of the nucleic acid barcode molecules extends reverse transcription of the cDNA into the nucleic acid barcode molecules of the plurality of nucleic acid barcode molecules.

It will be understood that the partitioning of the reaction mixture may partition more than one cell of the plurality of cells into more than one of a plurality of partitions. The partitioning of the reaction mixture may partition a first cell of the plurality of cells into a first partition, it may further partition a second cell of the plurality of cells into a second partition. Moreover, it may additionally partition a third cell of the plurality of cells into a third partition, a fourth cell of the plurality of cells into a fourth partition, up to hundreds of cells that are partitioned into separate, individual, partitions. It should be understood that, in the partitioning, it is possible that not all partitions will include a cell. It should also be understood that, in the partitioning, not all partitions that include a cell will have the cell bound to the target antigen. Nonetheless, at least one cell of the population of cells partitioned into a partition will be bound to the target antigen.

In the methods in which the reaction mixture is partitioned, barcoded nucleic acid molecules, which may include a first and a second barcoded nucleic acid molecule, may be generated in the partition. The first barcoded nucleic acid molecule may include a sequence of the reporter oligonucleotide or a reverse complement thereof and the partition-specific barcode sequence or reverse complement thereof. The second barcoded nucleic acid molecule may include a nucleic acid sequence encoding the variant antibody, or variant antigen-binding fragment thereof, expressed by the cell or reverse complement thereof and the partition-specific barcode sequence or reverse complement thereof.

It will be understood that the first and/or the second barcoded nucleic acid molecules may further include a UMI. The UMI may be a sequence originating from a reporter oligonucleotide or a nucleic acid barcode molecule. It will be understood that any of the barcoded nucleic acid molecules may further include a functional sequence. Functional sequences are disclosed herein, e.g., and include flow cell attachment sequences or sequencing primer sequences.

Identifying the Variant Antibody or Variant Antigen-Binding Fragment Thereof

The variant antibody, or variant antigen-binding fragment thereof, may be identified as engineered to have the altered characteristic based on the generated first barcoded nucleic acid molecule. The variant antibody, or variant antigen-binding fragment thereof, may be identified for inclusion in the library based on the generated first barcoded nucleic acid molecule. Identification of the variant antibody, or variant antigen-binding fragment thereof, based on the generated first nucleic acid molecule may be by a determination of the quantity or number of counts of the first barcoded nucleic acid molecules generated in the partition. If the first barcoded nucleic acid molecule generated in the partition includes a UMI sequence, the quantity or number of counts of the UMI sequence may be used to identify the variant antibody, or variant antigen-binding fragment thereof, as having the altered characteristic or for inclusion in the library. The quantity or number of first barcoded nucleic acid molecules, or quantity/number of UMI counts, generated in partition may be used determine binding affinity of the variant antibody, or variant antigen-binding fragment thereof for the target antigen.

The quantity or number of counts of first barcoded nucleic acid molecules, or UMI sequences of first barcoded nucleic acid molecules, generated in a first partition including a cell expressing the variant antibody, or variant antigen-binding fragment thereof, may be compared to the quantity or number of counts of first barcoded nucleic acid molecules, or UMI sequences of first barcoded nucleic acid molecules, generated in a second partition including a cell expressing the selected antibody, or selected antigen-binding fragment thereof. A higher number/quantity of counts associated with the first barcoded nucleic acid molecules, or UMI sequences of first barcoded nucleic acid molecules, generated in the first partition relative to the second partition may identify the variant antibody or variant antigen-binding fragment thereof as having the altered characteristic or for inclusion in the library.

A higher number/quantity of counts associated with the first barcoded nucleic acid molecules, or UMI sequences of first barcoded nucleic acid molecules, in the first partition relative to the second partition may be 5% higher, 10% higher, 15% higher, 20% higher, 25% higher, 30% higher, 35% higher, 40% higher, 45% higher 50% higher, 55% higher, 60% higher, 65% higher, 70% higher, 75% higher, 80% higher, 85% higher, 90% higher, 95% higher, 100% higher, 150% higher, or 200% higher number/quantity of counts. A higher number/quantity of counts associated with the first barcoded nucleic acid molecules, or UMI sequences of first barcoded nucleic acid molecules, in the first partition relative to the second partition may be at least 5% higher, at least 10% higher, at least 15% higher, at least 20% higher, at least 25% higher, at least 30% higher, at least 35% higher, at least 40% higher, at least 45% higher at least 50% higher, at least 55% higher, at least 60% higher, at least 65% higher, at least 70% higher, at least 75% higher, at least 80% higher, at least 85% higher, at least 90% higher, at least 95% higher, at least 100% higher, at least 150% higher or at least 200% higher number/quantity of counts. In this instance, the altered characteristic identified for the variant antibody, or variant antigen-binding fragment thereof, or that identified the variant antibody, or variant antigen-binding fragment thereof, for inclusion in the library, may be increased affinity for the target antigen.

A lower number/quantity of counts associated with the first barcoded nucleic acid molecules, or UMI sequences of first barcoded nucleic acid molecules, in the first partition relative to the second partition may also identify the variant antibody or variant antigen-binding fragment thereof as having the altered characteristic or for inclusion in the library. The number/quantity of counts associated with the first barcoded nucleic acid molecules, or UMI sequences of first barcoded nucleic acid molecules, in the first partition relative to the second partition may be 1% lower, 2% lower, 3% lower, 4% lower, 5% lower, 10% lower, 15% lower or 20% lower. It may be at most 1% lower, at most 2% lower, at most 3% lower, at most 4% lower, at most 5% lower, at most 10% lower, at most 15% lower or at most 20% lower. In this instance, the altered characteristic identified for the variant antibody, or variant antigen-binding fragment thereof, may or may not (due to assay variability) be decreased affinity for the target antigen. A variant antibody (or variant antigen-fragment thereof) identified as a having an altered characteristic of decreased (or potentially decreased) affinity for the target antigen may be acceptable if the affinity of the variant antibody, or variant antigen-binding fragment thereof, for the target antigen remains in a tolerable range and the identified variant antibody, or variant antigen-binding fragment thereof, exhibits further altered, characteristics such as an altered association constant, an altered dissociation constant, or altered specificity for the target antigen, (such as a specificity alteration from multispecificity to more selective binding to the target antigen relative to the selected antibody, or selected antigen-binding fragment thereof). In another embodiment, the number/quantity of counts associated with the first barcoded nucleic acid molecules, or UMI sequences of first barcoded nucleic acid molecules, in the first partition relative to the second partition may be 100% lower. A variant antibody (or variant antigen-fragment thereof) identified as a having an altered characteristic of 100% decreased affinity for the target antigen may be useful if it is desirable for the variant antibody, or variant antigen-binding fragment thereof, if multispecific, to exhibit selective binding to a second, but not the, target antigen.

It will be understood that, to identify the variant antibody, or variant antigen-binding fragment thereof, as having the altered characteristic or for inclusion in a library, it is not necessary to determine the quantity or number of counts of first barcoded nucleic acid molecules, or UMI sequences of the first barcoded nucleic acid molecules, in a partition including a cell expressing the selected antibody, or selected antigen-binding fragment thereof. Rather, the first barcoded nucleic acid molecules generated in partitions including cells expressing variant antibodies, or variant antigen-binding fragments thereof, alone may be used for the identification. The identification may be made by determining that the generated first barcoded nucleic acid molecules, or UMI sequences of the first barcoded nucleic acid molecules, of partitions of cells that express the variant antibodies, or variant antigen-binding fragments there, are in a certain predetermined range. The predetermined range may be determined based on known characteristics of antibodies, generally, or the known characteristics of the selected antibody, or selected antigen-binding fragment thereof.

If the altered characteristic is determined to be affinity by number/quantity of counts associated with first barcoded nucleic acid molecules, or UMI sequences of first barcoded nucleic acid molecules, the binding affinity may be confirmed by other techniques that determine affinity of antigen-binding molecules for target proteins and/or their regions of interest including, for example, competition binning and competition enzyme-linked immunosorbent assay (ELISA), NMR or HDX-MS, Surface Plasmon Resonance (SPR), e.g. by using a Biacore™ system, or KinExA.

It will be understood that an altered characteristic of a variant antibody, or variant antigen-binding fragment thereof, may be, or may further include an alteration in an activity mediated by the variant relative to the selected antibody, or antigen-binding fragment thereof. An altered activity may be altered neutralization, if the selected antibody, or selected antigen-binding fragment thereof, is selected due to its binding a target antigen that is associated with a pathogen, e.g., a virus. An altered activity may be altered autoimmune activity, if the selected antibody, or selected antigen-binding fragment thereof, is selected due to its binding a target antigen that is associated with inflammation, e.g., cytokine or cytokine receptor. An altered activity may be altered anti-tumor activity, if the selected antibody or selected antigen-binding fragment thereof, is selected due to its binding a target antigen that is associated with a growth factor or growth factor receptor implicated in cancer, e.g., EGFR or IGFR.

Sequences encoding the variant antibody, or variant antigen-binding fragment thereof, may also be determined by the generated barcoded nucleic acid molecules. Sequences encoding the variant antibody, or variant antigen-binding fragment thereof, may be determined from the second barcoded nucleic acid sequence. The determined sequences of the variant antibody, or variant antigen-binding fragment thereof, from the second barcoded nucleic acid sequence may be sequences encoding one or more of a complementarity determining region (CDR), a framework (FWR), a variable heavy chain domain (VH), and/or a variable light chain domain (VL) of the antibody or antigen-binding fragment thereof.

Sequencing may be by performed by any of a variety of approaches, systems, or techniques, including next-generation sequencing (NGS) methods. Sequencing may be performed using nucleic acid amplification, polymerase chain reaction (PCR) (e.g., digital PCR and droplet digital PCR (ddPCR), quantitative PCR, real time PCR, multiplex PCR, PCR-based singleplex methods, emulsion PCR), and/or isothermal amplification. Non-limiting examples of nucleic acid sequencing methods include Maxam-Gilbert sequencing and chain-termination methods, de novo sequencing methods including shotgun sequencing and bridge PCR, next-generation methods including Polony sequencing, 454 pyrosequencing, Illumina sequencing, SOLiD™ sequencing, Ion Torrent semiconductor sequencing, HeliScope single molecule sequencing, nanopore sequencing (see, e.g., Oxford Nanopore Technologies), and SMRT® sequencing.

Further, sequence analysis of the nucleic acid molecules can be direct or indirect. Thus, the sequence analysis can be performed on a barcoded nucleic acid molecule or it can be a molecule which is derived therefrom (e.g., a complement thereof).

Other examples of methods for sequencing include, but are not limited to, DNA hybridization methods, restriction enzyme digestion methods, Sanger sequencing methods, ligation methods, and microarray methods. Additional examples of sequencing methods that can be used include targeted sequencing, single molecule real-time sequencing, exon sequencing, electron microscopy-based sequencing, panel sequencing, transistor-mediated sequencing, direct sequencing, random shotgun sequencing, Sanger dideoxy termination sequencing, whole-genome sequencing, sequencing by hybridization, pyrosequencing, capillary electrophoresis, gel electrophoresis, duplex sequencing, cycle sequencing, single-base extension sequencing, solid-phase sequencing, high-throughput sequencing, massively parallel signature sequencing, co-amplification at lower denaturation temperature-PCR (COLD-PCR), sequencing by reversible dye terminator, paired-end sequencing, near-term sequencing, exonuclease sequencing, sequencing by ligation, short-read sequencing, single-molecule sequencing, sequencing-by-synthesis, real-time sequencing, reverse-terminator sequencing, nanopore sequencing, Solexa Genome Analyzer sequencing, MS-PET sequencing, whole transcriptome sequencing, and any combinations thereof.

A more detailed understanding of steps of methods provided herein that include embodiments and elements related to partitions, partitioning, reporter oligonucleotides and generation of barcoded nucleic acid molecules are provided in the immediately following “Partitions, Partitioning, Reagents and Processing” below. An advantage of embodiments of the methods including elements related to paritions, partitioning, reporter oligonucleotides and generation of barcoded nucleic acid molecules is the rapid identification of variant antibodies, or variant antigen-binding fragments thereof having altered, e.g., improved, characteristics. When compared to more traditional lead antibody identification, optimization and screening approaches, these embodiments of the methods are able to reduce the time to optimize and identify antibodies, or antigen-binding fragments thereof, as clinical candidates down to a period of weeks to months. For example, such embodiments of the methods are able to reduce the time to optimize and identify antibodies, or antigen-binding fragments thereof, as clinical candidates down to a period of 2-6 weeks. Furthermore, in embodiments in which the provided nucleic acid sequence encoding the selected antibody, or selected antigen-binding fragment thereof, is derived from nucleic acids of a cell of a human donor, any variant of the selected antibody (or selected antigen-binding fragment thereof) identified by these particular embodiments of the methods will be derisked for immunogenicity, e.g., and will have more predictable pharmacokinetics, in a recipient when employed in a therapeutic setting

### Partitions, Partitioning, Reagents and Processing

Systems and Methods for Partitioning

In some aspects, the methods provided herein may include a step of partitioning, or may include a step of generating barcoded nucleic acid molecules, or may include additional processing step(s) as a result of having performed a step of partitioning and/or a step of generating barcoded nucleic acid molecules. This description sets forth examples, embodiments and characteristics of these steps, and reagents useful in the performing these steps.

In an aspect, the systems and methods described herein provide for the compartmentalization, depositing, or partitioning of one or more particles (e.g., biological particles, macromolecular constituents of biological particles, beads, reagents, etc.) into discrete compartments or partitions (referred to interchangeably herein as partitions), where each partition maintains separation of its own contents from the contents of other partitions.

In some embodiments disclosed herein, the partitioned biological particle is a cell, or a human cell, a cell expressing a variant antibody (or variant antigen-binding fragment thereof), or any cell which expresses an antibody, or antigen-binding fragment thereof. In particular embodiments, the cell expresses the antibody or antigen-binding fragment thereof on its surface. In other examples, the partitioned particle can be a labelled cell engineered to express antibodies or functional fragments thereof.

The term “partition,” as used herein, generally, refers to a space or volume that can be suitable to contain one or more cells, one or more species of features or compounds, or conduct one or more reactions. A partition can be a physical container, compartment, or vessel, such as a droplet, a flow cell, a reaction chamber, a reaction compartment, a tube, a well, or a microwell. In some embodiments, the compartments or partitions include partitions that are flowable within fluid streams. These partitions can include, for example, micro-vesicles that have an outer barrier surrounding an inner fluid center or core, or, in some cases, the partitions can include a porous matrix that is capable of entraining and/or retaining materials within its matrix. In some aspects, partitions comprise droplets of aqueous fluid within a non-aqueous continuous phase (e.g., oil phase). A variety of different vessels are described in, for example, U.S. Patent Application Publication No. 2014/0155295. Emulsion systems for creating stable droplets in non-aqueous or oil continuous phases are described in detail in, e.g., U.S. Patent Application Publication No. 2010/010511.

In some embodiments, a partition herein includes a space or volume that can be suitable to contain one or more species or conduct one or more reactions. A partition can be a physical compartment, such as a droplet or well. The partition can be an isolated space or volume from another space or volume. The droplet can be a first phase (e.g., aqueous phase) in a second phase (e.g., oil) immiscible with the first phase. The droplet can be a first phase in a second phase that does not phase separate from the first phase, such as, for example, a capsule or liposome in an aqueous phase. A partition can include one or more other (inner) partitions. In some cases, a partition can be a virtual compartment that can be defined and identified by an index (e.g., indexed libraries) across multiple and/or remote physical compartments. For example, a physical compartment can include a plurality of virtual compartments.

In some embodiments, the methods described herein provide for the compartmentalization, depositing or partitioning of individual cells from a reaction mixture containing cells, into discrete partitions, where each partition maintains separation of its own contents from the contents of other partitions. Identifiers including unique identifiers (e.g., UMI) and common or universal tags, e.g., barcodes, can be previously, subsequently or concurrently delivered to the partitions that hold the compartmentalized or partitioned cells, in order to allow for the later attribution of the characteristics of the individual cells to one or more particular compartments. Further, identifiers including unique identifiers and common or universal tags, e.g., barcodes, can be coupled to labelling agents and previously, subsequently or concurrently delivered to the partitions that hold the compartmentalized or partitioned cells, in order to allow for the later attribution of the characteristics of the individual cells to one or more particular compartments. Identifiers including unique identifiers and common or universal tags, e.g., barcodes, can be delivered, for example on an oligonucleotide, to a partition via any suitable mechanism, for example by coupling the barcoded oligonucleotides to a bead. In some embodiments, the barcoded oligonucleotides are reversibly (e.g., releasably) coupled to a bead. The bead suitable for the compositions and methods of the disclosure can have different surface chemistries and/or physical volumes. In some embodiments, the bead includes a polymer gel. In some embodiments, the polymer gel is a polyacrylamide. Additional non-limiting examples of suitable beads include microparticles, nanoparticles, beads, and microbeads. The partition can be a droplet in an emulsion. A partition can include one or more particles. A partition can include one or more types of particles. For example, a partition of the present disclosure can include one or more biological particles, e.g., a cell, or a human cell, a mammalian B cell, a cell expressing a variant antibody (or variant antigen-binding fragment thereof), and/or macromolecular constituents thereof. A partition can include one or more gel beads. A partition can include one or more cell beads. A partition can include a single gel bead, a single cell bead, or both a single cell bead and single gel bead. A partition can include one or more reagents. Alternatively, a partition can be unoccupied. For example, a partition cannot comprise a bead. Unique identifiers, such as barcodes, can be injected into the droplets previous to, subsequent to, or concurrently with droplet generation, such as via a bead, as described elsewhere herein. Microfluidic channel networks (e.g., on a chip) can be utilized to generate partitions as described herein. Alternative mechanisms can also be employed in the partitioning of individual biological particles, including porous membranes through which aqueous mixtures of cells are extruded into non-aqueous fluids.

The partitions can be flowable within fluid streams. The partitions can include, for example, micro-vesicles that have an outer barrier surrounding an inner fluid center or core. In some cases, the partitions can include a porous matrix that is capable of entraining and/or retaining materials (e.g., expressed antibodies or antigen-binding fragments thereof) within its matrix (e.g., via a capture agent configured to couple to both the matrix and the expressed antibody or antigen-binding fragment thereof). The partitions can be droplets of a first phase within a second phase, wherein the first and second phases are immiscible. For example, the partitions can be droplets of aqueous fluid within a non-aqueous continuous phase (e.g., oil phase). In another example, the partitions can be droplets of a non-aqueous fluid within an aqueous phase. In some examples, the partitions can be provided in a water-in-oil emulsion or oil-in-water emulsion. A variety of different vessels are described in, for example, U.S. Patent Application Publication No. 2014/0155295. Emulsion systems for creating stable droplets in non-aqueous or oil continuous phases are described in, for example, U.S. Patent Application Publication No. 2010/0105112.

In the case of droplets in an emulsion, allocating individual particles (e.g., labelled cells, or human cells, or a cell expressing a variant antibody (or variant antigen-binding fragment thereof)) to discrete partitions can, in one non-limiting example, be accomplished by introducing a flowing stream of particles in an aqueous fluid into a flowing stream of a non-aqueous fluid, such that droplets are generated at the junction of the two streams. Fluid properties (e.g., fluid flow rates, fluid viscosities, etc.), particle properties (e.g., volume fraction, particle size, particle concentration, etc.), microfluidic architectures (e.g., channel geometry, etc.), and other parameters can be adjusted to control the occupancy of the resulting partitions (e.g., number of biological particles per partition, number of beads per partition, etc.). For example, partition occupancy can be controlled by providing the aqueous stream at a certain concentration and/or flow rate of particles. To generate single biological particle partitions, the relative flow rates of the immiscible fluids can be selected such that, on average, the partitions can contain less than one biological particle per partition in order to ensure that those partitions that are occupied are primarily singly occupied. In some cases, partitions among a plurality of partitions can contain at most one biological particle (e.g., bead, DNA, cell, human cell, cell expressing a variant antibody or variant antigen-binding fragment thereof, or cellular material). In some embodiments, the various parameters (e.g., fluid properties, particle properties, microfluidic architectures, etc.) can be selected or adjusted such that a majority of partitions are occupied, for example, allowing for only a small percentage of unoccupied partitions. The flows and channel architectures can be controlled as to ensure a given number of singly occupied partitions, less than a certain level of unoccupied partitions and/or less than a certain level of multiply occupied partitions.

In some embodiments, the method further includes individually partitioning one or more single cells from a plurality of cells in a partition of a second plurality of partitions.

In some embodiments, at least one of the first and second plurality of partitions includes a microwell, a flow cell, a reaction chamber, a reaction compartment, or a droplet. In some embodiments, at least one of the first and second plurality of partitions includes individual droplets in emulsion. In some embodiments, the partitions of the first plurality and/or the second plurality of partition have the same reaction volume.

In the case of droplets in emulsion, allocating individual cells to discrete partitions can generally be accomplished by introducing a flowing stream of cells in an aqueous fluid into a flowing stream of a non-aqueous fluid, such that droplets are generated at the junction of the two streams. By providing the aqueous cell-containing stream at a certain concentration of cells, the occupancy of the resulting partitions (e.g., number of cells per partition) can be controlled. For example, where single cell partitions are desired, the relative flow rates of the fluids can be selected such that, on average, the partitions contain less than one cell per partition, in order to ensure that those partitions that are occupied, are primarily singly occupied. In some embodiments, the relative flow rates of the fluids can be selected such that a majority of partitions are occupied, e.g., allowing for only a small percentage of unoccupied partitions. In some embodiments, the flows and channel architectures are controlled as to ensure a desired number of singly occupied partitions, less than a certain level of unoccupied partitions and less than a certain level of multiply occupied partitions.

In some embodiments, the methods described herein can be performed such that a majority of occupied partitions include no more than one cell per occupied partition. In some embodiments, the partitioning process is performed such that fewer than 25%, fewer than 20%, fewer than 15%, fewer than 10%, fewer than 5%, fewer than 2%, or fewer than 1% the occupied partitions contain more than one cell. In some embodiments, fewer than 20% of the occupied partitions include more than one cell. In some embodiments, fewer than 10% of the occupied partitions include more than one cell per partition. In some embodiments, fewer than 5% of the occupied partitions include more than one cell per partition. In some embodiments, it is desirable to avoid the creation of excessive numbers of empty partitions. For example, from a cost perspective and/or efficiency perspective, it may be desirable to minimize the number of empty partitions. While this can be accomplished by providing sufficient numbers of cells into the partitioning zone, the Poissonian distribution can optionally be used to increase the number of partitions that include multiple cells. As such, in some embodiments described herein, the flow of one or more of the cells, or other fluids directed into the partitioning zone are performed such that no more than 50% of the generated partitions, no more than 25% of the generated partitions, or no more than 10% of the generated partitions are unoccupied. Further, in some aspects, these flows are controlled so as to present non-Poissonian distribution of single occupied partitions while providing lower levels of unoccupied partitions. Restated, in some aspects, the above noted ranges of unoccupied partitions can be achieved while still providing any of the single occupancy rates described above. For example, in some embodiments, the use of the systems and methods described herein creates resulting partitions that have multiple occupancy rates of less than 25%, less than 20%, less than 15%), less than 10%, and in some embodiments, less than 5%, while having unoccupied partitions of less than 50%), less than 40%, less than 30%, less than 20%, less than 10%, and in some embodiments, less than 5%.

Although described in terms of providing substantially singly occupied partitions, above, in some embodiments, the methods as described herein include providing multiply occupied partitions, e.g., containing two, three, four or more cells and/or beads comprising nucleic acid barcode molecules within a single partition.

In some embodiments, the reporter oligonucleotides contained within a partition are distinguishable from the reporter oligonucleotides contained within other partitions of the plurality of partitions.

In some embodiments, it may be desirable to incorporate multiple different barcode sequences within a given partition, either attached to a single or multiple beads within the partition. For example, in some cases, a mixed, but known barcode sequences set can provide greater assurance of identification in the subsequent processing, e.g., by providing a stronger address or attribution of the barcodes to a given partition, as a duplicate or independent confirmation of the output from a given partition.

Microfluidic Channel Structures

Microfluidic channel networks (e.g., on a chip) can be utilized to generate partitions as described herein. Alternative mechanisms can also be employed in the partitioning of individual biological particles, including porous membranes through which aqueous mixtures of cells are extruded into non-aqueous fluids.

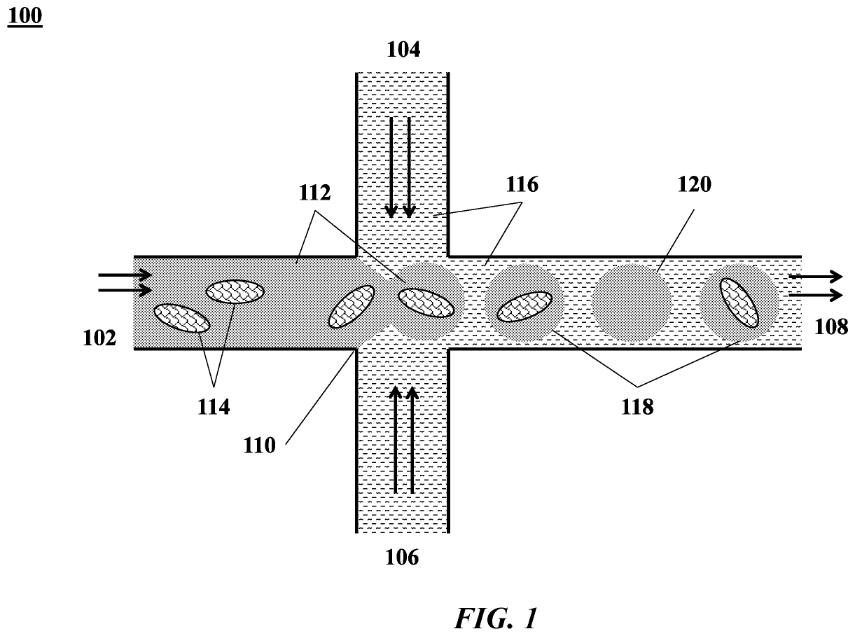

FIG. 1 shows an example of a microfluidic channel structure 100 for partitioning individual biological particles. The channel structure 100 can include channel segments 102, 104, 106 and 108 communicating at a channel junction 110. In operation, a first aqueous fluid 112 that includes suspended biological particles (e.g., cells, for example, human cells, or cells expressing a variant antibody (or variant antigen-binding fragment thereof)) 114 can be transported along channel segment 102 into junction 110, while a second fluid 116 that is immiscible with the aqueous fluid 112 is delivered to the junction 110 from each of channel segments 104 and 106 to create discrete droplets 118, 120 of the first aqueous fluid 112 flowing into channel segment 108, and flowing away from junction 110. The channel segment 108 can be fluidically coupled to an outlet reservoir where the discrete droplets can be stored and/or harvested. A discrete droplet generated can include an individual biological particle 114 (such as droplets 118). A discrete droplet generated can include more than one individual biological particle (e.g., labelled cells, for example, human cells, or cells expressing a variant antibody (or variant antigen-binding fragment thereof)) 114 (not shown in FIG. 1). A discrete droplet can contain no biological particle 114 (such as droplet 120). Each discrete partition can maintain separation of its own contents (e.g., individual biological particle 114) from the contents of other partitions.

The second fluid 116 can comprise an oil, such as a fluorinated oil, that includes a fluorosurfactant for stabilizing the resulting droplets, for example, inhibiting subsequent coalescence of the resulting droplets 118, 120. Examples of particularly useful partitioning fluids and fluorosurfactants are described, for example, in U.S. Patent Application Publication No. 2010/0105112.

As will be appreciated, the channel segments described herein can be coupled to any of a variety of different fluid sources or receiving components, including reservoirs, tubing, manifolds, or fluidic components of other systems. As will be appreciated, the microfluidic channel structure 100 can have other geometries. For example, a microfluidic channel structure can have more than one channel junction. For example, a microfluidic channel structure can have 2, 3, 4, or 5 channel segments each carrying particles (e.g., biological particles, cell beads, and/or gel beads) that meet at a channel junction. Fluid can be directed to flow along one or more channels or reservoirs via one or more fluid flow units. A fluid flow unit can comprise compressors (e.g., providing positive pressure), pumps (e.g., providing negative pressure), actuators, and the like to control flow of the fluid. Fluid can also or otherwise be controlled via applied pressure differentials, centrifugal force, electrokinetic pumping, vacuum, capillary or gravity flow, or the like.

The generated droplets can include two subsets of droplets: (1) occupied droplets 118, containing one or more biological particles 114, e.g. cells. Occupied droplets 118 can include singly occupied droplets (having one biological particle, such as one cell) and multiply occupied droplets (having more than one biological particle, such as cells). As described elsewhere herein, in some cases, the majority of occupied partitions can include no more than one biological particle, e.g., labelled cells, per occupied partition and some of the generated partitions can be unoccupied (of any biological particle, or labelled cells). In some cases, though, some of the occupied partitions can include more than one biological particle, e.g., labelled cells. In some cases, the partitioning process can be controlled such that fewer than about 25% of the occupied partitions contain more than one biological particle, and in many cases, fewer than about 20% of the occupied partitions have more than one biological particle, while in some cases, fewer than about 10% or even fewer than about 5% of the occupied partitions include more than one biological particle per partition.

In some cases, it can be desirable to minimize the creation of excessive numbers of empty partitions, such as to reduce costs and/or increase efficiency. While this minimization can be achieved by providing a sufficient number of biological particles (e.g., biological particles, such as labelled cells encapsulated in a partition, the Poissonian distribution can expectedly increase the number of partitions that include multiple biological particles. As such, where singly occupied partitions are to be obtained, at most about 95%, 90%, 85%, 80%, 75%, 70%, 65%, 60%, 55%, 50%, 45%, 40%, 35%, 30%, 25%, 20%, 15%, 10%, 5% or less of the generated partitions can be unoccupied.

In some cases, the flow of one or more of the biological particles, such as cells, (e.g., in channel segment 102), or other fluids directed into the partitioning junction (e.g., in channel segments 104, 106) can be controlled such that, in many cases, no more than about 50% of the generated partitions, no more than about 25% of the generated partitions, or no more than about 10% of the generated partitions are unoccupied. These flows can be controlled so as to present a non-Poissonian distribution of single-occupied partitions while providing lower levels of unoccupied partitions. The above noted ranges of unoccupied partitions can be achieved while still providing any of the single occupancy rates described above. For example, in many cases, the use of the systems and methods described herein can create resulting partitions that have multiple occupancy rates of less than about 25%, less than about 20%, less than about 15%, less than about 10%, and in many cases, less than about 5%, while having unoccupied partitions of less than about 50%, less than about 40%, less than about 30%, less than about 20%, less than about 10%, less than about 5%, or less.

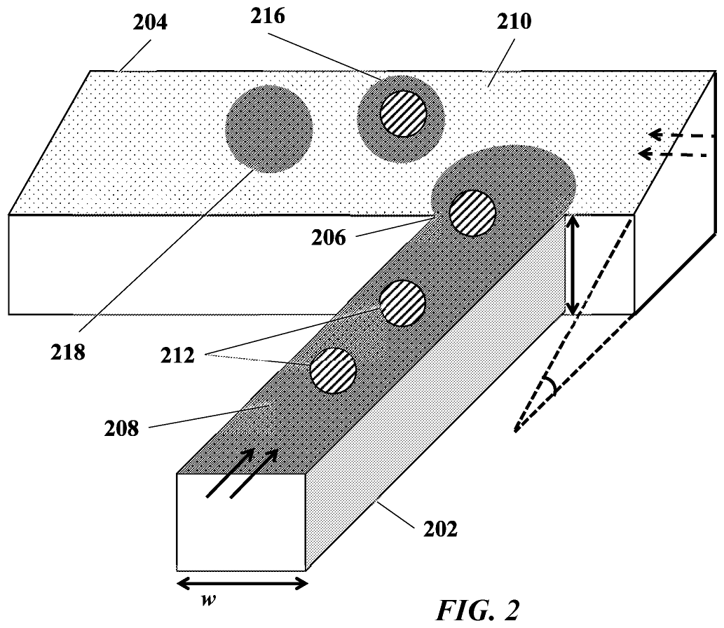

As will be appreciated, the above-described occupancy rates are also applicable to partitions that include both biological particles (e.g., cells) and additional reagents, including, but not limited to, beads (e.g., gel beads) carrying nucleic acid barcode molecules (e.g., barcoded oligonucleotides) (described in relation to FIGS. 1 and 2). The occupied partitions (e.g., at least about 10%, 20%, 30%, 40%, 50%, 60%, 70%, 80%, 90%, 95%, or 99% of the occupied partitions) can include both a bead comprising nucleic acid barcode molecules and a biological particle.

In another aspect, in addition to or as an alternative to droplet-based partitioning, biological particles (e.g., cells) may be encapsulated within a particulate material to form a “cell bead”.

The cell bead can include other reagents. Encapsulation of biological particles, e.g., labelled engineered cells, can be performed by a variety of processes. Such processes can combine an aqueous fluid containing the biological particles with a polymeric precursor material that can be capable of being formed into a gel or other solid or semi-solid matrix upon application of a particular stimulus to the polymer precursor. Such stimuli can include, for example, thermal stimuli (e.g., either heating or cooling), photo-stimuli (e.g., through photo-curing), chemical stimuli (e.g., through crosslinking, polymerization initiation of the precursor (e.g., through added initiators)), mechanical stimuli, or a combination thereof.

Encapsulation of biological particles, e.g., labelled cells, can be performed by a variety of methods. For example, air knife droplet or aerosol generators may be used to dispense droplets of precursor fluids into gelling solutions in order to form cell beads that include individual biological particles or small groups of biological particles. Likewise, membrane-based encapsulation systems may be used to generate cell beads comprising encapsulated biological particles as described herein. Microfluidic systems of the present disclosure, such as that shown in FIG. 1, may be readily used in encapsulating biological particles (e.g., cells) as described herein. Exemplary methods for encapsulating biological particles (e.g., cells) are also further described in U.S. Patent Application Pub. No. US 2015/0376609 and PCT/US2018/016019, which are hereby incorporated by reference in their entirety. In particular, and with reference to FIG. 1, the aqueous fluid 112 comprising (i) the biological particles 114 and (ii) the polymer precursor material (not shown) is flowed into channel junction 110, where it is partitioned into droplets 118, 120 through the flow of non-aqueous fluid 116. In the case of encapsulation methods, non-aqueous fluid 116 may also include an initiator (not shown) to cause polymerization and/or crosslinking of the polymer precursor to form the bead that includes the entrained biological particles. Examples of polymer precursor/initiator pairs include those described in U.S. Patent Application Publication No. 2014/0378345, which is entirely incorporated herein by reference in its entirety.

In some cases, encapsulated biological particles can be selectively releasable from the cell bead, such as through passage of time or upon application of a particular stimulus, that degrades the encapsulating material sufficiently to allow the biological particles (e.g., labelled cells), or its other contents to be released from the encapsulating material, such as into a partition (e.g., droplet). For example, in the case of the polyacrylamide polymer described above, degradation of the polymer can be accomplished through the introduction of an appropriate reducing agent, such as DTT or the like, to cleave disulfide bonds that cross-link the polymer matrix. See, for example, U.S. Patent Application Publication No. 2014/0378345 which is entirely incorporated herein by reference for all purposes.

The polymer or gel may be diffusively permeable to chemical or biochemical reagents. The polymer or gel may be diffusively impermeable to macromolecular constituents of the biological particle. In this manner, the polymer or gel may act to allow the biological particle to be subjected to chemical or biochemical operations while spatially confining the macromolecular constituents to a region of the droplet defined by the polymer or gel.

The polymer or gel may be functionalized to bind to targeted analytes, such as nucleic acids, proteins, carbohydrates, lipids or other analytes. In an embodiment, a first capture agent of a plurality of capture agents may be a polypeptide or aptamer that (i) couples or links to the backbone of the polymer, and (ii) binds a specific analyte (e.g., antibody or antigen-binding fragment thereof) secreted by the cell, e.g., B cell. By way of example, a first capture agent of a plurality of capture agents may be a polypeptide, e.g., antibody, or aptamer that couples/links to the backbone of the polymer and binds to a secreted antibody, e.g., at its Fc region, of a cell. It will be understood that, in some embodiments, the first capture agent of the plurality of capture agents may, rather than couple/link to the backbone of the polymer of the gel matrix, embed in/couple to the cell membrane. In these embodiments, the first capture agent, e.g., polypeptide or aptamer, may (i) embed in the membrane of the cell and/or bind to a cell surface protein and (ii) bind the specific analyte, e.g., antibody or antigen-binding fragment thereof, thereby tethering the secreted analyte, e.g., antibody, to the cell.

The polymer or gel may be polymerized or gelled via a passive mechanism. The polymer or gel may be stable in alkaline conditions or at elevated temperature. The polymer or gel may have mechanical properties similar to the mechanical properties of the bead. For instance, the polymer or gel may be of a similar size to the bead. The polymer or gel may have a mechanical strength (e.g. tensile strength) similar to that of the bead. The polymer or gel may be of a lower density than an oil. The polymer or gel may be of a density that is roughly similar to that of a buffer. The polymer or gel may have a tunable pore size. The pore size may be chosen to, for instance, retain denatured nucleic acids. The pore size may be chosen to maintain diffusive permeability to exogenous chemicals such as sodium hydroxide (NaOH) and/or endogenous chemicals such as inhibitors. The polymer or gel may be biocompatible. The polymer or gel may maintain or enhance cell viability. The polymer or gel may be biochemically compatible. The polymer or gel may be polymerized and/or depolymerized thermally, chemically, enzymatically, and/or optically.

The encapsulation of biological particles may constitute the partitioning of the biological particles into which other reagents are co-partitioned. Alternatively or in addition, encapsulated biological particles may be readily deposited into other partitions (e.g., droplets) as described above.

Microwells

As described herein, one or more processes can be performed in a partition, which can be a well. The well can be a well of a plurality of wells of a substrate, such as a microwell of a microwell array or plate, or the well can be a microwell or microchamber of a device (e.g., microfluidic device) comprising a substrate. The well can be a well of a well array or plate, or the well can be a well or chamber of a device (e.g., fluidic device). Accordingly, the wells or microwells can assume an “open” configuration, in which the wells or microwells are exposed to the environment (e.g., contain an open surface) and are accessible on one planar face of the substrate, or the wells or microwells can assume a “closed” or “sealed” configuration, in which the microwells are not accessible on a planar face of the substrate. In some instances, the wells or microwells can be configured to toggle between “open” and “closed” configurations. For instance, an “open” microwell or set of microwells can be “closed” or “sealed” using a membrane (e.g., semi-permeable membrane), an oil (e.g., fluorinated oil to cover an aqueous solution), or a lid, as described elsewhere herein. The wells or microwells can be initially provided in a “closed” or “sealed” configuration, wherein they are not accessible on a planar surface of the substrate without an external force. For instance, the “closed” or “sealed” configuration can include a substrate such as a sealing film or foil that is puncturable or pierceable by pipette tip(s). Suitable materials for the substrate include, without limitation, polyester, polypropylene, polyethylene, vinyl, and aluminum foil.

In some embodiments, the well can have a volume of less than 1 milliliter (mL). For example, the well can be configured to hold a volume of at most 1000 microliters (μL), at most 100 μL, at most 10 μL, at most 1 μL, at most 100 nanoliters (nL), at most 10 nL, at most 1 nL, at most 100 picoliters (μL), at most 10 (μL), or less. The well can be configured to hold a volume of about 1000 μL, about 100 μL, about 10 μL, about 1 μL, about 100 nL, about 10 nL, about 1 nL, about 100 μL, about 10 μL, etc. The well can be configured to hold a volume of at least 10 μL, at least 100 μL, at least 1 nL, at least 10 nL, at least 100 nL, at least 1 μL, at least 10 μL, at least 100 μL, at least 1000 μL, or more. The well can be configured to hold a volume in a range of volumes listed herein, for example, from about 5 nL to about 20 nL, from about 1 nL to about 100 nL, from about 500 μL to about 100 μL, etc. The well can be of a plurality of wells that have varying volumes and can be configured to hold a volume appropriate to accommodate any of the partition volumes described herein.

In some instances, a microwell array or plate includes a single variety of microwells. In some instances, a microwell array or plate includes a variety of microwells. For instance, the microwell array or plate can include one or more types of microwells within a single microwell array or plate. The types of microwells can have different dimensions (e.g., length, width, diameter, depth, cross-sectional area, etc.), shapes (e.g., circular, triangular, square, rectangular, pentagonal, hexagonal, heptagonal, octagonal, nonagonal, decagonal, etc.), aspect ratios, or other physical characteristics. The microwell array or plate can include any number of different types of microwells. For example, the microwell array or plate can include 1, 2, 3, 4, 5, 6, 7, 8, 9, 10, 20, 30, 40, 50, 60, 70, 80, 90, 100, 200, 300, 400, 500, 600, 700, 800, 900, 1000 or more different types of microwells. A well can have any dimension (e.g., length, width, diameter, depth, cross-sectional area, volume, etc.), shape (e.g., circular, triangular, square, rectangular, pentagonal, hexagonal, heptagonal, octagonal, nonagonal, decagonal, other polygonal, etc.), aspect ratios, or other physical characteristics described herein with respect to any well.

In certain instances, the microwell array or plate includes different types of microwells that are located adjacent to one another within the array or plate. For example, a microwell with one set of dimensions can be located adjacent to and in contact with another microwell with a different set of dimensions. Similarly, microwells of different geometries can be placed adjacent to or in contact with one another. The adjacent microwells can be configured to hold different articles; for example, one microwell can be used to contain a cell, cell bead, or other sample (e.g., cellular components, nucleic acid molecules, etc.) while the adjacent microwell can be used to contain a droplet, bead, or other reagent. In some cases, the adjacent microwells can be configured to merge the contents held within, e.g., upon application of a stimulus, or spontaneously, upon contact of the articles in each microwell.

As is described elsewhere herein, a plurality of partitions can be used in the systems, compositions, and methods described herein. For example, any suitable number of partitions (e.g., wells or droplets) can be generated or otherwise provided. For example, in the case when wells are used, at least about 1,000 wells, at least about 5,000 wells, at least about 10,000 wells, at least about 50,000 wells, at least about 100,000 wells, at least about 500,000 wells, at least about 1,000,000 wells, at least about 5,000,000 wells at least about 10,000,000 wells, at least about 50,000,000 wells, at least about 100,000,000 wells, at least about 500,000,000 wells, at least about 1,000,000,000 wells, or more wells can be generated or otherwise provided. Moreover, the plurality of wells can include both unoccupied wells (e.g., empty wells) and occupied wells.

A well can include any of the reagents described herein, or combinations thereof. These reagents can include, for example, barcode molecules, enzymes, adapters, and combinations thereof. The reagents can be physically separated from a sample (for example, a cell, cell bead, or cellular components, e.g., proteins, nucleic acid molecules, etc.) that is placed in the well. This physical separation can be accomplished by containing the reagents within, or coupling to, a bead that is placed within a well. The physical separation can also be accomplished by dispensing the reagents in the well and overlaying the reagents with a layer that is, for example, dissolvable, meltable, or permeable prior to introducing the polynucleotide sample into the well. This layer can be, for example, an oil, wax, membrane (e.g., semi-permeable membrane), or the like. The well can be sealed at any point, for example, after addition of the bead, after addition of the reagents, or after addition of either of these components. The sealing of the well can be useful for a variety of purposes, including preventing escape of beads or loaded reagents from the well, permitting select delivery of certain reagents (e.g., via the use of a semi-permeable membrane), for storage of the well prior to or following further processing, etc.

Once sealed, the well may be subjected to conditions for further processing of a cell (or cells) in the well. For instance, reagents in the well may allow further processing of the cell, e.g., cell lysis, as further described herein. Alternatively, the well (or wells such as those of a well-based array) comprising the cell (or cells) may be subjected to freeze-thaw cycling to process the cell (or cells), e.g., cell lysis. The well containing the cell may be subjected to freezing temperatures (e.g., 0° C., below 0° C., −5° C., −10° C., −15° C., −20° C., −25° C., −30° C., −35° C., −40° C., −45° C., −50° C., −55° C., −60° C., −65° C., −70° C., −80° C., or −85° C.). Freezing may be performed in a suitable manner, e.g., sub-zero freezer or a dry ice/ethanol bath. Following an initial freezing, the well (or wells) comprising the cell (or cells) may be subjected to freeze-thaw cycles to lyse the cell (or cells). In one embodiment, the initially frozen well (or wells) are thawed to a temperature above freezing (e.g., 4° C. or above, 8° C. or above, 12° C. or above, 16° C. or above, 20° C. or above, room temperature, or 25° C. or above). In another embodiment, the freezing is performed for less than 10 minutes (e.g., 5 minutes or 7 minutes) followed by thawing at room temperature for less than 10 minutes (e.g., 5 minutes or 7 minutes). This freeze-thaw cycle may be repeated a number of times, e.g., 2, 3, 4 or more times, to obtain lysis of the cell (or cells) in the well (or wells). In one embodiment, the freezing, thawing and/or freeze/thaw cycling is performed in the absence of a lysis buffer. Additional disclosure related to freeze-thaw cycling is provided in WO2019165181A1, which is incorporated herein by reference in its entirety.

A well can include free reagents and/or reagents encapsulated in, or otherwise coupled to or associated with, beads or droplets. In some embodiments, any of the reagents described in this disclosure can be encapsulated in, or otherwise coupled to, a droplet or bead, with any chemicals, particles, and elements suitable for sample processing reactions involving biomolecules, such as, but not limited to, nucleic acid molecules and proteins. For example, a bead or droplet used in a sample preparation reaction for DNA sequencing can include one or more of the following reagents: enzymes, restriction enzymes (e.g., multiple cutters), ligase, polymerase, fluorophores, oligonucleotide barcodes, adapters, buffers, nucleotides (e.g., dNTPs, ddNTPs) and the like.

Additional examples of reagents include, but are not limited to: buffers, acidic solution, basic solution, temperature-sensitive enzymes, pH-sensitive enzymes, light-sensitive enzymes, metals, metal ions, magnesium chloride, sodium chloride, manganese, aqueous buffer, mild buffer, ionic buffer, inhibitor, enzyme, protein, polynucleotide, antibodies, saccharides, lipid, oil, salt, ion, detergents, ionic detergents, non-ionic detergents, oligonucleotides, nucleotides, deoxyribonucleotide triphosphates (dNTPs), dideoxyribonucleotide triphosphates (ddNTPs), DNA, RNA, peptide polynucleotides, complementary DNA (cDNA), double stranded DNA (dsDNA), single stranded DNA (ssDNA), plasmid DNA, cosmid DNA, chromosomal DNA, genomic DNA, viral DNA, bacterial DNA, mtDNA (mitochondrial DNA), mRNA, rRNA, tRNA, nRNA, siRNA, snRNA, snoRNA, scaRNA, microRNA, dsRNA, ribozyme, riboswitch and viral RNA, polymerase, ligase, restriction enzymes, proteases, nucleases, protease inhibitors, nuclease inhibitors, chelating agents, reducing agents, oxidizing agents, fluorophores, probes, chromophores, dyes, organics, emulsifiers, surfactants, stabilizers, polymers, water, small molecules, pharmaceuticals, radioactive molecules, preservatives, antibiotics, aptamers, and pharmaceutical drug compounds. As described herein, one or more reagents in the well can be used to perform one or more reactions, including but not limited to: cell lysis, cell fixation, permeabilization, nucleic acid reactions, e.g., nucleic acid extension reactions, amplification, reverse transcription, transposase reactions (e.g., tagmentation), etc.

The wells disclosed herein can have been provided as a part of a kit. For example, such a kit can include instructions for use, a microwell array or device, and reagents (e.g., beads). The kit can include any useful reagents for performing the processes described herein, e.g., nucleic acid reactions, barcoding of nucleic acid molecules, sample processing (e.g., for cell lysis, fixation, and/or permeabilization).

In some cases, a well includes a bead or droplet that includes a set of reagents that has a similar attribute, for example, a set of enzymes, a set of minerals, a set of oligonucleotides, a mixture of different barcode molecules, a mixture of identical barcode molecules. In other cases, a bead or droplet includes a heterogeneous mixture of reagents. In some cases, the heterogeneous mixture of reagents can include all components necessary to perform a reaction. In some cases, such mixture can include all components necessary to perform a reaction, except for 1, 2, 3, 4, 5, or more components necessary to perform a reaction. In some cases, such additional components are contained within, or otherwise coupled to, a different droplet or bead, or within a solution within a partition (e.g., microwell) of the system.

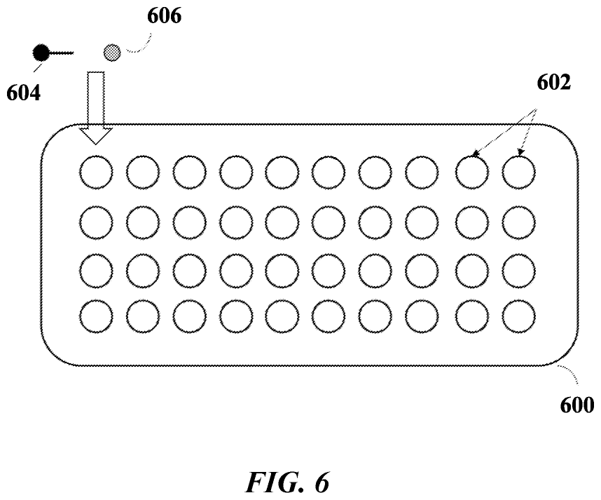

A non-limiting example of a microwell array in accordance with some embodiments of the disclosure is schematically presented in FIG. 6. In this example, the array can be contained within a substrate 600. The substrate 600 includes a plurality of wells 602. The wells 602 can be of any size or shape, and the spacing between the wells, the number of wells per substrate, as well as the density of the wells on the substrate 600 can be modified, depending on the particular application. In one such example application, a sample molecule 606, which can include a cell or cellular components (e.g., nucleic acid molecules) is co-partitioned with a bead 604, which can include a nucleic acid barcode molecule coupled thereto. The wells 602 can be loaded using gravity or other loading technique (e.g., centrifugation, liquid handler, acoustic loading, optoelectronic, etc.). In some instances, at least one of the wells 602 contains a single sample molecule 606 (e.g., cell) and a single bead 604.

Reagents can be loaded into a well either sequentially or concurrently. In some cases, reagents are introduced to the device either before or after a particular operation. In some cases, reagents (which can be provided, in certain instances, in droplets or beads) are introduced sequentially such that different reactions or operations occur at different steps. The reagents (or droplets, or beads) can also be loaded at operations interspersed with a reaction or operation step. For example, or droplets or beads including reagents for fragmenting polynucleotides (e.g., restriction enzymes) and/or other enzymes (e.g., transposases, ligases, polymerases, etc.) can be loaded into the well or plurality of wells, followed by loading of droplets or beads including reagents for attaching nucleic acid barcode molecules to a sample nucleic acid molecule. Reagents can be provided concurrently or sequentially with a sample, e.g., a cell or cellular components (e.g., organelles, proteins, nucleic acid molecules, carbohydrates, lipids, etc.). Accordingly, use of wells can be useful in performing multi-step operations or reactions.

As described elsewhere herein, the nucleic acid barcode molecules and other reagents can be contained within a bead or droplet. These beads or droplets can be loaded into a partition (e.g., a microwell) before, after, or concurrently with the loading of a cell, such that each cell is contacted with a different bead or droplet. This technique can be used to attach a unique nucleic acid barcode molecule to nucleic acid molecules obtained from each cell. Alternatively or in addition, the sample nucleic acid molecules can be attached to a support. For example, the partition (e.g., microwell) can include a bead which has coupled thereto a plurality of nucleic acid barcode molecules. The sample nucleic acid molecules, or derivatives thereof, can couple or attach to the nucleic acid barcode molecules attached on the support. The resulting barcoded nucleic acid molecules can then be removed from the partition, and in some instances, pooled and sequenced. In such cases, the nucleic acid barcode sequences can be used to trace the origin of the sample nucleic acid molecule. For example, polynucleotides with identical barcodes can be determined to originate from the same cell or partition, while polynucleotides with different barcodes can be determined to originate from different cells or partitions.

The samples or reagents can be loaded in the wells or microwells using a variety of approaches. For example, the samples (e.g., a cell, cell bead, or cellular component) or reagents (as described herein) can be loaded into the well or microwell using an external force, e.g., gravitational force, electrical force, magnetic force, or using mechanisms to drive the sample or reagents into the well, for example, via pressure-driven flow, centrifugation, optoelectronics, acoustic loading, electrokinetic pumping, vacuum, capillary flow, etc. In certain cases, a fluid handling system can be used to load the samples or reagents into the well. The loading of the samples or reagents can follow a Poissonian distribution or a non-Poissonian distribution, e.g., super Poisson or sub-Poisson. The geometry, spacing between wells, density, and size of the microwells can be modified to accommodate a useful sample or reagent distribution; for example, the size and spacing of the microwells can be adjusted such that the sample or reagents can be distributed in a super-Poissonian fashion.

In one non-limiting example, the microwell array or plate includes pairs of microwells, in which each pair of microwells is configured to hold a droplet (e.g., including a single cell) and a single bead (such as those described herein, which can, in some instances, also be encapsulated in a droplet). The droplet and the bead (or droplet containing the bead) can be loaded simultaneously or sequentially, and the droplet and the bead can be merged, e.g., upon contact of the droplet and the bead, or upon application of a stimulus (e.g., external force, agitation, heat, light, magnetic or electric force, etc.). In some cases, the loading of the droplet and the bead is super-Poissonian. In other examples of pairs of microwells, the wells are configured to hold two droplets including different reagents and/or samples, which are merged upon contact or upon application of a stimulus. In such instances, the droplet of one microwell of the pair can include reagents that can react with an agent in the droplet of the other microwell of the pair. For example, one droplet can include reagents that are configured to release the nucleic acid barcode molecules of a bead contained in another droplet, located in the adjacent microwell. Upon merging of the droplets, the nucleic acid barcode molecules can be released from the bead into the partition (e.g., the microwell or microwell pair that are in contact), and further processing can be performed (e.g., barcoding, nucleic acid reactions, etc.). In cases where intact or live cells are loaded in the microwells, one of the droplets can include lysis reagents for lysing the cell upon droplet merging.

In some embodiments, a droplet or bead can be partitioned into a well. The droplets can be selected or subjected to pre-processing prior to loading into a well. For instance, the droplets can include cells, and only certain droplets, such as those containing a single cell (or at least one cell), can be selected for use in loading of the wells. Such a pre-selection process can be useful in efficient loading of single cells, such as to obtain a non-Poissonian distribution, or to pre-filter cells for a selected characteristic prior to further partitioning in the wells. Additionally, the technique can be useful in obtaining or preventing cell doublet or multiplet formation prior to or during loading of the microwell.

In some embodiments, the wells can include nucleic acid barcode molecules attached thereto. The nucleic acid barcode molecules can be attached to a surface of the well (e.g., a wall of the well). The nucleic acid barcode molecules may be attached to a droplet or bead that has been partitioned into the well. The nucleic acid barcode molecule (e.g., a partition barcode sequence) of one well can differ from the nucleic acid barcode molecule of another well, which can permit identification of the contents contained with a single partition or well. In some embodiments, the nucleic acid barcode molecule can include a spatial barcode sequence that can identify a spatial coordinate of a well, such as within the well array or well plate. In some embodiments, the nucleic acid barcode molecule can include a unique molecular identifier for individual molecule identification. In some instances, the nucleic acid barcode molecules can be configured to attach to or capture a nucleic acid molecule within a sample or cell distributed in the well. For example, the nucleic acid barcode molecules can include a capture sequence that can be used to capture or hybridize to a nucleic acid molecule (e.g., RNA, DNA) within the sample. In some embodiments, the nucleic acid barcode molecules can be releasable from the microwell. In some instances, the nucleic acid barcode molecules may be releasable from the bead or droplet. For example, the nucleic acid barcode molecules can include a chemical crosslinker which can be cleaved upon application of a stimulus (e.g., photo-, magnetic, chemical, biological, stimulus). The released nucleic acid barcode molecules, which can be hybridized or configured to hybridize to a sample nucleic acid molecule, can be collected and pooled for further processing, which can include nucleic acid processing (e.g., amplification, extension, reverse transcription, etc.) and/or characterization (e.g., sequencing). In some instances nucleic acid barcode molecules attached to a bead or droplet in a well may be hybridized to sample nucleic acid molecules, and the bead with the sample nucleic acid molecules hybridized thereto may be collected and pooled for further processing, which can include nucleic acid processing (e.g., amplification, extension, reverse transcription, etc.) and/or characterization (e.g., sequencing). In such cases, the unique partition barcode sequences can be used to identify the cell or partition from which a nucleic acid molecule originated.

Characterization of samples within a well can be performed. Such characterization can include, in non-limiting examples, imaging of the sample (e.g., cell, cell bead, or cellular components) or derivatives thereof. Characterization techniques such as microscopy or imaging can be useful in measuring sample profiles in fixed spatial locations. For example, when cells are partitioned, optionally with beads, imaging of each microwell and the contents contained therein can provide useful information on cell doublet formation (e.g., frequency, spatial locations, etc.), cell-bead pair efficiency, cell viability, cell size, cell morphology, expression level of a biomarker (e.g., a surface marker, a fluorescently labeled molecule therein, etc.), cell or bead loading rate, number of cell-bead pairs, etc. In some instances, imaging can be used to characterize live cells in the wells, including, but not limited to: dynamic live-cell tracking, cell-cell interactions (when two or more cells are co-partitioned), cell proliferation, etc. Alternatively or in addition to, imaging can be used to characterize a quantity of amplification products in the well.

In operation, a well can be loaded with a sample and reagents, simultaneously or sequentially. When cells or cell beads are loaded, the well can be subjected to washing, e.g., to remove excess cells from the well, microwell array, or plate. Similarly, washing can be performed to remove excess beads or other reagents from the well, microwell array, or plate. In the instances where live cells are used, the cells can be lysed in the individual partitions to release the intracellular components or cellular analytes. Alternatively, the cells can be fixed or permeabilized in the individual partitions. The intracellular components or cellular analytes can couple to a support, e.g., on a surface of the microwell, on a solid support (e.g., bead), or they can be collected for further downstream processing. For example, after cell lysis, the intracellular components or cellular analytes can be transferred to individual droplets or other partitions for barcoding. Alternatively, or in addition, the intracellular components or cellular analytes (e.g., nucleic acid molecules) can couple to a bead including a nucleic acid barcode molecule; subsequently, the bead can be collected and further processed, e.g., subjected to nucleic acid reaction such as reverse transcription, amplification, or extension, and the nucleic acid molecules thereon can be further characterized, e.g., via sequencing. Alternatively, or in addition, the intracellular components or cellular analytes can be barcoded in the well (e.g., using a bead including nucleic acid barcode molecules that are releasable or on a surface of the microwell including nucleic acid barcode molecules). The barcoded nucleic acid molecules or analytes can be further processed in the well, or the barcoded nucleic acid molecules or analytes can be collected from the individual partitions and subjected to further processing outside the partition. Further processing can include nucleic acid processing (e.g., performing an amplification, extension) or characterization (e.g., fluorescence monitoring of amplified molecules, sequencing). At any suitable or useful step, the well (or microwell array or plate) can be sealed (e.g., using an oil, membrane, wax, etc.), which enables storage of the assay or selective introduction of additional reagents.

### Beads

In some embodiments of the disclosure, a partition can include one or more unique identifiers, such as barcodes (e.g., a plurality of nucleic acid barcode molecules which can be, for example, a plurality of partition barcode sequences). Barcodes can be previously, subsequently or concurrently delivered to the partitions that hold the compartmentalized or partitioned biological particle (e.g., labelled cells). For example, barcodes can be injected into droplets previous to, subsequent to, or concurrently with droplet generation. In some embodiments, the delivery of the barcodes to a particular partition allows for the later attribution of the characteristics of the individual biological particle (e.g., labelled cells) to the particular partition. Barcodes can be delivered, for example on a nucleic acid molecule (e.g., a barcoded oligonucleotide), to a partition via any suitable mechanism. In some embodiments, nucleic acid barcode molecules can be delivered to a partition via a bead. Beads are described in further detail below.

In some embodiments, nucleic acid barcode molecules can be initially associated with the bead and then released from the bead. In some embodiments, release of the nucleic acid barcode molecules can be passive (e.g., by diffusion out of the bead). In addition or alternatively, release from the bead can be upon application of a stimulus which allows the barcoded nucleic acid nucleic acid molecules to dissociate or to be released from the bead. Such stimulus can disrupt the bead, an interaction that couples the nucleic acid barcode molecules to or within the bead, or both. Such stimulus can include, for example, a thermal stimulus, photo-stimulus, chemical stimulus (e.g., change in pH or use of a reducing agent), a mechanical stimulus, a radiation stimulus; a biological stimulus (e.g., enzyme), or any combination thereof. Methods and systems for partitioning barcode carrying beads into droplets are provided in US. Patent Publication Nos. 2019/0367997 and 2019/0064173, and International Application Nos. PCT/US20/17785 and PCT/US20/020486.

Beneficially, a discrete droplet partitioning a biological particle and a barcode carrying bead can effectively allow the attribution of the barcode to macromolecular constituents of the biological particle within the partition. The contents of a partition can remain discrete from the contents of other partitions.

In operation, the barcoded oligonucleotides can be released (e.g., in a partition), as described elsewhere herein. Alternatively, the nucleic acid molecules bound to the bead (e.g., gel bead) can be used to hybridize and capture analytes (e.g., one or more types of analytes) on the solid phase of the bead.

In some examples, beads, biological particles (e.g., labelled cells, eukaryotic cells, or B cells) and droplets can flow along channels (e.g., the channels of a microfluidic device), in some cases at substantially regular flow profiles (e.g., at regular flow rates). Such regular flow profiles can permit a droplet to include a single bead and a single biological particle. Such regular flow profiles can permit the droplets to have an occupancy (e.g., droplets having beads and biological particles) greater than 5%, 10%, 20%, 30%, 40%, 50%, 60%, 70%, 80%, 90%, or 95%. Such regular flow profiles and devices that can be used to provide such regular flow profiles are provided in, for example, U.S. Patent Publication No. 2015/0292988.

A bead can be porous, non-porous, solid, semi-solid, semi-fluidic, fluidic, and/or a combination thereof. In some instances, a bead can be dissolvable, disruptable, and/or degradable. Degradable beads, as well as methods for degrading beads, are described in PCT/US2014/044398, which is hereby incorporated by reference in its entirety. In some cases, any combination of stimuli, e.g., stimuli described in PCT/US2014/044398 and US Patent Application Pub. No. 2015/0376609, hereby incorporated by reference in its entirety, may trigger degradation of a bead. For example, a change in pH may enable a chemical agent (e.g., DTT) to become an effective reducing agent.

In some cases, a bead cannot be degradable. In some cases, the bead can be a gel bead. A gel bead can be a hydrogel bead. A gel bead can be formed from molecular precursors, such as a polymeric or monomeric species. A semi-solid bead can be a liposomal bead. Solid beads can include metals including iron oxide, gold, and silver. In some cases, the bead can be a silica bead. In some cases, the bead can be rigid. In other cases, the bead can be flexible and/or compressible.

A bead can be of any suitable shape. Examples of bead shapes include, but are not limited to, spherical, non-spherical, oval, oblong, amorphous, circular, cylindrical, and variations thereof.

Beads can be of uniform size or heterogeneous size. In some cases, the diameter of a bead can be at least about 10 nanometers (nm), 100 nm, 500 nm, 1 micrometer (μm), 5 μm, 10 μm, 20 μm, 30 μm, 40 μm, 50 μm, 60 μm, 70 μm, 80 μm, 90 μm, 100 μm, 250 μm, 500 μm, 1 mm, or greater. In some cases, a bead can have a diameter of less than about 10 nm, 100 nm, 500 nm, 1 μm, 5 μm, 10 μm, 20 μm, 30 μm, 40 μm, 50 μm, 60 μm, 70 μm, 80 μm, 90 μm, 100 μm, 250 μm, 500 μm, 1 mm, or less. In some cases, a bead can have a diameter in the range of about 40-75 μm, 30-75 μm, 20-75 μm, 40-85 μm, 40-95 μm, 20-100 μm, 10-100 μm, 1-100 μm, 20-250 μm, or 20-500 μm.

In certain aspects, beads can be provided as a population or plurality of beads having a relatively monodisperse size distribution. Where it may be desirable to provide relatively consistent amounts of reagents within partitions, maintaining relatively consistent bead characteristics, such as size, can contribute to the overall consistency. In some embodiments, the beads described herein can have size distributions that have a coefficient of variation in their cross-sectional dimensions of less than 50%, less than 40%, less than 30%, less than 20%, and in some cases less than 15%, less than 10%, less than 5%, or less.

A bead can include natural and/or synthetic materials. For example, a bead can comprise a natural polymer, a synthetic polymer or both natural and synthetic polymers. See, e.g., PCT/US2014/044398, which is hereby incorporated by reference in its entirety. Beads may also be formed from materials other than polymers, including lipids, micelles, ceramics, glass-ceramics, material composites, metals, other inorganic materials, and others.

In some embodiments, the bead can include covalent or ionic bonds between polymeric precursors (e.g., monomers, oligomers, linear polymers), nucleic acid molecules (e.g., oligonucleotides), primers, and other entities. In some embodiments, the covalent bonds can be carbon-carbon bonds, thioether bonds, or carbon-heteroatom bonds.

In some embodiments, a bead can include an acrydite moiety, which in certain aspects can be used to attach one or more nucleic acid molecules (e.g., barcode sequence, nucleic acid barcode molecule, barcoded oligonucleotide, primer, or other oligonucleotide) to the bead.

For example, precursors (e.g., monomers, cross-linkers) that are polymerized to form a bead can include acrydite moieties, such that when a bead is generated, the bead also includes acrydite moieties. The acrydite moieties can be attached to a nucleic acid molecule (e.g., oligonucleotide, a nucleic acid barcode molecule described herein), which can include a priming sequence (e.g., a primer for amplifying target nucleic acids, random primer, primer sequence for messenger RNA) and/or one or more barcode sequences. The one or more barcode sequences can include sequences that are the same for all nucleic acid molecules coupled to a given bead and/or sequences that are different across all nucleic acid molecules coupled to the given bead. The nucleic acid molecule can be incorporated into the bead.

In some embodiments, the nucleic acid molecule can include a functional sequence, for example, for attachment to a sequencing flow cell, such as, for example, a P5 sequence for Illumina® sequencing. In some cases, the nucleic acid molecule or derivative thereof (e.g., oligonucleotide or polynucleotide generated from the nucleic acid molecule) can include another functional sequence, such as, for example, a P7 sequence for attachment to a sequencing flow cell for Illumina sequencing. In some cases, the nucleic acid molecule can include a barcode sequence. In some cases, the primer can further include a unique molecular identifier (UMI). In some cases, the primer can include an R1 primer sequence for Illumina sequencing. In some cases, the primer can include an R2 primer sequence for Illumina sequencing. Examples of such nucleic acid molecules (e.g., oligonucleotides, polynucleotides, etc.) and uses thereof, as can be used with compositions, devices, methods and systems of the present disclosure, are provided in U.S. Patent Pub. Nos. 2014/0378345 and 2015/0376609.

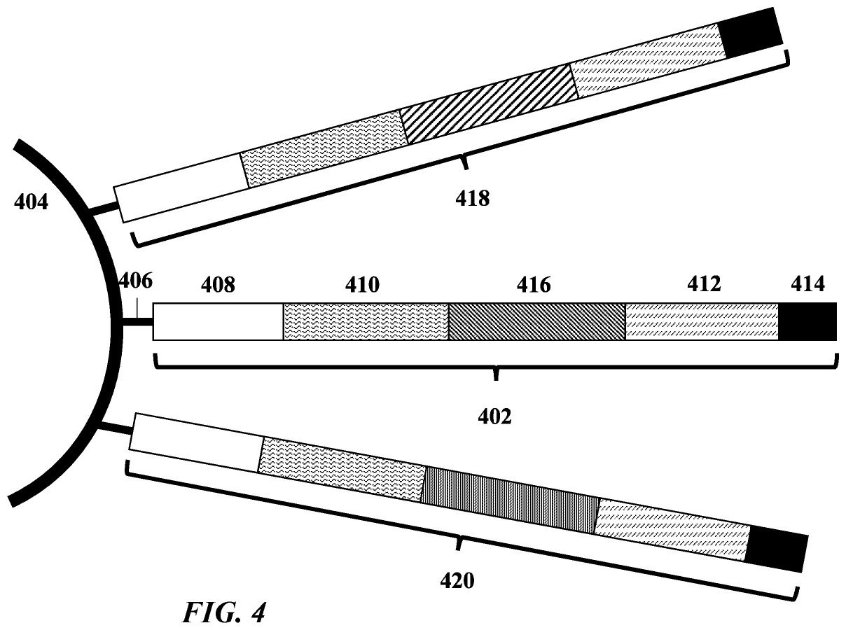

FIG. 4 illustrates an example of a barcode carrying bead. A nucleic acid molecule 402, such as an oligonucleotide, can be coupled to a bead 404 by a releasable linkage 406, such as, for example, a disulfide linker. The same bead 404 can be coupled (e.g., via releasable linkage) to one or more other nucleic acid molecules 418, 420. The nucleic acid molecule 402 can be or include a barcode. As noted elsewhere herein, the structure of the barcode can include a number of sequence elements. The nucleic acid molecule 402 can include a functional sequence 408 that can be used in subsequent processing. For example, the functional sequence 408 can include one or more of a sequencer specific flow cell attachment sequence (e.g., a P5 sequence for Illumina® sequencing systems) and a sequencing primer sequence (e.g., a R1 primer for Illumina® sequencing systems). The nucleic acid molecule 402 can include a barcode sequence 410 for use in barcoding the sample (e.g., DNA, RNA, protein, etc.). In some cases, the barcode sequence 410 can be bead-specific such that the barcode sequence 410 is common to all nucleic acid molecules (e.g., including nucleic acid molecule 402) coupled to the same bead 404. Alternatively or in addition, the barcode sequence 410 can be partition-specific such that the barcode sequence 410 is common to all nucleic acid molecules coupled to one or more beads that are partitioned into the same partition. The nucleic acid molecule 402 can include a specific priming sequence 412, such as an mRNA specific priming sequence (e.g., poly-T sequence), a targeted priming sequence, and/or a random priming sequence. The nucleic acid molecule 402 can include an anchoring sequence 414 to ensure that the specific priming sequence 412 hybridizes at the sequence end (e.g., of the mRNA). For example, the anchoring sequence 414 can include a random short sequence of nucleotides, such as a 1-mer, 2-mer, 3-mer or longer sequence, which can ensure that a poly-T segment is more likely to hybridize at the sequence end of the poly-A tail of the mRNA.

The nucleic acid molecule 402 can include a unique molecular identifying sequence 416 (e.g., unique molecular identifier (UMI)). In some cases, the unique molecular identifying sequence 416 can include from about 5 to about 8 nucleotides. Alternatively, the unique molecular identifying sequence 416 can compress less than about 5 or more than about 8 nucleotides. The unique molecular identifying sequence 416 can be a unique sequence that varies across individual nucleic acid molecules (e.g., 402, 418, 420, etc.) coupled to a single bead (e.g., bead 404). In some cases, the unique molecular identifying sequence 416 can be a random sequence (e.g., such as a random N-mer sequence). For example, the UMI can provide a unique identifier of the starting mRNA molecule that was captured, in order to allow quantitation of the number of original expressed RNA. As will be appreciated, although FIG. 4 shows three nucleic acid molecules 402, 418, 420 coupled to the surface of the bead 404, an individual bead can be coupled to any number of individual nucleic acid molecules, for example, from one to tens to hundreds of thousands or even millions of individual nucleic acid molecules. The respective barcodes for the individual nucleic acid molecules can include both common sequence segments or relatively common sequence segments (e.g., 408, 410, 412, etc.) and variable or unique sequence segments (e.g., 416) between different individual nucleic acid molecules coupled to the same bead.

In operation, a biological particle (e.g., cell, DNA, RNA, etc.) can be co-partitioned along with a barcode bearing bead 404. The nucleic acid barcode molecules 402, 418, 420 can be released from the bead 404 in the partition. By way of example, in the context of analyzing sample RNA, the poly-T segment (e.g., 412) of one of the released nucleic acid molecules (e.g., 402) can hybridize to the poly-A tail of a mRNA molecule. Reverse transcription can result in a cDNA transcript of the mRNA, but which transcript includes each of the sequence segments 408, 410, 416 of the nucleic acid molecule 402. Because the nucleic acid molecule 402 includes an anchoring sequence 414, it will more likely hybridize to and prime reverse transcription at the sequence end of the poly-A tail of the mRNA. Within any given partition, all of the cDNA transcripts of the individual mRNA molecules can include a common barcode sequence segment 410. However, the transcripts made from the different mRNA molecules within a given partition can vary at the unique molecular identifying sequence 412 segment (e.g., UMI segment).

Beneficially, even following any subsequent amplification of the contents of a given partition, the number of different UMIs can be indicative of the quantity of mRNA originating from a given partition, and thus from the biological particle (e.g., cell). As noted above, the transcripts can be amplified, cleaned up and sequenced to identify the sequence of the cDNA transcript of the mRNA, as well as to sequence the barcode segment and the UMI segment. While a poly-T primer sequence is described, other targeted or random priming sequences can also be used in priming the reverse transcription reaction. Likewise, although described as releasing the barcoded oligonucleotides into the partition, in some cases, the nucleic acid molecules bound to the bead (e.g., gel bead) can be used to hybridize and capture the mRNA on the solid phase of the bead, for example, in order to facilitate the separation of the RNA from other cell contents. In such cases, further processing can be performed, in the partitions or outside the partitions (e.g., in bulk). For instance, the RNA molecules on the beads can be subjected to reverse transcription or other nucleic acid processing, additional adapter sequences can be added to the barcoded nucleic acid molecules, or other nucleic acid reactions (e.g., amplification, nucleic acid extension) can be performed. The beads or products thereof (e.g., barcoded nucleic acid molecules) can be collected from the partitions, and/or pooled together and subsequently subjected to clean up and further characterization (e.g., sequencing).

The operations described herein can be performed at any useful or suitable step. For instance, the beads including nucleic acid barcode molecules can be introduced into a partition (e.g., well or droplet) prior to, during, or following introduction of a sample into the partition. The nucleic acid molecules of a sample can be subjected to barcoding, which can occur on the bead (in cases where the nucleic acid molecules remain coupled to the bead) or following release of the nucleic acid barcode molecules into the partition. In cases where analytes from the sample are captured by the nucleic acid barcode molecules in a partition (e.g., by hybridization), captured analytes from various partitions may be collected, pooled, and subjected to further processing (e.g., reverse transcription, adapter attachment, amplification, clean up, sequencing). For example, in cases wherein the nucleic acid molecules from the sample remain attached to the bead, the beads from various partitions can be collected, pooled, and subjected to further processing (e.g., reverse transcription, adapter attachment, amplification, clean up, and/or sequencing). In other instances, the processing can occur in the partition. For example, conditions sufficient for barcoding, adapter attachment, reverse transcription, or other nucleic acid processing operations can be provided in the partition and performed prior to clean up and sequencing.

In some instances, a bead can include a capture sequence or binding sequence configured to bind to a corresponding capture sequence or binding sequence. In some instances, a bead can include a plurality of different capture sequences or binding sequences configured to bind to different respective corresponding capture sequences or binding sequences. For example, a bead can include a first subset of one or more capture sequences each configured to bind to a first corresponding capture sequence, a second subset of one or more capture sequences each configured to bind to a second corresponding capture sequence, a third subset of one or more capture sequences each configured to bind to a third corresponding capture sequence, and etc. A bead can include any number of different capture sequences. In some instances, a bead can include at least 2, 3, 4, 5, 6, 7, 8, 9, 10 or more different capture sequences or binding sequences configured to bind to different respective capture sequences or binding sequences, respectively. Alternatively or in addition, a bead can include at most about 10, 9, 8, 7, 6, 5, 4, 3, or 2 different capture sequences or binding sequences configured to bind to different respective capture sequences or binding sequences. In some instances, the different capture sequences or binding sequences can be configured to facilitate analysis of a same type of analyte. In some instances, the different capture sequences or binding sequences can be configured to facilitate analysis of different types of analytes (with the same bead). The capture sequence can be designed to attach to a corresponding capture sequence. Beneficially, such corresponding capture sequence can be introduced to, or otherwise induced in, a biological particle (e.g., cell, cell bead, etc.) for performing different assays in various formats (e.g., barcoded antibodies including the corresponding capture sequence, barcoded MHC dextramers including the corresponding capture sequence, barcoded guide RNA molecules including the corresponding capture sequence, etc.), such that the corresponding capture sequence can later interact with the capture sequence associated with the bead. In some instances, a capture sequence coupled to a bead (or other support) can be configured to attach to a linker molecule, such as a splint molecule, wherein the linker molecule is configured to couple the bead (or other support) to other molecules through the linker molecule, such as to one or more analytes or one or more other linker molecules.

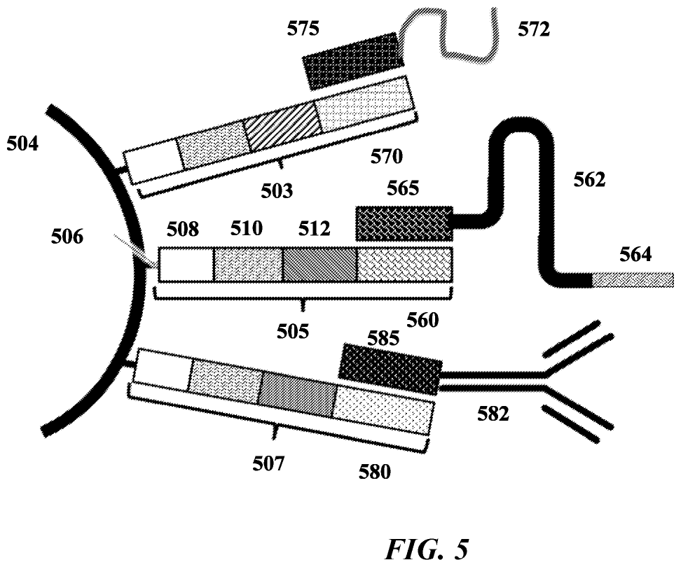

FIG. 5 illustrates a non-limiting example of a barcode carrying bead in accordance with some embodiments of the disclosure. A nucleic acid molecule 505, such as an oligonucleotide, can be coupled to a bead 504 by a releasable linkage 506, such as, for example, a disulfide linker. The nucleic acid molecule 505 can include a first capture sequence 560. The same bead 504 can be coupled, e.g., via releasable linkage, to one or more other nucleic acid molecules 503, 507 including other capture sequences. The nucleic acid molecule 505 can be or include a barcode. As described elsewhere herein, the structure of the barcode can include a number of sequence elements, such as a functional sequence 508 (e.g., flow cell attachment sequence, sequencing primer sequence, etc.), a barcode sequence 510 (e.g., bead-specific sequence common to bead, partition-specific sequence common to partition, etc.), and a unique molecular identifier 512 (e.g., unique sequence within different molecules attached to the bead), or partial sequences thereof. The capture sequence 560 can be configured to attach to a corresponding capture sequence 565 (e.g., capture handle). In some instances, the corresponding capture sequence 565 can be coupled to another molecule that can be an analyte or an intermediary carrier. For example, as illustrated in FIG. 5, the corresponding capture sequence 565 is coupled to a guide RNA molecule 562 including a target sequence 564, wherein the target sequence 564 is configured to attach to the analyte. Another oligonucleotide molecule 507 attached to the bead 504 includes a second capture sequence 580 which is configured to attach to a second corresponding capture sequence (e.g., capture handle) 585. As illustrated in FIG. 5, the second corresponding capture sequence 585 is coupled to an antibody 582. In some cases, the antibody 582 can have binding specificity to an analyte (e.g., surface protein). Alternatively, the antibody 582 cannot have binding specificity. Another oligonucleotide molecule 503 attached to the bead 504 includes a third capture sequence 570 which is configured to attach to a second corresponding capture sequence 575. As illustrated in FIG. 5, the third corresponding capture sequence (e.g., capture handle) 575 is coupled to a molecule 572. The molecule 572 may or may not be configured to target an analyte. The other oligonucleotide molecules 503, 507 can include the other sequences (e.g., functional sequence, barcode sequence, UMI, etc.) described with respect to oligonucleotide molecule 505. While a single oligonucleotide molecule including each capture sequence is illustrated in FIG. 5, it will be appreciated that, for each capture sequence, the bead can include a set of one or more oligonucleotide molecules each including the capture sequence. For example, the bead can include any number of sets of one or more different capture sequences. Alternatively or in addition, the bead 504 can include other capture sequences. Alternatively or in addition, the bead 504 can include fewer types of capture sequences (e.g., two capture sequences). Alternatively or in addition, the bead 504 can include oligonucleotide molecule(s) including a priming sequence, such as a specific priming sequence such as an mRNA specific priming sequence (e.g., poly-T sequence), a targeted priming sequence, and/or a random priming sequence, for example, to facilitate an assay for gene expression.

The generation of a barcoded sequence is described herein.

In some embodiments, precursors including a functional group that is reactive or capable of being activated such that it becomes reactive can be polymerized with other precursors to generate gel beads including the activated or activatable functional group. The functional group can then be used to attach additional species (e.g., disulfide linkers, primers, other oligonucleotides, etc.) to the gel beads. For example, some precursors including a carboxylic acid (COOH) group can co-polymerize with other precursors to form a gel bead that also includes a COOH functional group. In some cases, acrylic acid (a species including free COOH groups), acrylamide, and bis(acryloyl)cystamine can be co-polymerized together to generate a gel bead including free COOH groups. The COOH groups of the gel bead can be activated (e.g., via 1-Ethyl-3-(3-dimethylaminopropyl)carbodiimide (EDC) and N-Hydroxysuccinimide (NHS) or 4-(4,6-Dimethoxy-1,3,5-triazin-2-yl)-4-methylmorpholinium chloride (DMTMM)) such that they are reactive (e.g., reactive to amine functional groups where EDC/NHS or DMTMM are used for activation). The activated COOH groups can then react with an appropriate species (e.g., a species including an amine functional group where the carboxylic acid groups are activated to be reactive with an amine functional group) including a moiety to be linked to the bead.

Beads including disulfide linkages in their polymeric network can be functionalized with additional species via reduction of some of the disulfide linkages to free thiols. The disulfide linkages can be reduced via, for example, the action of a reducing agent (e.g., DTT, TCEP, etc.) to generate free thiol groups, without dissolution of the bead. Free thiols of the beads can then react with free thiols of a species or a species including another disulfide bond (e.g., via thiol-disulfide exchange) such that the species can be linked to the beads (e.g., via a generated disulfide bond). In some cases, free thiols of the beads can react with any other suitable group. For example, free thiols of the beads can react with species including an acrydite moiety. The free thiol groups of the beads can react with the acrydite via Michael addition chemistry, such that the species including the acrydite is linked to the bead. In some cases, uncontrolled reactions can be prevented by inclusion of a thiol capping agent such as N-ethylmaleimide or iodoacetate.

Activation of disulfide linkages within a bead can be controlled such that only a small number of disulfide linkages are activated. Methods of controlling activation of disulfide linkages within a bead are described in PCT/US2014/044398, which is hereby incorporated by reference in its entirety.

A bead injected or otherwise introduced into a partition can include releasably, cleavably, or reversibly attached barcodes (e.g., partition barcode sequences). A bead injected or otherwise introduced into a partition can include activatable barcodes. A bead injected or otherwise introduced into a partition can be degradable, disruptable, or dissolvable beads.

Barcodes can be releasably, cleavably or reversibly attached to the beads such that barcodes can be released or be releasable through cleavage of a linkage between the barcode molecule and the bead, or released through degradation of the underlying bead itself, allowing the barcodes to be accessed or be accessible by other reagents, or both. In non-limiting examples, cleavage can be achieved through reduction of di-sulfide bonds, use of restriction enzymes, photo-activated cleavage, or cleavage via other types of stimuli (e.g., chemical, thermal, pH, enzymatic, etc.) and/or reactions, such as described elsewhere herein. Releasable barcodes can sometimes be referred to as being activatable, in that they are available for reaction once released. Thus, for example, an activatable barcode can be activated by releasing the barcode from a bead (or other suitable type of partition described herein). Other activatable configurations are also envisioned in the context of the described methods and systems.

In addition to, or as an alternative to the cleavable linkages between the beads and the associated molecules, such as barcode containing nucleic acid molecules (e.g., barcoded oligonucleotides), the beads can be degradable, disruptable, or dissolvable spontaneously or upon exposure to one or more stimuli (e.g., temperature changes, pH changes, exposure to particular chemical species or phase, exposure to light, reducing agent, etc.). In some cases, a bead can be dissolvable, such that material components of the beads are solubilized when exposed to a particular chemical species or an environmental change, such as a change temperature or a change in pH. In some cases, a gel bead can be degraded or dissolved at elevated temperature and/or in basic conditions. In some cases, a bead can be thermally degradable such that when the bead is exposed to an appropriate change in temperature (e.g., heat), the bead degrades. Degradation or dissolution of a bead bound to a species (e.g., a nucleic acid molecule, e.g., barcoded oligonucleotide) can result in release of the species from the bead.

As will be appreciated from the above disclosure, the degradation of a bead can refer to the disassociation of a bound (e.g., capture agent configured to couple to a secreted antibody or antigen-binding fragment thereof) or entrained species (e.g., labelled cells) from a bead, both with and without structurally degrading the physical bead itself. For example, the degradation of the bead can involve cleavage of a cleavable linkage via one or more species and/or methods described elsewhere herein. In another example, entrained species can be released from beads through osmotic pressure differences due to, for example, changing chemical environments. See, e.g., PCT/US2014/044398, which is hereby incorporated by reference in its entirety.

A degradable bead can be introduced into a partition, such as a droplet of an emulsion or a well, such that the bead degrades within the partition and any associated species (e.g., oligonucleotides) are released within the droplet when the appropriate stimulus is applied. The free species (e.g., oligonucleotides, nucleic acid molecules) can interact with other reagents contained in the partition. See, e.g., PCT/US2014/044398, which is hereby incorporated by reference in its entirety.

Any suitable number of molecular tag molecules (e.g., primer, barcoded oligonucleotide) can be associated with a bead such that, upon release from the bead, the molecular tag molecules (e.g., primer, e.g., barcoded oligonucleotide) are present in the partition at a pre-defined concentration. Such pre-defined concentration can be selected to facilitate certain reactions for generating a sequencing library, e.g., amplification, within the partition. In some cases, the pre-defined concentration of the primer can be limited by the process of producing nucleic acid molecule (e.g., oligonucleotide) bearing beads.

In some cases, beads can be non-covalently loaded with one or more reagents. The beads can be non-covalently loaded by, for instance, subjecting the beads to conditions sufficient to swell the beads, allowing sufficient time for the reagents to diffuse into the interiors of the beads, and subjecting the beads to conditions sufficient to de-swell the beads. The swelling of the beads can be accomplished, for instance, by placing the beads in a thermodynamically favorable solvent, subjecting the beads to a higher or lower temperature, subjecting the beads to a higher or lower ion concentration, and/or subjecting the beads to an electric field. The swelling of the beads can be accomplished by various swelling methods. The de-swelling of the beads can be accomplished, for instance, by transferring the beads in a thermodynamically unfavorable solvent, subjecting the beads to lower or high temperatures, subjecting the beads to a lower or higher ion concentration, and/or removing an electric field. The de-swelling of the beads can be accomplished by various de-swelling methods. Transferring the beads can cause pores in the bead to shrink. The shrinking can then hinder reagents within the beads from diffusing out of the interiors of the beads. The hindrance can be due to steric interactions between the reagents and the interiors of the beads. The transfer can be accomplished microfluidically. For instance, the transfer can be achieved by moving the beads from one co-flowing solvent stream to a different co-flowing solvent stream. The swellability and/or pore size of the beads can be adjusted by changing the polymer composition of the bead.

In some cases, an acrydite moiety linked to a precursor, another species linked to a precursor, or a precursor itself can include a labile bond, such as chemically, thermally, or photo-sensitive bond e.g., disulfide bond, UV sensitive bond, or the like. Once acrydite moieties or other moieties including a labile bond are incorporated into a bead, the bead can also include the labile bond. The labile bond can be, for example, useful in reversibly linking (e.g., covalently linking) species (e.g., barcodes, primers, etc.) to a bead. In some cases, a thermally labile bond can include a nucleic acid hybridization based attachment, e.g., where an oligonucleotide is hybridized to a complementary sequence that is attached to the bead, such that thermal melting of the hybrid releases the oligonucleotide, e.g., a barcode containing sequence, from the bead.

The addition of multiple types of labile bonds to a gel bead can result in the generation of a bead capable of responding to varied stimuli. See, e.g., PCT/US2014/044398, which is hereby incorporated by reference in its entirety.

In addition to thermally cleavable bonds, disulfide bonds and UV sensitive bonds, other non-limiting examples of labile bonds that can be coupled to a precursor or bead include an ester linkage (e.g., cleavable with an acid, a base, or hydroxylamine), a vicinal diol linkage (e.g., cleavable via sodium periodate), a Diels-Alder linkage (e.g., cleavable via heat), a sulfone linkage (e.g., cleavable via a base), a silyl ether linkage (e.g., cleavable via an acid), a glycosidic linkage (e.g., cleavable via an amylase), a peptide linkage (e.g., cleavable via a protease), or a phosphodiester linkage (e.g., cleavable via a nuclease (e.g., DNAase)). A bond can be cleavable via other nucleic acid molecule targeting enzymes, such as restriction enzymes (e.g., restriction endonucleases), as described further below.

Species can be encapsulated in beads (e.g., capture agent) during bead generation (e.g., during polymerization of precursors). Such species may or may not participate in polymerization. Such species can be entered into polymerization reaction mixtures such that generated beads include the species upon bead formation. In some cases, such species can be added to the gel beads after formation. Such species can include, for example, nucleic acid molecules (e.g., oligonucleotides), reagents for a nucleic acid amplification reaction (e.g., primers, polymerases, dNTPs, co-factors (e.g., ionic co-factors, buffers) including those described herein, reagents for enzymatic reactions (e.g., enzymes, co-factors, substrates, buffers), reagents for nucleic acid modification reactions such as polymerization, ligation, or digestion, and/or reagents for template preparation (e.g., tagmentation) for one or more sequencing platforms (e.g., Nextera® for Illumina®). Such species can include one or more enzymes described herein, including without limitation, polymerase, reverse transcriptase, restriction enzymes (e.g., endonuclease), transposase, ligase, proteinase K, DNAse, etc. Such species can include one or more reagents described elsewhere herein (e.g., lysis agents, inhibitors, inactivating agents, chelating agents, stimulus). Trapping of such species can be controlled by the polymer network density generated during polymerization of precursors, control of ionic charge within the gel bead (e.g., via ionic species linked to polymerized species), or by the release of other species. Encapsulated species can be released from a bead upon bead degradation and/or by application of a stimulus capable of releasing the species from the bead. Alternatively or in addition, species can be partitioned in a partition (e.g., droplet) during or subsequent to partition formation. Such species can include, without limitation, the abovementioned species that can also be encapsulated in a bead.

A degradable bead can include one or more species with a labile bond such that, when the bead/species is exposed to the appropriate stimuli, the bond is broken and the bead degrades. The labile bond can be a chemical bond (e.g., covalent bond, ionic bond) or can be another type of physical interaction (e.g., van der Waals interactions, dipole-dipole interactions, etc.). In some cases, a crosslinker used to generate a bead can include a labile bond. Upon exposure to the appropriate conditions, the labile bond can be broken and the bead degraded. For example, upon exposure of a polyacrylamide gel bead including cystamine crosslinkers to a reducing agent, the disulfide bonds of the cystamine can be broken and the bead degraded.

A degradable bead can be useful in more quickly releasing an attached species (e.g., a nucleic acid molecule, a barcode sequence, a primer, etc.) from the bead when the appropriate stimulus is applied to the bead as compared to a bead that does not degrade. For example, for a species bound to an inner surface of a porous bead or in the case of an encapsulated species, the species can have greater mobility and accessibility to other species in solution upon degradation of the bead. In some cases, a species can also be attached to a degradable bead via a degradable linker (e.g., disulfide linker). The degradable linker can respond to the same stimuli as the degradable bead or the two degradable species can respond to different stimuli. For example, a barcode sequence can be attached, via a disulfide bond, to a polyacrylamide bead including cystamine. Upon exposure of the barcoded-bead to a reducing agent, the bead degrades and the barcode sequence is released upon breakage of both the disulfide linkage between the barcode sequence and the bead and the disulfide linkages of the cystamine in the bead.

As will be appreciated from the above disclosure, while referred to as degradation of a bead, in many instances as noted above, that degradation can refer to the disassociation of a bound or entrained species from a bead, both with and without structurally degrading the physical bead itself. For example, entrained species can be released from beads through osmotic pressure differences due to, for example, changing chemical environments. By way of example, alteration of bead pore sizes due to osmotic pressure differences can generally occur without structural degradation of the bead itself. In some cases, an increase in pore size due to osmotic swelling of a bead can permit the release of entrained species within the bead. In other cases, osmotic shrinking of a bead can cause a bead to better retain an entrained species due to pore size contraction.

Where degradable beads are provided, it can be beneficial to avoid exposing such beads to the stimulus or stimuli that cause such degradation prior to a given time, in order to, for example, avoid premature bead degradation and issues that arise from such degradation, including for example poor flow characteristics and aggregation. By way of example, where beads include reducible cross-linking groups, such as disulfide groups, it will be desirable to avoid contacting such beads with reducing agents, e.g., DTT or other disulfide cleaving reagents. In such cases, treatment to the beads described herein will, in some cases be provided free of reducing agents, such as DTT. Because reducing agents are often provided in commercial enzyme preparations, it can be desirable to provide reducing agent free (or DTT free) enzyme preparations in treating the beads described herein. Examples of such enzymes include, e.g., polymerase enzyme preparations, reverse transcriptase enzyme preparations, ligase enzyme preparations, as well as many other enzyme preparations that can be used to treat the beads described herein. The terms “reducing agent free” or “DTT free” preparations can refer to a preparation having less than about 1/10th, less than about 1/50th, or even less than about 1/100th of the lower ranges for such materials used in degrading the beads. For example, for DTT, the reducing agent free preparation can have less than about 0.01 millimolar (mM), 0.005 mM, 0.001 mM DTT, 0.0005 mM DTT, or even less than about 0.0001 mM DTT. In many cases, the amount of DTT can be undetectable.

Beads can also be induced to release their contents upon the application of a thermal stimulus. See, e.g., PCT/US2014/044398, which is hereby incorporated by reference in its entirety.

Any suitable agent can degrade beads. See, e.g., PCT/US2014/044398, which is hereby incorporated by reference in its entirety.

Any suitable number of molecular tag molecules (e.g., primer, barcoded oligonucleotide) can be associated with a bead such that, upon release from the bead, the molecular tag molecules (e.g., primer, e.g., barcoded oligonucleotide) are present in the partition at a pre-defined concentration. Such pre-defined concentration can be selected to facilitate certain reactions for generating a sequencing library, e.g., amplification, within the partition. In some cases, the pre-defined concentration of the primer can be limited by the process of producing oligonucleotide bearing beads.

Although FIG. 1 and FIG. 2 have been described in terms of providing substantially singly occupied partitions, above, in certain cases, it may be desirable to provide multiply occupied partitions, e.g., containing two, three, four or more cells and/or beads including nucleic acid barcode molecules (e.g., oligonucleotides) within a single partition (e.g., multi-omics method described elsewhere, herein). Accordingly, as noted above, the flow characteristics of the biological particle and/or bead containing fluids and partitioning fluids can be controlled to provide for such multiply occupied partitions. In particular, the flow parameters can be controlled to provide a given occupancy rate at greater than about 50% of the partitions, greater than about 75%, and in some cases greater than about 80%, 90%, 95%, or higher.

In some cases, additional beads can be used to deliver additional reagents to a partition. In such cases, it can be advantageous to introduce different beads into a common channel or droplet generation junction, from different bead sources (e.g., containing different associated reagents) through different channel inlets into such common channel or droplet generation junction (e.g., junction 110). In such cases, the flow and frequency of the different beads into the channel or junction can be controlled to provide for a certain ratio of beads from each source, while ensuring a given pairing or combination of such beads into a partition with a given number of biological particles (e.g., one biological particle and one bead per partition).

The partitions described herein can include small volumes, for example, less than about 10 microliters (L), 5 μL, 1 μL, 900 picoliters (μL), 800 μL, 700 μL, 600 μL, 500 μL, 400 μL, 300 μL, 200 μL, 100 μL, 50 μL, 20 μL, 10 μL, 1 μL, 500 nanoliters (nL), 100 nL, 50 nL, or less.

For example, in the case of droplet based partitions, the droplets can have overall volumes that are less than about 1000 μL, 900 μL, 800 μL, 700 μL, 600 μL, 500 μL, 400 μL, 300 μL, 200 μL, 100 μL, 50 μL, 20 μL, 10 μL, 1 μL, or less. Where co-partitioned with beads, it will be appreciated that the sample fluid volume, e.g., including co-partitioned biological particles and/or beads, within the partitions can be less than about 90% of the above described volumes, less than about 80%, less than about 70%, less than about 60%, less than about 50%, less than about 40%, less than about 30%, less than about 20%, or less than about 10% of the above described volumes.

As is described elsewhere herein, partitioning species can generate a population or plurality of partitions. In such cases, any suitable number of partitions can be generated or otherwise provided. For example, at least about 1,000 partitions, at least about 5,000 partitions, at least about 10,000 partitions, at least about 50,000 partitions, at least about 100,000 partitions, at least about 500,000 partitions, at least about 1,000,000 partitions, at least about 5,000,000 partitions at least about 10,000,000 partitions, at least about 50,000,000 partitions, at least about 100,000,000 partitions, at least about 500,000,000 partitions, at least about 1,000,000,000 partitions, or more partitions can be generated or otherwise provided. Moreover, the plurality of partitions can include both unoccupied partitions (e.g., empty partitions) and occupied partitions.

Reagents

In accordance with certain aspects, biological particles can be partitioned along with lysis reagents in order to release the contents of the biological particles within the partition. See, e.g., U.S. Pat. Pub. 2018/0216162 (now U.S. Pat. No. 10,428,326), U.S. Pat. Pub. 2019/0100632 (now U.S. Pat. No. 10,590,244), and U.S. Pat. Pub. 2019/0233878. Biological particles (e.g., cells, cell beads, cell nuclei, organelles, and the like) can be partitioned together with nucleic acid barcode molecules and the nucleic acid molecules of or derived from the biological particle (e.g., mRNA, cDNA, gDNA, etc.,) can be barcoded as described elsewhere herein. In some embodiments, biological particles are co-partitioned with barcode carrying beads (e.g., gel beads) and the nucleic acid molecules of or derived from the biological particle are barcoded as described elsewhere herein. In such cases, the lysis agents can be contacted with the biological particle suspension concurrently with, or immediately prior to, the introduction of the biological particles into the partitioning junction/droplet generation zone (e.g., junction 110), such as through an additional channel or channels upstream of the channel junction. In accordance with other aspects, additionally or alternatively, biological particles can be partitioned along with other reagents, as will be described further below.

Beneficially, when lysis reagents and biological particles are co-partitioned, the lysis reagents can facilitate the release of the contents of the biological particles within the partition. The contents released in a partition can remain discrete from the contents of other partitions.

As will be appreciated, the channel segments described herein can be coupled to any of a variety of different fluid sources or receiving components, including reservoirs, tubing, manifolds, or fluidic components of other systems. As will be appreciated, the microfluidic channel structures can have other geometries and/or configurations. For example, a microfluidic channel structure can have more than two channel junctions. For example, a microfluidic channel structure can have 2, 3, 4, 5 channel segments or more each carrying the same or different types of beads, reagents, and/or biological particles that meet at a channel junction. Fluid flow in each channel segment can be controlled to control the partitioning of the different elements into droplets. Fluid can be directed flow along one or more channels or reservoirs via one or more fluid flow units. A fluid flow unit can include compressors (e.g., providing positive pressure), pumps (e.g., providing negative pressure), actuators, and the like to control flow of the fluid. Fluid can also or otherwise be controlled via applied pressure differentials, centrifugal force, electrokinetic pumping, vacuum, capillary or gravity flow, or the like.

Examples of lysis agents include bioactive reagents, such as lysis enzymes that are used for lysis of different cell types, e.g., gram positive or negative bacteria, plants, yeast, mammalian, etc., such as lysozymes, achromopeptidase, lysostaphin, labiase, kitalase, lyticase, and a variety of other lysis enzymes available from, e.g., Sigma-Aldrich, Inc. (St Louis, MO), as well as other commercially available lysis enzymes. Other lysis agents can additionally or alternatively be co-partitioned with the biological particles to cause the release of the biological particle's contents into the partitions. For example, in some cases, surfactant-based lysis solutions can be used to lyse cells (e.g., labelled cells), although these can be less desirable for emulsion based systems where the surfactants can interfere with stable emulsions. In some cases, lysis solutions can include non-ionic surfactants such as, for example, Triton X-100 and Tween 20. In some cases, lysis solutions can include ionic surfactants such as, for example, sarcosyl and sodium dodecyl sulfate (SDS). Electroporation, thermal, acoustic or mechanical cellular disruption can also be used in certain cases, e.g., non-emulsion based partitioning such as encapsulation of biological particles that can be in addition to or in place of droplet partitioning, where any pore size of the encapsulate is sufficiently small to retain nucleic acid fragments of a given size, following cellular disruption.

Alternatively or in addition to the lysis agents co-partitioned with the biological particles (e.g., labelled cells) described above, other reagents can also be co-partitioned with the biological particles, including, for example, DNase and RNase inactivating agents or inhibitors, such as proteinase K, chelating agents, such as EDTA, and other reagents employed in removing or otherwise reducing negative activity or impact of different cell lysate components on subsequent processing of nucleic acids. In addition, in the case of encapsulated biological particles (e.g., cell beads comprising labelled cells), the biological particles can be exposed to an appropriate stimulus to release the biological particles or their contents from a co-partitioned cell bead. For example, in some cases, a chemical stimulus can be co-partitioned along with an encapsulated biological particle to allow for the degradation of the encapsulating material and release of the cell or its contents into the larger partition. In some cases, this stimulus can be the same as the stimulus described elsewhere herein for release of nucleic acid molecules (e.g., oligonucleotides) from their respective bead. In alternative aspects, this can be a different and non-overlapping stimulus, in order to allow an encapsulated biological particle to be released into a partition at a different time from the release of nucleic acid molecules into the same partition.

Additional reagents can also be co-partitioned with the biological particles (e.g., labelled cells), such as endonucleases to fragment a biological particle's DNA, DNA polymerase enzymes and dNTPs used to amplify the biological particle's nucleic acid fragments and to attach the barcode molecular tags to the amplified fragments. Other enzymes can be co-partitioned, including without limitation, polymerase, transposase, ligase, proteinase K, DNAse, etc. Additional reagents can also include reverse transcriptase enzymes, including enzymes with terminal transferase activity, primers and oligonucleotides, and switch oligonucleotides (also referred to herein as “switch oligos” or “template switching oligonucleotides”) which can be used for template switching. In some cases, template switching can be used to increase the length of a cDNA. In some cases, template switching can be used to append a predefined nucleic acid sequence to the cDNA. Template switching is further described in PCT/US2017/068320, which is hereby incorporated by reference in its entirety.

Template switching oligonucleotides may comprise a hybridization region and a template region. Template switching oligonucleotides are further described in PCT/US2017/068320, which is hereby incorporated by reference in its entirety. Once the contents of the cells (e.g., cells) are released into their respective partitions, the macromolecular components (e.g., macromolecular constituents of biological particles, such as RNA, DNA, proteins, or secreted antibodies or antigen-binding fragments thereof) contained therein can be further processed within the partitions. In accordance with the methods and systems described herein, the macromolecular component contents of individual biological particles (e.g., cells) can be provided with unique identifiers such that, upon characterization of those macromolecular components they can be attributed as having been derived from the same biological particle or particles. The ability to attribute characteristics to individual biological particles or groups of biological particles is provided by the assignment of unique identifiers specifically to an individual biological particle or groups of biological particles. Unique identifiers, e.g., in the form of nucleic acid barcodes can be assigned or associated with individual biological particles or populations of biological particles, in order to tag or label the biological particle's macromolecular components (and as a result, its characteristics) with the unique identifiers. These unique identifiers can then be used to attribute the biological particle's components and characteristics to an individual biological particle or group of biological particles.

In some aspects, this is performed by co-partitioning the individual biological particle (e.g., cells) or groups of biological particles (e.g., cells) with the unique identifiers, such as described above (with reference to FIGS. 1 and 2). In some aspects, the unique identifiers are provided in the form of nucleic acid molecules (e.g., oligonucleotides) that include nucleic acid barcode sequences that can be attached to or otherwise associated with the nucleic acid contents of individual biological particle, or to other components of the biological particle, and particularly to fragments of those nucleic acids. The nucleic acid molecules are partitioned such that as between nucleic acid molecules in a given partition, the nucleic acid barcode sequences contained therein are the same, but as between different partitions, the nucleic acid molecule can, and do have differing barcode sequences, or at least represent a large number of different barcode sequences across all of the partitions in a given analysis. In some aspects, only one nucleic acid barcode sequence can be associated with a given partition, although in some cases, two or more different barcode sequences can be present.

The nucleic acid barcode sequences can include from about 6 to about 20 or more nucleotides within the sequence of the nucleic acid molecules (e.g., oligonucleotides). The nucleic acid barcode sequences can include from about 6 to about 20, 30, 40, 50, 60, 70, 80, 90, 100 or more nucleotides. In some cases, the length of a barcode sequence can be about 6, 7, 8, 9, 10, 11, 12, 13, 14, 15, 16, 17, 18, 19, 20 nucleotides or longer. In some cases, the length of a barcode sequence can be at least about 6, 7, 8, 9, 10, 11, 12, 13, 14, 15, 16, 17, 18, 19, 20 nucleotides or longer. In some cases, the length of a barcode sequence can be at most about 6, 7, 8, 9, 10, 11, 12, 13, 14, 15, 16, 17, 18, 19, 20 nucleotides or shorter. These nucleotides can be completely contiguous, i.e., in a single stretch of adjacent nucleotides, or they can be separated into two or more separate subsequences that are separated by 1 or more nucleotides. In some cases, separated barcode subsequences can be from about 4 to about 16 nucleotides in length. In some cases, the barcode subsequence can be about 4, 5, 6, 7, 8, 9, 10, 11, 12, 13, 14, 15, 16 nucleotides or longer. In some cases, the barcode subsequence can be at least about 4, 5, 6, 7, 8, 9, 10, 11, 12, 13, 14, 15, 16 nucleotides or longer. In some cases, the barcode subsequence can be at most about 4, 5, 6, 7, 8, 9, 10, 11, 12, 13, 14, 15, 16 nucleotides or shorter.

The co-partitioned nucleic acid molecules can also include other functional sequences useful in the processing of the nucleic acids from the co-partitioned biological particles (e.g., labelled cells). These sequences include, e.g., targeted or random/universal amplification primer sequences for amplifying the genomic DNA from the individual biological particles within the partitions while attaching the associated barcode sequences, sequencing primers or primer recognition sites, hybridization or probing sequences, e.g., for identification of presence of the sequences or for pulling down barcoded nucleic acids, or any of a number of other potential functional sequences. Other mechanisms of co-partitioning oligonucleotides can also be employed, including, e.g., coalescence of two or more droplets, where one droplet contains oligonucleotides, or microdispensing of oligonucleotides into partitions, e.g., droplets within microfluidic systems.

In an example, beads are provided that each include large numbers of the above described nucleic acid barcode molecules (e.g., barcoded oligonucleotides) releasably attached to the beads, where all of the nucleic acid molecules attached to a particular bead will include the same nucleic acid barcode sequence, but where a large number of diverse barcode sequences are represented across the population of beads used. In some embodiments, hydrogel beads, e.g., including polyacrylamide polymer matrices, are used as a solid support and delivery vehicle for the nucleic acid molecules into the partitions, as they are capable of carrying large numbers of nucleic acid molecules, and can be configured to release those nucleic acid molecules upon exposure to a particular stimulus, as described elsewhere herein. In some cases, the population of beads provides a diverse barcode sequence library that includes at least about 1,000 different barcode sequences, at least about 5,000 different barcode sequences, at least about 10,000 different barcode sequences, at least about 50,000 different barcode sequences, at least about 100,000 different barcode sequences, at least about 1,000,000 different barcode sequences, at least about 5,000,000 different barcode sequences, or at least about 10,000,000 different barcode sequences, or more. Additionally, each bead can be provided with large numbers of nucleic acid (e.g., oligonucleotide) molecules attached. In particular, the number of molecules of nucleic acid molecules including the barcode sequence on an individual bead can be at least about 1,000 nucleic acid molecules, at least about 5,000 nucleic acid molecules, at least about 10,000 nucleic acid molecules, at least about 50,000 nucleic acid molecules, at least about 100,000 nucleic acid molecules, at least about 500,000 nucleic acids, at least about 1,000,000 nucleic acid molecules, at least about 5,000,000 nucleic acid molecules, at least about 10,000,000 nucleic acid molecules, at least about 50,000,000 nucleic acid molecules, at least about 100,000,000 nucleic acid molecules, at least about 250,000,000 nucleic acid molecules and in some cases at least about 1 billion nucleic acid molecules, or more. Nucleic acid molecules of a given bead can include identical (or common) barcode sequences, different barcode sequences, or a combination of both. Nucleic acid molecules of a given bead can include multiple sets of nucleic acid molecules. Nucleic acid molecules of a given set can include identical barcode sequences. The identical barcode sequences can be different from barcode sequences of nucleic acid molecules of another set. In some embodiments, such different barcode sequences can be associated with a given bead.

Moreover, when the population of beads is partitioned, the resulting population of partitions can also include a diverse barcode library that includes at least about 1,000 different barcode sequences, at least about 5,000 different barcode sequences, at least about 10,000 different barcode sequences, at least at least about 50,000 different barcode sequences, at least about 100,000 different barcode sequences, at least about 1,000,000 different barcode sequences, at least about 5,000,000 different barcode sequences, or at least about 10,000,000 different barcode sequences. In some cases, the population of beads provides a diverse barcode sequence library that includes about 1,000 to about 10,000 different barcode sequences, about 5,000 to about 50,000 different barcode sequences, about 10,000 to about 100,000 different barcode sequences, about 50,000 to about 1,000,000 different barcode sequences, or about 100,000 to about 10,000,000 different barcode sequences.

Additionally, each partition of the population can include at least about 1,000 nucleic acid molecules, at least about 5,000 nucleic acid molecules, at least about 10,000 nucleic acid molecules, at least about 50,000 nucleic acid molecules, at least about 100,000 nucleic acid molecules, at least about 500,000 nucleic acids, at least about 1,000,000 nucleic acid molecules, at least about 5,000,000 nucleic acid molecules, at least about 10,000,000 nucleic acid molecules, at least about 50,000,000 nucleic acid molecules, at least about 100,000,000 nucleic acid molecules, at least about 250,000,000 nucleic acid molecules and in some cases at least about 1 billion nucleic acid molecules.

In some cases, it may be desirable to incorporate multiple different barcodes within a given partition, either attached to a single or multiple beads within the partition. For example, in some cases, a mixed, but known set of barcode sequences can provide greater assurance of identification in the subsequent processing, e.g., by providing a stronger address or attribution of the barcodes to a given partition, as a duplicate or independent confirmation of the output from a given partition.

The nucleic acid molecules (e.g., oligonucleotides) are releasable from the beads upon the application of a particular stimulus to the beads. In some cases, the stimulus can be a photo-stimulus, e.g., through cleavage of a photo-labile linkage that releases the nucleic acid molecules. In other cases, a thermal stimulus can be used, where elevation of the temperature of the beads environment will result in cleavage of a linkage or other release of the nucleic acid molecules from the beads. In still other cases, a chemical stimulus can be used that cleaves a linkage of the nucleic acid molecules to the beads, or otherwise results in release of the nucleic acid molecules from the beads. In one case, such compositions include the polyacrylamide matrices described above for encapsulation of biological particles, and can be degraded for release of the attached nucleic acid molecules through exposure to a reducing agent, such as DTT.

### Systems and Methods for Controlled Partitioning

In some aspects, provided are systems and methods for controlled partitioning. Droplet size can be controlled by adjusting certain geometric features in channel architecture (e.g., microfluidics channel architecture). For example, an expansion angle, width, and/or length of a channel can be adjusted to control droplet size.

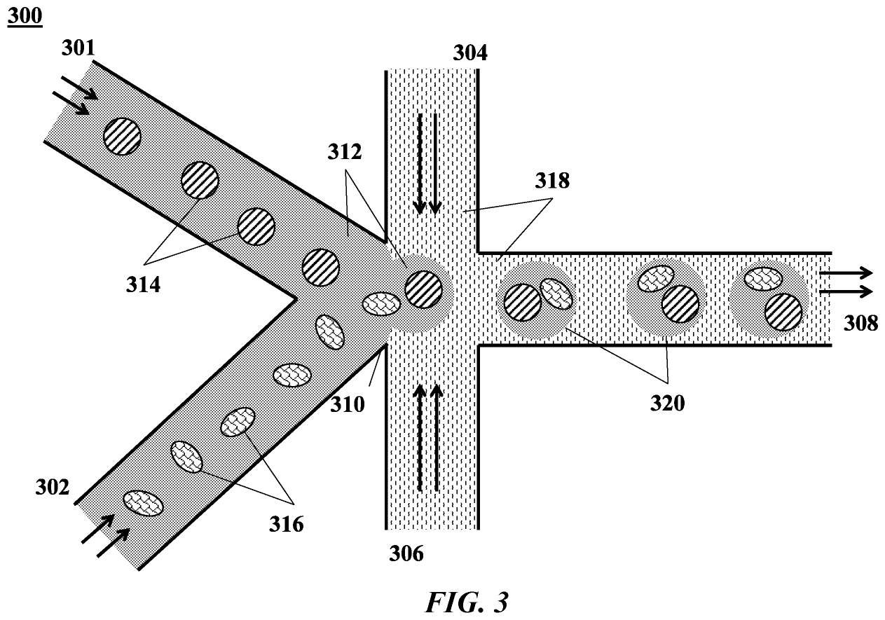

FIG. 3 shows an example of a microfluidic channel structure 300 for delivering barcode carrying beads to droplets. The channel structure 300 can include channel segments 301, 302, 304, 306 and 308 communicating at a channel junction 310. In operation, the channel segment 301 may transport an aqueous fluid 312 that includes a plurality of beads 314 (e.g., with nucleic acid molecules, oligonucleotides, molecular tags) along the channel segment 301 into junction 310. The plurality of beads 314 may be sourced from a suspension of beads. For example, the channel segment 301 may be connected to a reservoir comprising an aqueous suspension of beads 314. The channel segment 302 may transport the aqueous fluid 312 that includes a plurality of biological particles 316 along the channel segment 302 into junction 310. The plurality of biological particles 316 may be sourced from a suspension of biological particles. For example, the channel segment 302 may be connected to a reservoir comprising an aqueous suspension of biological particles 316. In some instances, the aqueous fluid 312 in either the first channel segment 301 or the second channel segment 302, or in both segments, can include one or more reagents, as further described below. A second fluid 318 that is immiscible with the aqueous fluid 312 (e.g., oil) can be delivered to the junction 310 from each of channel segments 304 and 306. Upon meeting of the aqueous fluid 312 from each of channel segments 301 and 302 and the second fluid 318 from each of channel segments 304 and 306 at the channel junction 310, the aqueous fluid 312 can be partitioned as discrete droplets 320 in the second fluid 318 and flow away from the junction 310 along channel segment 308. The channel segment 308 may deliver the discrete droplets to an outlet reservoir fluidly coupled to the channel segment 308, where they may be harvested. As an alternative, the channel segments 301 and 302 may meet at another junction upstream of the junction 310. At such junction, beads and biological particles may form a mixture that is directed along another channel to the junction 310 to yield droplets 320. The mixture may provide the beads and biological particles in an alternating fashion, such that, for example, a droplet comprises a single bead and a single biological particle.

FIG. 2 shows an example of a microfluidic channel structure for the controlled partitioning of beads into discrete droplets. A channel structure 200 can include a channel segment 202 communicating at a channel junction 206 (or intersection) with a reservoir 204. The reservoir 204 can be a chamber. Any reference to “reservoir,” as used herein, can also refer to a “chamber.” In operation, an aqueous fluid 208 that includes suspended beads 212 can be transported along the channel segment 202 into the junction 206 to meet a second fluid 210 that is immiscible with the aqueous fluid 208 in the reservoir 204 to create droplets 216, 218 of the aqueous fluid 208 flowing into the reservoir 204. At the junction 206 where the aqueous fluid 208 and the second fluid 210 meet, droplets can form based on factors such as the hydrodynamic forces at the junction 206, flow rates of the two fluids 208, 210, fluid properties, and certain geometric parameters (e.g., w, h0, α, etc.) of the channel structure 200. A plurality of droplets can be collected in the reservoir 204 by continuously injecting the aqueous fluid 208 from the channel segment 202 through the junction 206.

A discrete droplet generated can include a bead (e.g., as in occupied droplets 216). Alternatively, a discrete droplet generated can include more than one bead. Alternatively, a discrete droplet generated cannot include any beads (e.g., as in unoccupied droplet 218). In some instances, a discrete droplet generated can contain one or more biological particles, as described elsewhere herein. In some instances, a discrete droplet generated can include one or more reagents, as described elsewhere herein.

In some instances, the aqueous fluid 208 can have a substantially uniform concentration or frequency of beads 212. The beads 212 can be introduced into the channel segment 202 from a separate channel (not shown in FIG. 2). The frequency of beads 212 in the channel segment 202 can be controlled by controlling the frequency in which the beads 212 are introduced into the channel segment 202 and/or the relative flow rates of the fluids in the channel segment 202 and the separate channel. In some instances, the beads can be introduced into the channel segment 202 from a plurality of different channels, and the frequency controlled accordingly.

In some instances, the aqueous fluid 208 in the channel segment 202 can include biological particles (e.g., described with reference to FIG. 1). In some instances, the aqueous fluid 208 can have a substantially uniform concentration or frequency of biological particles. As with the beads, the biological particles (e.g., labelled cells, human cells or other cells expressing a variant antibody or variant antigen-binding fragment thereof) can be introduced into the channel segment 202 from a separate channel. The frequency or concentration of the biological particles in the aqueous fluid 208 in the channel segment 202 can be controlled by controlling the frequency in which the biological particles are introduced into the channel segment 202 and/or the relative flow rates of the fluids in the channel segment 202 and the separate channel. In some instances, the biological particles can be introduced into the channel segment 202 from a plurality of different channels, and the frequency controlled accordingly. In some instances, a first separate channel can introduce beads and a second separate channel can introduce biological particles into the channel segment 202. The first separate channel introducing the beads can be upstream or downstream of the second separate channel introducing the biological particles.

The second fluid 210 can include an oil, such as a fluorinated oil, that includes a fluorosurfactant for stabilizing the resulting droplets, for example, inhibiting subsequent coalescence of the resulting droplets.

In some instances, the second fluid 210 cannot be subjected to and/or directed to any flow in or out of the reservoir 204. For example, the second fluid 210 can be substantially stationary in the reservoir 204. In some instances, the second fluid 210 can be subjected to flow within the reservoir 204, but not in or out of the reservoir 204, such as via application of pressure to the reservoir 204 and/or as affected by the incoming flow of the aqueous fluid 208 at the junction 206. Alternatively, the second fluid 210 can be subjected and/or directed to flow in or out of the reservoir 204. For example, the reservoir 204 can be a channel directing the second fluid 210 from upstream to downstream, transporting the generated droplets.

Systems and methods for controlled partitioning are described further in PCT/US2018/047551, which is hereby incorporated by reference in its entirety.

Subsequent operations that can be performed can include generation of amplification products, purification (e.g., via solid phase reversible immobilization (SPRI)), further processing (e.g., shearing, ligation of functional sequences, and subsequent amplification (e.g., via PCR)). These operations can occur in bulk (e.g., outside the partition). In the case where a partition is a droplet in an emulsion, the emulsion can be broken and the contents of the droplet pooled for additional operations. Additional reagents that can be co-partitioned along with the barcode bearing bead can include oligonucleotides to block ribosomal RNA (rRNA) and nucleases to digest genomic DNA from cells. Alternatively, rRNA removal agents can be applied during additional processing operations. The configuration of the constructs generated by such a method can help minimize (or avoid) sequencing of the poly-T sequence during sequencing and/or sequence the 5′ end of a polynucleotide sequence. The amplification products, for example, first amplification products and/or second amplification products, can be subject to sequencing for sequence analysis. In some cases, amplification can be performed using the Partial Hairpin Amplification for Sequencing (PHASE) method.

A variety of applications require the evaluation of the presence and quantification of different biological particle or organism types within a population of biological particles, including, for example, microbiome analysis and characterization, environmental testing, food safety testing, epidemiological analysis, e.g., in tracing contamination or the like.

Partitions including a barcode bead (e.g., a gel bead) associated with barcode molecules and a bead encapsulating cellular constituents (e.g., a cell bead) such as cellular nucleic acids can be useful in constituent analysis as is described in U.S. Patent Publication No. 2018/0216162.

### Sample and Cell Processing

In methods described herein, nucleic acids encoding a selected antibody, or selected antigen-binding fragment thereof, may have been derived from cells of any organism capable to expressing, or being induced to express an antibody, or antigen-binding fragment thereof. For instance, the nucleic acids encoding the selected antibody, or selected antigen-binding fragment thereof, may have been derived from cells of a donor, such as a human donor, or a mouse. The nucleic acids encoding the selected antibody, or selected antigen-binding fragment thereof, may have been derived from cells of multiple donors, such as multiple human donors or mice. The cells obtained from the donor, donors, mouse, mice or other organism(s), may have been of a sample or samples acquired from the donor, donors, mouse, mice or other organism(s).

A sample containing the cell or cells, or cellular nuclei from which the nucleic acids encoding the selected antibody, or selected antigen-binding fragment thereof, is derived may have been obtained from one or more biological samples, each including one or more biological particles, such as one or more cells and/or cellular constituents, such as one or more cell nuclei. For example, the sample or samples may include a plurality of cells and/or cellular constituents. Components (e.g., cells or cellular constituents, such as cell nuclei) of a sample or samples can be of a single type or a plurality of different types. For example, if a sample or samples includes more than one cell the cell can include one or more different types of blood cells.

The sample or samples containing the cell or cells from which the nucleic acids encoding the selected antibody, or selected antigen-binding fragment thereof, is derived may one include a plurality of cells having different dimensions and features. In some cases, processing of the sample or samples, such as by cell separation and sorting (e.g., as described herein), can affect the distribution of dimensions and cellular features included in the sample(s) by depleting cells having certain features and dimensions and/or isolating cells having certain features and dimensions to obtain those cells which contain nucleic acid sequences of potentially selected antibodies or antigen-binding fragments thereof.

A sample or samples may undergo one or more processes in preparation for analysis, and/or deriving, of the nucleic acid sequence encoding the selected antibody, or selected antigen-binding fragment thereof (e.g., as described herein), including, but not limited to, filtration, selective precipitation, purification, centrifugation, permeabilization, isolation, agitation, heating, and/or other processes. For example, a sample may be filtered to remove a contaminant or other materials. In an example, a filtration process can include the use of microfluidics (e.g., to separate biological particles of different sizes, types, charges, or other features).

In an example, a sample or samples including one or more cells can be processed to separate the one or more cells from other materials in the sample(s) (e.g., using centrifugation and/or another process). In some cases, cells and/or cellular constituents of a sample or samples can be processed to separate and/or sort groups of cells and/or cellular constituents, such as to separate and/or sort cells and/or cellular constituents of different types. Examples of cell separation include, but are not limited to, separation of white blood cells or immune cells from other blood cells and components, separation of circulating tumor cells from blood, and separation of bacteria from bodily cells and/or environmental materials. A separation process can include a positive selection process (e.g., targeting of a cell type of interest for retention for subsequent downstream analysis, such as by use of a monoclonal antibody that targets a surface marker of the cell type of interest), a negative selection process (e.g., removal of one or more cell types and retention of one or more other cell types of interest), and/or a depletion process (e.g., removal of a single cell type from a sample, such as removal of red blood cells from peripheral blood mononuclear cells).

Separation of one or more different types of cells can include, for example, centrifugation, filtration, microfluidic-based sorting, flow cytometry, fluorescence-activated cell sorting (FACS), magnetic-activated cell sorting (MACS), buoyancy-activated cell sorting (BACS), or any other useful method. For example, a flow cytometry method can be used to detect cells and/or cellular constituents based on a parameter such as a size, morphology, or protein expression. Flow cytometry-based cell sorting can include injecting a sample into a sheath fluid that conveys the cells and/or cellular constituents of the sample into a measurement region one at a time. In the measurement region, a light source such as a laser can interrogate the cells and/or cellular constituents and scattered light and/or fluorescence can be detected and converted into digital signals. A nozzle system (e.g., a vibrating nozzle system) can be used to generate droplets (e.g., aqueous droplets) including individual cells and/or cellular constituents. Droplets including cells and/or cellular constituents of interest (e.g., as determined via optical detection) can be labeled with an electric charge (e.g., using an electrical charging ring), which charge can be used to separate such droplets from droplets including other cells and/or cellular constituents. For example, FACS can include labeling cells and/or cellular constituents with fluorescent markers (e.g., using internal and/or external biomarkers). Cells and/or cellular constituents can then be measured and identified one by one and sorted based on the emitted fluorescence of the marker or absence thereof. MACS can use micro- or nano-scale magnetic particles to bind to cells and/or cellular constituents (e.g., via an antibody interaction with cell surface markers) to facilitate magnetic isolation of cells and/or cellular constituents of interest from other components of a sample (e.g., using a column-based analysis). BACS can use microbubbles (e.g., glass microbubbles) labeled with antibodies to target cells of interest. Cells and/or cellular components coupled to microbubbles can float to a surface of a solution, thereby separating target cells and/or cellular components from other components of a sample. Cell separation techniques can be used to enrich for populations of cells of interest. For example, a sample including a plurality of cells of a given type can be subjected to a positive separation process. The plurality of cells of the given type can be labeled with a fluorescent marker (e.g., based on an expressed cell surface marker or another marker) and subjected to a FACS process to separate these cells from other cells of the plurality of cells. The selected cells can then be subjected to subsequent partition-based analysis (e.g., as described herein) or other downstream analysis. The fluorescent marker can be removed prior to such analysis or can be retained. The fluorescent marker can include an identifying feature, such as a nucleic acid barcode sequence and/or unique molecular identifier.

In another example, a first sample including a first plurality of cells including a first plurality of cells of a given type (e.g., immune cells, such as B cells, expressing a particular marker or combination of markers) and a second sample including a second plurality of cells including a second plurality of cells of the given type can be subjected to a positive separation process. The first and second samples can be collected from the same or different subjects, e.g., donors, at the same or different types, from the same or different bodily locations or systems, using the same or different collection techniques. For example, the first sample can be from a first subject, e.g., donor, and the second sample can be from a second subject, e.g., donor, different than the first subject, e.g., donor. The first plurality of cells of the first sample can be provided a first plurality of fluorescent markers configured to label the first plurality of cells of the given type, e.g., B cells. The second plurality of cells of the second sample can be provided a second plurality of fluorescent markers configured to label the second plurality of cells of the given type, e.g., B cells. The first plurality of fluorescent markers can include a first identifying feature, such as a first barcode, while the second plurality of fluorescent markers can include a second identifying feature, such as a second barcode, that is different than the first identifying feature. The first plurality of fluorescent markers and the second plurality of fluorescent markers can fluoresce at the same intensities and over the same range of wavelengths upon excitation with a same excitation source (e.g., light source, such as a laser). The first and second samples can then be combined and subjected to a FACS process to separate cells of the given type, e.g., B cells, from other cells based on the first plurality of fluorescent markers labeling the first plurality of cells of the given type, e.g., B cells, and the second plurality of fluorescent markers labeling the second plurality of cells of the given type. Alternatively, the first and second samples can undergo separate FACS processes and the positively selected cells of the given type, e.g., B cells, from the first sample and the positively selected cells of the given type, e.g., B cells, from the second sample can then be combined for subsequent analysis. The encoded identifying features of the different fluorescent markers can be used to identify cells originating from the first sample and cells originating from the second sample. For example, the first and second identifying features can be configured to interact (e.g., in partitions, as described herein) with nucleic acid barcode molecules (e.g., as described herein) to generate barcoded nucleic acid products detectable using, e.g., nucleic acid sequencing.

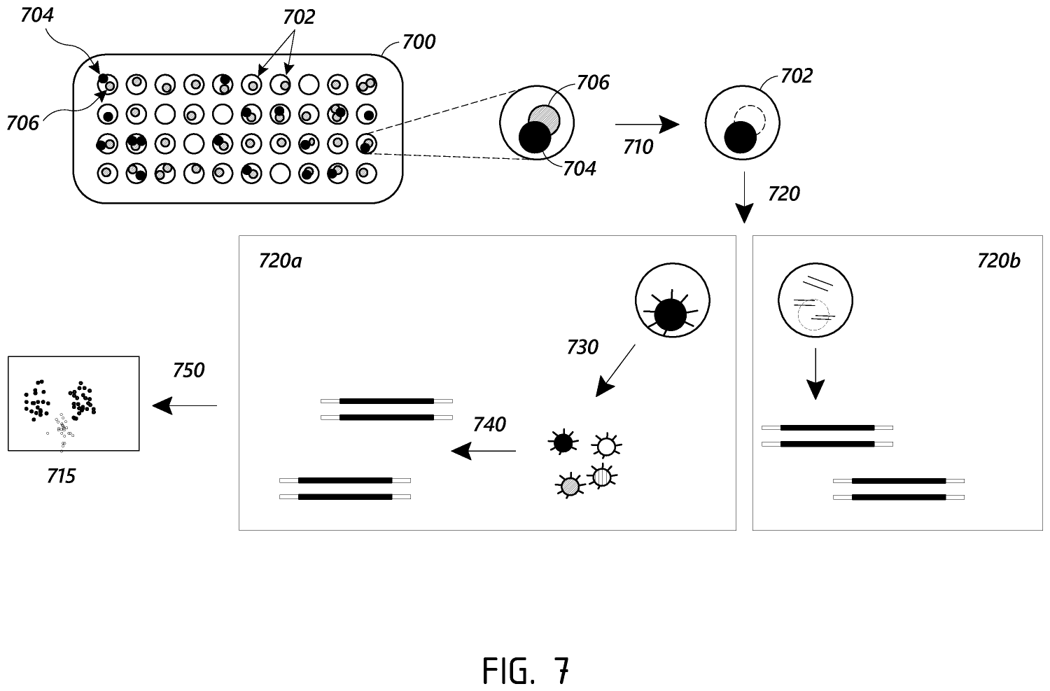

FIG. 7 schematically shows an example workflow for processing nucleic acid molecules within a sample, e.g., for obtaining a nucleic acid sequence encoding an antibody, or antigen-binding fragment thereof. A substrate 700 including a plurality of microwells 702 can be provided. A sample 706 which can include a cell, cell bead, cellular components or analytes (e.g., proteins and/or nucleic acid molecules) can be co-partitioned, in a plurality of microwells 702, with a plurality of beads 704 including nucleic acid barcode molecules. During a partitioning process, the sample 706 can be processed within the partition. For instance, in the case of live cells, the cell can be subjected to conditions sufficient to lyse the cells and release the analytes contained therein. In process 720, the bead 704 can be further processed. By way of example, processes 720a and 720b schematically illustrate different workflows, depending on the properties of the bead 704.

In 720a, the bead includes nucleic acid barcode molecules that are attached thereto, and sample nucleic acid molecules (e.g., RNA, DNA) can attach, e.g., via hybridization of ligation, to the nucleic acid barcode molecules. Such attachment can occur on the bead. In process 730, the beads 704 from multiple wells 702 can be collected and pooled. Further processing can be performed in process 740. For example, one or more nucleic acid reactions can be performed, such as reverse transcription, nucleic acid extension, amplification, ligation, transposition, etc. In some instances, adapter sequences are ligated to the nucleic acid molecules, or derivatives thereof, as described elsewhere herein. For instance, sequencing primer sequences can be appended to each end of the nucleic acid molecule. In process 750, further characterization, such as sequencing can be performed to generate sequencing reads. The sequencing reads can yield information on individual cells or populations of cells, which can be represented visually or graphically, e.g., in a plot.

In 720b, the bead includes nucleic acid barcode molecules that are releasably attached thereto, as described below. The bead can degrade or otherwise release the nucleic acid barcode molecules into the well 702; the nucleic acid barcode molecules can then be used to barcode nucleic acid molecules within the well 702. Further processing can be performed either inside the partition or outside the partition. For example, one or more nucleic acid reactions can be performed, such as reverse transcription, nucleic acid extension, amplification, ligation, transposition, etc. In some instances, adapter sequences are ligated to the nucleic acid molecules, or derivatives thereof, as described elsewhere herein. For instance, sequencing primer sequences can be appended to each end of the nucleic acid molecule. In process 750, further characterization, such as sequencing can be performed to generate sequencing reads. The sequencing reads can yield information on individual cells or populations of cells, which can be represented visually or graphically, e.g., in a plot.

Multiplexing Methods

In some embodiments of the disclosure, certain steps of the methods described herein are performed in multiplex format. For example, in some embodiments, a step of partitioning is performed. The step of partitioning can include individually partitioning additional single cells of the plurality of cells in additional partitions of the plurality of partitions. Furthermore, the step of generating barcoded nucleic acid molecules can be performed in multiplex across partitions of the plurality of partitions, and for analyzing more than one analyte in partitions of the plurality of partitions.

Accordingly, in some embodiments, the present disclosure provides methods and systems for multiplexing, and otherwise increasing throughput of samples for analysis. For example, a single or integrated process workflow may permit the processing, identification, and/or analysis of one or more of multiple analytes, and analyte characterizations. For example, in the methods and systems described herein, one or more labelling agents capable of binding to or otherwise coupling to one or more cells or cell features can be used to characterize cells and/or cell features. In some instances, cell features include cell surface features. Cell surface features can include, but are not limited to, a receptor, an antigen or antigen fragment, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, a B-cell receptor (e.g., that binds a target antigen), a chimeric antigen receptor, a gap junction, an adherens junction, or any combination thereof. In some instances, cell features can include intracellular analytes, such as proteins, protein modifications (e.g., phosphorylation status or other post-translational modifications), nuclear proteins, nuclear membrane proteins, or any combination thereof. A labelling agent can include, but is not limited to, a protein, a peptide, an antibody (or an epitope binding fragment thereof), an antigen (e.g., target antigen), an antigen fragment (e.g., fragment of the target antigen), a lipophilic moiety (such as cholesterol), a cell surface receptor binding molecule, a receptor ligand, a small molecule, a bi-specific antibody, a B-cell receptor engager, a pro-body, an aptamer, a monobody, an affimer, a Darpin, and a protein scaffold, or any combination thereof. The labelling agents can include (e.g., are attached to) a reporter oligonucleotide that is indicative of the cell surface feature to which the binding group binds. For example, the reporter oligonucleotide can include a barcode sequence that permits identification of the labelling agent. For example, a labelling agent that is specific to one type of cell feature (e.g., a first cell surface feature) can have a first reporter oligonucleotide coupled thereto, while a labelling agent that is specific to a different cell feature (e.g., a second cell surface feature) can have a different reporter oligonucleotide coupled thereto. For a description of exemplary labelling agents, reporter oligonucleotides, and methods of use, see, e.g., U.S. Pat. No. 10,550,429; U.S. Pat. Pub. 20190177800; and U.S. Pat. Pub. 20190367969.

In a particular example, a library of potential cell feature labelling agents can be provided, where the respective cell feature labelling agents are associated with nucleic acid reporter molecules, such that a different reporter oligonucleotide sequence is associated with each labelling agent capable of binding to a specific cell feature. In some embodiments, the cell feature labelling agents comprise a target antigen, as disclosed herein. In other aspects, different members of the library can be characterized by the presence of a different oligonucleotide sequence label. For example, a B cell receptor capable of binding to a target antigen can have associated with it a first reporter oligonucleotide sequence. The presence of the particular oligonucleotide sequence(s) can be indicative of the presence of a particular cell feature, e.g., B cell receptor, which can recognize or bind the particular target antigen.

Labelling agents capable of binding to or otherwise coupling to one or more cells can be used to characterize a cell as belonging to a particular set of cells, e.g., B cells from a donor. For example, labelling agents can be used to label a sample of cells, e.g., to provide a sample index. For another example, labelling agents can be used to label a group of cells belonging to a particular experimental condition. In this way, a group of cells can be labeled as different from another group of cells. In an example, a first group of cells can originate from a first sample, e.g., of a first donor, and a second group of cells can originate from a second sample, e.g., of a second donor. Labelling agents can allow the first group and second group to have a different labelling agent (or reporter oligonucleotide associated with the labelling agent). This can, for example, facilitate multiplexing, where cells of the first group and cells of the second group can be labeled separately and then pooled together for downstream analysis. The downstream detection of a label can indicate analytes as belonging to a particular group.

For example, a reporter oligonucleotide can be linked to a target antigen, and labeling a cell can include subjecting the target antigen-linked barcode molecule to conditions suitable for binding the target antigen or fragment thereof to a molecule present on a surface of the cell, e.g., a B cell receptor. The binding affinity between the target antigen and the molecule present on the surface (e.g., B cell receptor or immunoglobulin) can be within a desired range to ensure that the target antigen remains bound to the molecule, e.g., B cell receptor. For example, the binding affinity can be within a desired range to ensure that the B cell receptor remains bound to the target antigen during various sample processing steps, such as partitioning and/or nucleic acid amplification or extension. A dissociation constant (Kd) between the target antigen and the molecule to which it binds, e.g., B cell receptor, can be less than about 100 μM, 90 μM, 80 μM, 70 μM, 60 μM, 50 μM, 40 μM, 30 μM, 20 μM, 10 μM, 9 μM, 8 μM, 7 μM, 6 μM, 5 μM, 4 μM, 3 M, 2 μM, 1 μM, 900 nM, 800 nM, 700 nM, 600 nM, 500 nM, 400 nM, 300 nM, 200 nM, 100 nM, 90 nM, 80 nM, 70 nM, 60 nM, 50 nM, 40 nM, 30 nM, 20 nM, 10 nM, 9 nM, 8 nM, 7 nM, 6 nM, 5 nM, 4 nM, 3 nM, 2 nM, 1 nM, 900 μM, 800 μM, 700 μM, 600 μM, 500 μM, 400 μM, 300 μM, 200 μM, 100 μM, 90 μM, 80 μM, 70 μM, 60 μM, 50 μM, 40 μM, 30 μM, 20 μM, 10 μM, 9 μM, 8 μM, 7 μM, 6 μM, 5 μM, 4 μM, 3 μM, 2 μM, or 1 μM. For example, the dissociation constant can be less than about 10 μM. In some embodiments, the interaction has a desired off rate (koff), such that the target antigen or fragment thereof and its binding partner, e.g., B cell receptor, remain bound to during various sample processing steps.

In another example, a cell may be, or may further be, incubated in the presence of a reporter oligonucleotide that can be coupled to a cell-penetrating peptide (CPP), and the cells can be labeled by delivering the CPP coupled reporter oligonucleotide into a biological particle. Labeling biological particles can include delivering the CPP conjugated oligonucleotide into a cell and/or cell bead by the cell-penetrating peptide. A CPP that can be used in the methods provided herein can include at least one non-functional cysteine residue, which can be either free or derivatized to form a disulfide link with an oligonucleotide that has been modified for such linkage. Non-limiting examples of CPPs that can be used in embodiments herein include penetratin, transportan, pls1, TAT(48-60), pVEC, MTS, and MAP. Cell-penetrating peptides useful in the methods provided herein can have the capability of inducing cell penetration for at least about 30%, 40%, 50%, 60%, 70%, 80%, 90%, 95%, 96%, 97%, 98%, 99%, or 100% of cells of a cell population. The CPP can be an arginine-rich peptide transporter. The CPP can be Penetratin or the Tat peptide. In another example, a reporter oligonucleotide can be coupled to a fluorophore or dye, and labeling cells can include subjecting the fluorophore-linked barcode molecule to conditions suitable for binding the fluorophore to the surface of the cell. In some instances, fluorophores can interact strongly with lipid bilayers and labeling cells can include subjecting the fluorophore-linked barcode molecule to conditions such that the fluorophore binds to or is inserted into a membrane of the cell. In some cases, the fluorophore is a water-soluble, organic fluorophore. In some instances, the fluorophore is Alexa 532 maleimide, tetramethylrhodamine-5-maleimide (TMR maleimide), BODIPY-TMR maleimide, Sulfo-Cy3 maleimide, Alexa 546 carboxylic acid/succinimidyl ester, Atto 550 maleimide, Cy3 carboxylic acid/succinimidyl ester, Cy3B carboxylic acid/succinimidyl ester, Atto 565 biotin, Sulforhodamine B, Alexa 594 maleimide, Texas Red maleimide, Alexa 633 maleimide, Abberior STAR 635P azide, Atto 647N maleimide, Atto 647 SE, or Sulfo-Cy5 maleimide. See, e.g., Hughes L D, et al. PLoS One. 2014 Feb. 4; 9(2):e87649 for a description of organic fluorophores.

A reporter oligonucleotide can be coupled to a lipophilic molecule, and labeling cells can include delivering the nucleic acid barcode molecule to a membrane of a cell or a nuclear membrane by the lipophilic molecule. Lipophilic molecules can associate with and/or insert into lipid membranes such as cell membranes and nuclear membranes. In some cases, the insertion can be reversible. In some cases, the association between the lipophilic molecule and the cell or nuclear membrane can be such that the membrane retains the lipophilic molecule (e.g., and associated components, such as nucleic acid barcode molecules, thereof) during subsequent processing (e.g., partitioning, cell permeabilization, amplification, pooling, etc.). The reporter nucleotide can enter into the intracellular space and/or a cell nucleus. In some embodiments, a reporter oligonucleotide coupled to a lipophilic molecule will remain associated with and/or inserted into lipid membrane (as described herein) via the lipophilic molecule until lysis of the cell occurs, e.g., inside a partition. Exemplary embodiments of lipophilic molecules coupled to reporter oligonucleotides are described in PCT/US2018/064600.

A reporter oligonucleotide can be part of a nucleic acid molecule including any number of functional sequences, as described elsewhere herein, such as a target capture sequence, a random primer sequence, and the like, and coupled to another nucleic acid molecule that is, or is derived from, the analyte.

Prior to providing the nucleic acid sequence, or (if comprised in the method) a step of partitioning, the cells can be incubated with the library of labelling agents, that can be labelling agents to a broad panel of different cell features, e.g., receptors, proteins, etc., and which include their associated reporter oligonucleotides. Unbound labelling agents can be washed from the cells, and the cells can then be co-partitioned (e.g., into droplets or wells) along with partition-specific barcode oligonucleotides (e.g., attached to a support, such as a bead or gel bead) as described elsewhere herein. As a result, the partitions can include the cell or cells, as well as the bound labelling agents and their known, associated reporter oligonucleotides.

In other instances, e.g., to facilitate sample multiplexing, a labelling agent that is specific to a particular cell feature can have a first plurality of the labelling agent (e.g., an antibody or lipophilic moiety) coupled to a first reporter oligonucleotide and a second plurality of the labelling agent coupled to a second reporter oligonucleotide. For example, the first plurality of the labelling agent and second plurality of the labelling agent can interact with different cells, cell populations or samples, allowing a particular report oligonucleotide to indicate a particular cell population (or cell or sample) and cell feature. In this way, different samples or groups can be independently processed and subsequently combined together for pooled analysis (e.g., partition-based barcoding as described elsewhere herein). See, e.g., U.S. Pat. Pub. 20190323088.

In some embodiments, to facilitate sample multiplexing, individual samples can be stained with lipid tags, such as cholesterol-modified oligonucleotides (CMOs), anti-calcium channel antibodies, or anti-ACTB antibodies. Non-limiting examples of anti-calcium channel antibodies include anti-KCNN4 antibodies, anti-BK channel beta 3 antibodies, anti-a1B calcium channel antibodies, and anti-CACNA1A antibodies. Examples of anti-ACTB antibodies suitable for the methods of the disclosure include, but are not limited to, mAbGEa, ACTN05, AC-15, 15G5A11/E2, BA3R, and HHF35.

As described elsewhere herein, libraries of labelling agents can be associated with a particular cell feature as well as be used to identify analytes as originating from a particular cell population, or sample. Cell populations can be incubated with a plurality of libraries such that a cell or cells include multiple labelling agents. For example, a cell can include coupled thereto a lipophilic labelling agent and an antibody. The lipophilic labelling agent can indicate that the cell is a member of a particular cell sample, whereas the antibody can indicate that the cell includes a particular analyte. In this manner, the reporter oligonucleotides and labelling agents can allow multi-analyte, multiplexed analyses to be performed.

In some instances, these reporter oligonucleotides can include nucleic acid barcode sequences that permit identification of the labelling agent which the reporter oligonucleotide is coupled to. The use of oligonucleotides as the reporter can provide advantages of being able to generate significant diversity in terms of sequence, while also being readily attachable to most biomolecules, e.g., antibodies, etc., as well as being readily detected, e.g., using sequencing or array technologies.

Attachment (coupling) of the reporter oligonucleotides to the labelling agents, e.g., a target antigen or fragment thereof, can be achieved through any of a variety of direct or indirect, covalent or non-covalent associations or attachments. For example, reporter oligonucleotides can be covalently attached to a portion of a labelling agent (such a protein, e.g., a target antigen or target antigen fragment) using chemical conjugation techniques (e.g., Lightning-Link® antibody labelling kits available from Innova Biosciences), as well as other non-covalent attachment mechanisms, e.g., using biotinylated antibodies (or biotinylated antigens, or biotinylated antigen fragments) and oligonucleotides (or beads that include one or more biotinylated linker, coupled to oligonucleotides) with an avidin or streptavidin linker. Antibody and oligonucleotide biotinylation techniques are available. See, e.g., Fang, et al., “Fluoride-Cleavable Biotinylation Phosphoramidite for 5′-end-Labelling and Affinity Purification of Synthetic Oligonucleotides,” Nucleic Acids Res. Jan. 15, 2003; 31(2):708-715. Likewise, protein and peptide biotinylation techniques have been developed and are readily available. See, e.g., U.S. Pat. No. 6,265,552. Furthermore, click reaction chemistry such as a Methyltetrazine-PEG5-NHS Ester reaction, a TCO-PEG4-NHS Ester reaction, or the like, can be used to couple reporter oligonucleotides to labelling agents. Commercially available kits, such as those from Thunderlink and Abcam, and techniques common in the art can be used to couple reporter oligonucleotides to labelling agents as appropriate. In another example, a labelling agent is indirectly (e.g., via hybridization) coupled to a reporter oligonucleotide including a barcode sequence that identifies the label agent. For instance, the labelling agent can be directly coupled (e.g., covalently bound) to a hybridization oligonucleotide that includes a sequence that hybridizes with a sequence of the reporter oligonucleotide. Hybridization of the hybridization oligonucleotide to the reporter oligonucleotide couples the labelling agent to the reporter oligonucleotide. In some embodiments, the reporter oligonucleotides are releasable from the labelling agent, such as upon application of a stimulus. For example, the reporter oligonucleotide can be attached to the labelling agent through a labile bond (e.g., chemically labile, photolabile, thermally labile, etc.) as generally described for releasing molecules from supports elsewhere herein. In some instances, the reporter oligonucleotides described herein can include one or more functional sequences that can be used in subsequent processing, such as an adapter sequence, a unique molecular identifier (UMI) sequence, a sequencer specific flow cell attachment sequence (such as an P5, P7, or partial P5 or P7 sequence), a primer or primer binding sequence, a sequencing primer or primer biding sequence (such as an R1, R2, or partial R1 or R2 sequence).

In some cases, the labelling agent (e.g., a target antigen, a target antigen fragment) is presented as a monomer. In some cases, the labelling agent is presented as a multimer. In some cases, a labelling agent (e.g., a target antigen, a target antigen fragment) is presented as a dimer. In some cases, a labelling agent (e.g., a target antigen, a target antigen fragment,) is presented as a trimer. In some cases, a labelling agent (e.g., a target antigen, target antigen fragment) is presented as a tetramer. In some cases, a labelling agent (e.g., a target antigen, a target antigen fragment) is presented as a pentamer. In some cases, a labelling agent (e.g., a target antigen, a target antigen fragment) is presented as a hexamer. In some cases, a labelling agent (e.g., a target antigen, a target antigen fragment) is presented as a heptamer. In some cases, a labelling agent (e.g., a target antigen, a target antigen fragment) is presented as an octamer. In some cases, a labelling agent (e.g., a target antigen, a target antigen fragment) is presented as a nonamer. In some cases, a labelling agent (e.g., a target antigen, a target antigen fragment) is presented as a decamer. In some cases, a labelling agent (e.g., a target antigen, a target antigen fragment) is presented as a 10+-mer.

In some cases, the labelling agent can include a reporter oligonucleotide and a label. A label can be fluorophore, a radioisotope, a molecule capable of a colorimetric reaction, a magnetic particle, or any other suitable molecule or compound capable of detection. The label can be conjugated to a labelling agent (or reporter oligonucleotide) either directly or indirectly (e.g., the label can be conjugated to a molecule that can bind to the labelling agent or reporter oligonucleotide). In some cases, a label is conjugated to an oligonucleotide that is complementary to a sequence of the reporter oligonucleotide, and the oligonucleotide can be allowed to hybridize to the reporter oligonucleotide.

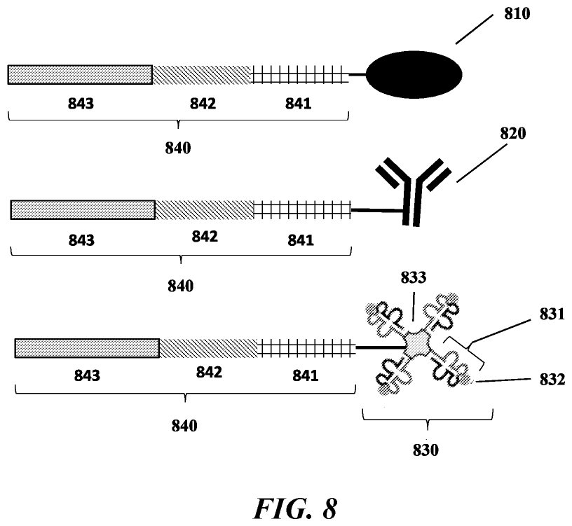

FIG. 8 describes exemplary labelling agents (810, 820, 830) including reporter oligonucleotides (840) attached thereto. Labelling agent 810 (e.g., any of the labelling agents described herein) is attached (either directly, e.g., covalently attached, or indirectly) to reporter oligonucleotide 840. Reporter oligonucleotide 840 can include barcode sequence 842 that identifies labelling agent 810. Reporter oligonucleotide 840 can also include one or more functional sequences 843 that can be used in subsequent processing, such as an adapter sequence, a unique molecular identifier (UMI) sequence, a sequencer specific flow cell attachment sequence (such as an P5, P7, or partial P5 or P7 sequence), a primer or primer binding sequence, or a sequencing primer or primer binding sequence (such as an R1, R2, or partial R1 or R2 sequence).

Referring to FIG. 8, in some instances, reporter oligonucleotide 840 conjugated to a labelling agent (e.g., 810, 820, 830) includes a functional sequence 841, a reporter barcode sequence 842 that identifies the labelling agent (e.g., 810, 820, 830), and reporter capture handle 843. Reporter capture handle sequence 843 can be configured to hybridize to a complementary sequence, such as a complementary sequence present on a nucleic acid barcode molecule 890 (not shown), such as those described elsewhere herein. In some instances, nucleic acid barcode molecule 890 is attached to a support (e.g., a bead, such as a gel bead), such as those described elsewhere herein. For example, nucleic acid barcode molecule 890 can be attached to the support via a releasable linkage (e.g., including a labile bond), such as those described elsewhere herein. In some instances, reporter oligonucleotide 840 includes one or more additional functional sequences, such as those described above.

In some instances, the labelling agent 810 is a protein or polypeptide (e.g., a target antigen or prospective antigen, or a fragment of a target antigen or prospective antigen) including reporter oligonucleotide 840. Reporter oligonucleotide 840 includes reporter barcode sequence 842 that identifies polypeptide 810 and can be used to infer the presence of an analyte, e.g., a binding partner of polypeptide 810 (i.e., a molecule or compound to which polypeptide 810 can bind). In some instances, the labelling agent 810 is a lipophilic moiety (e.g., cholesterol) including reporter oligonucleotide 840, where the lipophilic moiety is selected such that labelling agent 810 integrates into a membrane of a cell or nucleus. Reporter oligonucleotide 840 includes reporter barcode sequence 842 that identifies lipophilic moiety 810 which in some instances is used to tag cells (e.g., groups of cells, cell samples, etc.) and can be used for multiplex analyses as described elsewhere herein. In some instances, the labelling agent is an antibody 820 (or an epitope binding fragment thereof) including reporter oligonucleotide 840. Reporter oligonucleotide 840 includes reporter barcode sequence 842 that identifies antibody 820 and can be used to infer the presence of, e.g., a target of antibody 820 (e.g., a molecule or compound to which antibody 820 binds). In other embodiments, labelling agent 830 includes an MHC molecule 831 including peptide 832 and reporter oligonucleotide 840 that identifies peptide 832. In some instances, the MHC molecule is coupled to a support 833. In some instances, support 833 can be or comprise a polypeptide, such as avidin, neutravidin, streptavidin, or a polysaccharide, such as dextran. In some embodiments, support 833 further comprises a detectable label, e.g., a detectable label described herein, e.g., a fluorescent label. In some instances, reporter oligonucleotide 840 can be directly or indirectly coupled to MHC labelling agent 830 in any suitable manner. For example, reporter oligonucleotide 0840 can be coupled to MHC molecule 831, support 833, or peptide 832. In some embodiments, labelling agent 830 includes a plurality of MHC molecules, (e.g. is an MHC multimer, which can be coupled to a support (e.g., 833)). In some embodiments, reporter oligonucleotide 840 and MHC molecule 830 are attached to the polypeptide or polysaccharide of support 833. In some embodiments, reporter oligonucleotide 840 and MHC molecule 830 are attached to the detectable label of support 833. In some embodiments, reporter oligonucleotide 840 and an antigen (e.g., protein, polypeptide, disclosed herein) are attached to polypeptide or polysaccharide of support 833. In some embodiments, reporter oligonucleotide 840 and an antigen (e.g., protein, polypeptide, disclosed herein) are attached to the detectable label of support 833. There are many possible configurations of Class I and/or Class II MHC multimers that can be utilized with the compositions, methods, and systems disclosed herein, e.g., MHC tetramers, MHC pentamers (MHC assembled via a coiled-coil domain, e.g., Pro5® MHC Class I Pentamers, (ProImmune, Ltd.), MHC octamers, MHC dodecamers, MHC decorated dextran molecules (e.g., MHC Dextramer® (Immudex)), etc. For a description of exemplary labelling agents, including antibody and MHC-based labelling agents, reporter oligonucleotides, and methods of use, see, e.g., U.S. Pat. No. 10,550,429 and U.S. Pat. Pub. 20190367969.

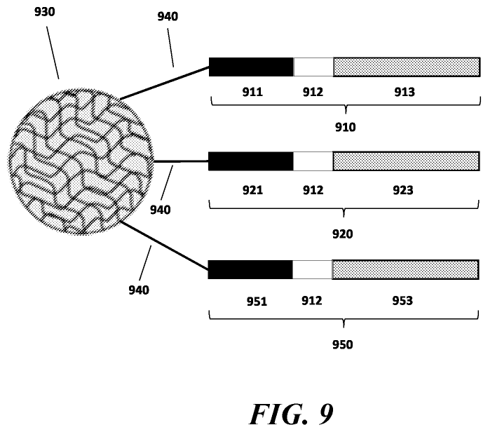

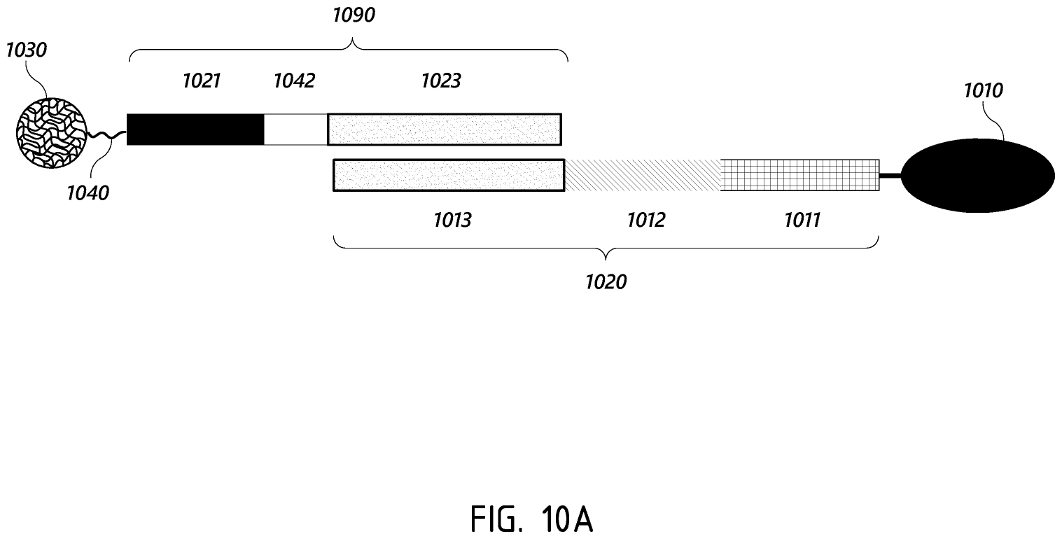

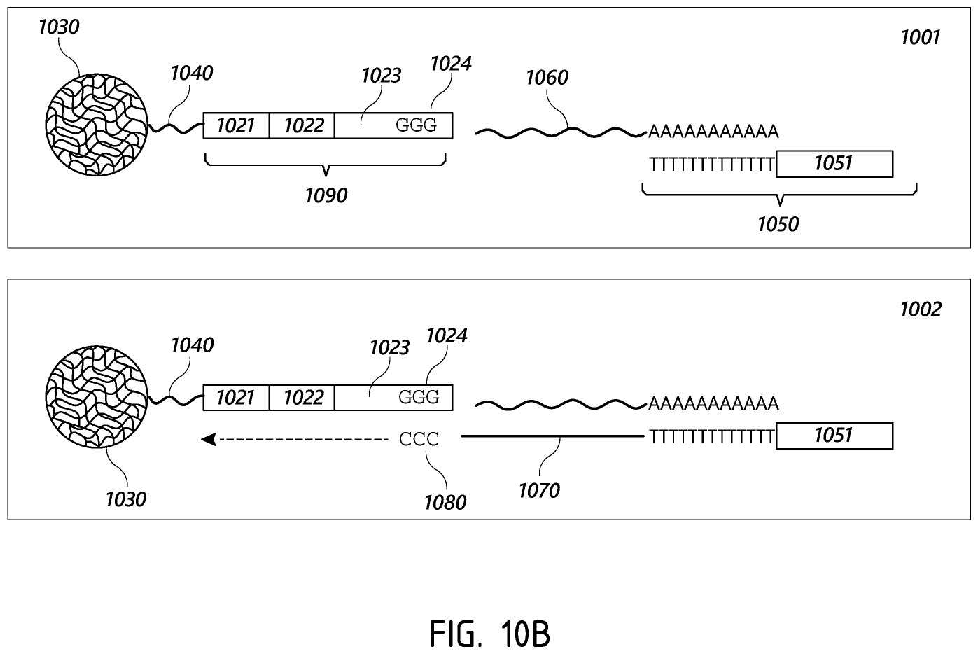

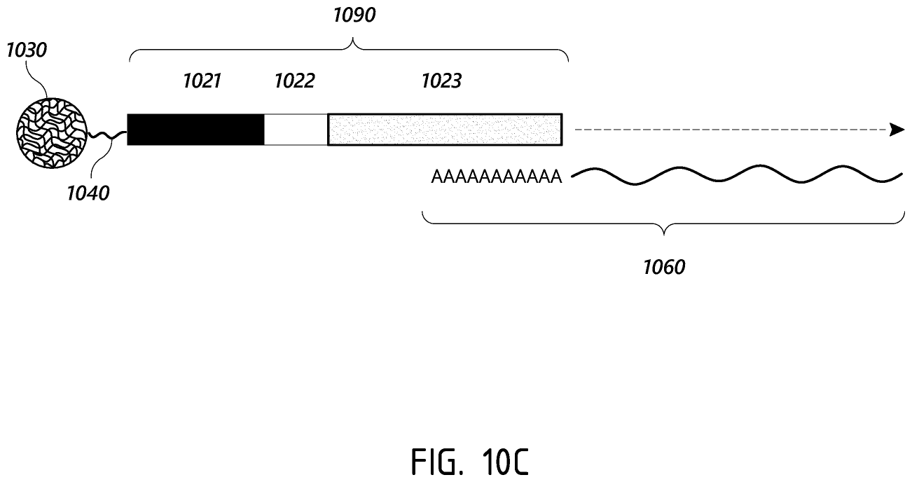

Exemplary barcode molecules attached to a support (e.g., a bead) is shown in FIG. 9. In some embodiments, analysis of multiple analytes (e.g., RNA and one or more analytes using labelling agents described herein) can include nucleic acid barcode molecules as generally depicted in FIG. 9. In some embodiments, nucleic acid barcode molecules 910 and 920 are attached to support 930 via a releasable linkage 940 (e.g., including a labile bond) as described elsewhere herein. Nucleic acid barcode molecule 910 can include functional sequence 911, barcode sequence 912 and capture sequence 913. Nucleic acid barcode molecule 920 can include adapter sequence 921, barcode sequence 912, and capture sequence 923, wherein capture sequence 923 includes a different sequence than capture sequence 913. In some instances, adapter 911 and adapter 921 include the same sequence. In some instances, adapter 911 and adapter 921 include different sequences. Although support 930 is shown including nucleic acid barcode molecules 910 and 920, any suitable number of barcode molecules including common barcode sequence 912 are contemplated herein. For example, in some embodiments, support 930 further includes nucleic acid barcode molecule 950. Nucleic acid barcode molecule 950 can include adapter sequence 951, barcode sequence 912 and capture sequence 953, wherein capture sequence 953 includes a different sequence than capture sequence 913 and 923. In some instances, nucleic acid barcode molecules (e.g., 910, 920, 950) include one or more additional functional sequences, such as a UMI or other sequences described herein. The nucleic acid barcode molecules 910, 920 or 950 can interact with analytes as described elsewhere herein, for example, as depicted in FIGS. 10A-10C.

Referring to FIG. 10A, in an instance where cells are labelled with labelling agents, capture sequence 1023 can be complementary to an adapter sequence of a reporter oligonucleotide. Cells can be contacted with one or more reporter oligonucleotide 1020 conjugated labelling agents 1010 (e.g., polypeptide such as an antigen or fragment of an antigen, antibody, or others described elsewhere herein). In some cases, the cells can be further processed prior to barcoding. For example, such processing steps can include one or more washing and/or cell sorting steps. In some instances, a cell that is bound to labelling agent 1010 which is conjugated to oligonucleotide 1020 and support 1030 (e.g., a bead, such as a gel bead) including nucleic acid barcode molecule 1090 is partitioned into a partition amongst a plurality of partitions (e.g., a droplet of a droplet emulsion or a well of a microwell array). In some instances, the partition includes at most a single cell bound to labelling agent 1010. In some instances, reporter oligonucleotide 1020 conjugated to labelling agent 1010 (e.g., polypeptide such as a target antigen or fragment of a target antigen, an antibody, pMHC molecule such as an MHC multimer, etc.) includes a first functional sequence 1011 (e.g., a primer sequence), a barcode sequence 1012 that identifies the labelling agent 1010 (e.g., the polypeptide such as a target antigen or fragment of a target antigen, antibody, or peptide of a pMHC molecule or complex), and a capture handle sequence 1013. Capture handle sequence 1013 can be configured to hybridize to a complementary sequence, such as capture sequence 1023 present on a nucleic acid barcode molecule 1090 (e.g., partition-specific barcode molecule). In some instances, oligonucleotide 1010 includes one or more additional functional sequences, such as those described elsewhere herein.

Barcoded nucleic acid molecules can be generated (e.g., via a nucleic acid reaction, such as nucleic acid extension, reverse transcription, or ligation) from the constructs described in FIGS. 10A-10C. For example, capture handle sequence 1013 can then be hybridized to complementary capture sequence 1023 to generate (e.g., via a nucleic acid reaction, such as nucleic acid extension or ligation) a barcoded nucleic acid molecule including cell barcode (e.g., common barcode or partition-specific barcode) sequence 1022 (or a reverse complement thereof) and reporter barcode sequence 1012 (or a reverse complement thereof). In some embodiments, the nucleic acid barcode molecule 1090 (e.g., partition-specific barcode molecule) further includes a UMI. Barcoded nucleic acid molecules can then be optionally processed as described elsewhere herein, e.g., to amplify the molecules and/or append sequencing platform specific sequences to the fragments. See, e.g., U.S. Pat. Pub. 2018/0105808. Barcoded nucleic acid molecules, or derivatives generated therefrom, can then be sequenced on a suitable sequencing platform.

In some instances, analysis of multiple analytes (e.g., nucleic acids and one or more analytes using labelling agents described herein) can be performed. For example, the workflow can include a workflow as generally depicted in any of FIGS. 10A-10C, or a combination of workflows for an individual analyte, as described elsewhere herein. For example, by using a combination of the workflows as generally depicted in FIGS. 10A-10C, multiple analytes can be analyzed.

In some instances, analysis of an analyte (e.g. a nucleic acid, a polypeptide, a carbohydrate, a lipid, etc.) includes a workflow as generally depicted in FIG. 10A. A nucleic acid barcode molecule 1090 can be co-partitioned with the one or more analytes. In some instances, nucleic acid barcode molecule 1090 is attached to a support 1030 (e.g., a bead, such as a gel bead), such as those described elsewhere herein. For example, nucleic acid barcode molecule 1090 can be attached to support 1030 via a releasable linkage 1040 (e.g., including a labile bond), such as those described elsewhere herein. Nucleic acid barcode molecule 1090 can include a functional sequence 1021 and optionally include other additional sequences, for example, a barcode sequence 1022 (e.g., common barcode, partition-specific barcode, or other functional sequences described elsewhere herein), and/or a UMI sequence 1025. The nucleic acid barcode molecule 1090 can include a capture sequence 1023 that can be complementary to another nucleic acid sequence, such that it can hybridize to a particular sequence.

For example, capture sequence 1023 can include a poly-T sequence and can be used to hybridize to mRNA. Referring to FIG. 10C, in some embodiments, nucleic acid barcode molecule 1090 includes capture sequence 1023 complementary to a sequence of RNA molecule 1060 from a cell. In some instances, capture sequence 1023 includes a sequence specific for an RNA molecule. Capture sequence 1023 can include a known or targeted sequence or a random sequence. In some instances, a nucleic acid extension reaction can be performed, thereby generating a barcoded nucleic acid product including capture sequence 1023, the functional sequence 1021, UMI sequence 1025, any other functional sequence, and a sequence corresponding to the RNA molecule 1060.

In another example, capture sequence 1023 can be complementary to an overhang sequence or an adapter sequence that has been appended to an analyte. For example, referring to FIG. 10B, in some embodiments, primer 1050 includes a sequence complementary to a sequence of nucleic acid molecule 1060 (such as an RNA encoding for a BCR sequence) from a biological particle. In some instances, primer 1050 includes one or more sequences 101 that are not complementary to RNA molecule 1060. Sequence 1051 can be a functional sequence as described elsewhere herein, for example, an adapter sequence, a sequencing primer sequence, or a sequence the facilitates coupling to a flow cell of a sequencer. In some instances, primer 1050 includes a poly-T sequence. In some instances, primer 1050 includes a sequence complementary to a target sequence in an RNA molecule. In some instances, primer 1050 includes a sequence complementary to a region of an immune molecule, such as the constant region of a BCR sequence. Primer 1050 is hybridized to nucleic acid molecule 1060 and complementary molecule 1070 is generated. For example, complementary molecule 1070 can be cDNA generated in a reverse transcription reaction. In some instances, an additional sequence can be appended to complementary molecule 1070. For example, the reverse transcriptase enzyme can be selected such that several non-templated bases 1080 (e.g., a poly-C sequence) are appended to the cDNA. In another example, a terminal transferase can also be used to append the additional sequence. Nucleic acid barcode molecule 1090 includes a sequence 1024 complementary to the non-templated bases, and the reverse transcriptase performs a template switching reaction onto nucleic acid barcode molecule 1090 to generate a barcoded nucleic acid molecule including cell (e.g., partition specific) barcode sequence 1022 (or a reverse complement thereof) and a sequence of complementary molecule 1070 (or a portion thereof). In some instances, capture sequence 1023 includes a sequence complementary to a region of an immune molecule, such as the constant region of a BCR sequence. Capture sequence 1023 is hybridized to nucleic acid molecule 1060 and a complementary molecule 1070 is generated. For example, complementary molecule 1070 can be generated in a reverse transcription reaction generating a barcoded nucleic acid molecule including cell barcode (e.g., common barcode or partition-specific barcode) sequence 1022 (or a reverse complement thereof) and a sequence of complementary molecule 1070 (or a portion thereof). Additional methods and compositions suitable for barcoding cDNA generated from mRNA transcripts including those encoding V(D)J regions of an immune cell receptor and/or barcoding methods and composition including a template switch oligonucleotide are described in International Patent Application WO2018/075693, U.S. Patent Publication No. 2018/0105808, U.S. Patent Publication No. 2015/0376609, filed Jun. 26, 2015, and U.S. Patent Publication No. 2019/0367969.

Combinatorial Barcoding

In some instances, barcoding of a nucleic acid molecule may be done using a combinatorial approach. In such instances, one or more nucleic acid molecules (which may be comprised in biological particle, e.g., a cell, e.g., a fixed cell, organelle, nucleus, or cell bead) may be partitioned (e.g., in a first set of partitions, e.g., wells or droplets) with one or more first nucleic acid barcode molecules (optionally coupled to a bead). The first nucleic acid barcode molecules or derivative thereof (e.g., complement, reverse complement) may then be attached to the one or more nucleic acid molecules, thereby generating barcoded nucleic acid molecules, e.g., using the processes described herein. The first nucleic acid barcode molecules may be partitioned to the first set of partitions such that a nucleic acid barcode molecule, of the first nucleic acid barcode molecules, that is in a partition comprises a barcode sequence that is unique to the partition among the first set of partitions. Each partition may comprise a unique barcode sequence. For example, a set of first nucleic acid barcode molecules partitioned to a first partition in the first set of partitions may each comprise a common barcode sequence that is unique to the first partition among the first set of partitions, and a second set of first nucleic acid barcode molecules partitioned to a second partition in the first set of partitions may each comprise another common barcode sequence that is unique to the second partition among the first set of partitions. Such barcode sequence (unique to the partition) may be useful in determining the cell or partition from which the one or more nucleic acid molecules (or derivatives thereof) originated.

The barcoded nucleic acid molecules from multiple partitions of the first set of partitions may be pooled and re-partitioned (e.g., in a second set of partitions, e.g., one or more wells or droplets) with one or more second nucleic acid barcode molecules. The second nucleic acid barcode molecules or derivative thereof may then be attached to the barcoded nucleic acid molecules. As with the first nucleic acid barcode molecules during the first round of partitioning, the second nucleic acid barcode molecules may be partitioned to the second set of partitions such that a nucleic acid barcode molecule, of the second nucleic acid barcode molecules, that is in a partition comprises a barcode sequence that is unique to the partition among the second set of partitions. Such barcode sequence may also be useful in determining the cell or partition from which the one or more nucleic acid molecules or first barcoded nucleic acid molecules originated. The barcoded nucleic acid molecules may thus comprise two barcode sequences (e.g., from the first nucleic acid barcode molecules and the second nucleic acid barcode molecules).

Additional barcode sequences may be attached to the barcoded nucleic acid molecules by repeating the processes any number of times (e.g., in a split-and-pool approach), thereby combinatorically synthesizing unique barcode sequences to barcode the one or more nucleic acid molecules. For example, combinatorial barcoding may comprise at least 1, 2, 3, 4, 5, 6, 7, 8, 9, 10 or more operations of splitting (e.g., partitioning) and/or pooling (e.g., from the partitions). Additional examples of combinatorial barcoding may also be found in International Patent Publication Nos. WO2019/165318, each of which is herein entirely incorporated by reference for all purposes.

Beneficially, the combinatorial barcode approach may be useful for generating greater barcode diversity, and synthesizing unique barcode sequences on nucleic acid molecules derived from a cell or partition. For example, combinatorial barcoding comprising three operations, each with 100 partitions, may yield up to 106 unique barcode combinations. In some instances, the combinatorial barcode approach may be helpful in determining whether a partition contained only one cell or more than one cell. For instance, the sequences of the first nucleic acid barcode molecule and the second nucleic acid barcode molecule may be used to determine whether a partition comprised more than one cell. For instance, if two nucleic acid molecules comprise different first barcode sequences but the same second barcode sequences, it may be inferred that the second set of partitions comprised two or more cells.

In some instances, combinatorial barcoding may be achieved in the same compartment. For instance, a unique nucleic acid molecule comprising one or more nucleic acid bases may be attached to a nucleic acid molecule (e.g., a sample or target nucleic acid molecule) in successive operations within a partition (e.g., droplet or well) to generate a barcoded nucleic acid molecule. A second unique nucleic acid molecule comprising one or more nucleic acid bases may be attached to the barcoded nucleic acid molecule. In some instances, all the reagents for barcoding and generating combinatorially barcoded molecules may be provided in a single reaction mixture, or the reagents may be provided sequentially.

In some instances, cell beads comprising nucleic acid molecules may be barcoded. Methods and systems for barcoding cell beads are further described in PCT/US2018/067356 and U.S. Pat. Pub. No. 2019/0330694, which are hereby incorporated by reference in its entirety

### Further Methods of the Disclosure

In certain methods of the disclosure relating to new approaches and methods of: (i) engineering an antigen-binding site of an antibody, or antigen-binding fragment thereof, or (ii) preparing a library of variant antibodies, or variant antigen-binding fragments thereof, that include an altered characteristic(s), steps that include embodiments and elements related to paritions, partitioning, reporter oligonucleotides and generation of barcoded nucleic acid molecules are not required.

In such methods, as in all embodiments of the methods described herein, a nucleic acid sequence encoding a selected antibody, or selected antigen-binding fragment thereof, is provided. In these particular embodiments, the provided nucleic acid sequence encodes a selected antibody derived from nucleic acids of a cell of a human donor, or a selected antigen-binding fragment thereof. Because the provided nucleic acid sequence encoding the selected antibody, or selected antigen-binding fragment thereof, is derived from nucleic acids of a cell of a human donor, any variant of the selected antibody (or selected antigen-binding fragment thereof) identified by these particular embodiments of the methods will be derisked for immunogenicity, e.g., and will have more predictable pharmacokinetics, in a recipient when employed in a therapeutic setting.

The selected antibody derived from nucleic acids of the cell of the human donor, or selected antigen-binding fragment thereof, may be a full human antibody molecule, or a human immunoglobulin molecule including four polypeptide chains, two heavy chains (HCs) and two light chains (LCs) inter-connected by disulfide bonds, or a multimer thereof (e.g. IgM). The selected antibody derived from nucleic acids of the cell of the human donor may be of any isotype, having any, Immunoglobulin (Ig)A (e.g., IgA1 or IgA2), IgD, IgE, IgG (e.g., IgG1, IgG2, IgG3 and IgG4) or IgM constant region.

The selected antibody derived from nucleic acids of the cell of the human donor, or selected antigen-binding fragment thereof, may be a fragment of the selected antibody derived from nucleic acids of the cell of the human donor. As a fragment of the selected antibody of the cell of the human donor, it may be any naturally occurring, enzymatically obtainable, synthetic, or genetically engineered fragment from the selected antibody of the cell of the human donor. The fragment of the selected antibody of the cell of the human donor may be any one of: (i) Fab fragments; (ii) F(ab′)2 fragments; (iii) Fd fragments; (iv) Fv fragments; (v) single-chain Fv (scFv) molecules; (vi) sdAb fragments; or (vii) minimal recognition units consisting of the amino acid residues that mimic the hypervariable region of an antibody (e.g., an isolated complementarity determining region (CDR) such as a CDR3 peptide), or a constrained FWR3-CDR3-FWR4 peptide. Further, it may be an engineered molecule, such as a domain-specific antibody, single domain antibody, diabody, triabody, tetrabody, minibody, nanobody (e.g., monovalent nanobodies, bivalent nanobodies, etc.) or a small modular immunopharmaceutical (SMIP).

The selected antibody derived from nucleic acids of the cell of the human donor, or selected antigen-binding fragment thereof, may be selected due to its ability to bind a target antigen. The target antigen to which the selected antibody, or antigen-binding fragment thereof, may bind may be any antigen for which the development and identification of variants of the selected antibody, or selected antigen-binding fragment thereof, is desirable. Target antigens for all embodiments of the methods provided herein have earlier been described. Broadly, target antigens may be an antigen associated with a pathogen or infectious agent, such as a viral, bacterial, parasitic, protozoal or prion agent, or target antigens may be associated with tumors, cancers, inflammatory or autoimmune diseases or disorders.

Further, and also as described as applicable to all embodiments of the methods provided herein, the selected antibody derived from nucleic acids of the cell of the human donor, or selected antigen-binding fragment thereof may be selected (as are other selected antibodies, or selected antigen-binding fragments thereof) due to their ability to bind target antigens at regions of interest, e.g., at one or more epitopes or domains of the target antigen that are involved in a signaling pathway, that interact with other proteins or peptides, or that result in or prevent a conformational change in the target antigen.

The provided nucleic acid sequence encoding the selected antibody derived from nucleic acids of the cell of the human donor, or selected antigen-binding fragment thereof, may be provided as a nucleic acid sequence of any type, e.g., DNA/cDNA molecule, suitable for amplification. The provided nucleic acid sequence encoding the selected antibody, or selected antigen-binding fragment thereof, may be provided as a plurality, or library, of nucleic acid sequences, e.g., cDNA molecules. The provided nucleic acid sequence encoding the selected antibody, or selected antigen-binding fragment thereof, may be provided as a nucleic acid sequence, e.g., cDNA, molecule cloned into a vector. The provided nucleic acid sequence encoding the selected antibody, or selected antigen-binding fragment thereof, may be provided as a plurality of nucleic acid sequences, e.g., cDNA molecules, cloned into a plurality of vectors. The provided nucleic acid sequence encoding the selected antibody, or selected antigen-binding fragment thereof, may be configured in such a way as to facilitate amplification by any method, e.g., including rolling circle amplification.

The provided nucleic acid sequence encoding the selected antibody derived from nucleic acids of the cell of the human donor, or selected antigen-binding fragment thereof, may have been derived from a cell of the B cell lineage, or of a sample of cells that includes B cells, e.g., memory B cells, obtained from a human donor. The cell or sample of cells, may be a cell of, or sample of cells from, the human donor obtained from a blood sample, a peripheral blood mononuclear cell sample or a plasma sample.

The provided nucleic acid sequence encoding the selected antibody derived from nucleic acids of the cell of the human donor, or selected antigen-binding fragment thereof, need not have been derived from a single cell or a single cell sample of a single donor. It may have been derived from having combined cells, or samples of cells, of multiple donors, e.g., cells or cell samples of more than one human. If the cells or the samples of cells are of multiple donors, the cells or samples of cells may be any combination of one or more of human blood samples, human peripheral blood mononuclear cell samples or human plasma samples.

The human donor whose cells the nucleic acid sequence encoding the selected antibody or selected antigen-binding fragment thereof, is derived may be known to have been exposed to the target antigen. Alternatively, the human donor may suspected of having been exposed to the target antigen. The human donor may be known or expected to be resistant to a pathogen or infectious agent that bears the target antigen.

The cell or cells of the human donor, may have been subject to certain processing steps prior to providing the nucleic acid sequence encoding selected antibody, or selected antigen-binding fragment thereof, that: (i) identified the cell of the human donor as expressing the selected antibody, or selected antigen-binding fragment thereof, and (ii) prepared the nucleic acid sequences encoding the selected antibody, or antigen-binding fragment thereof, for the providing. If such prior processing steps were performed, prior to providing the nucleic acid sequence encoding selected antibody, or selected antigen-binding fragment thereof, they may have included contacting the cell of the human donor with the target antigen, wherein the target antigen was coupled to a reporter oligonucleotide. The contacting of the cell with the target antigen may have involved providing a reaction mixture comprising the cell and the target antigen. The cell of the human donor bound to the target antigen may have been partitioned in a partition that further included a plurality of nucleic acid barcode molecules, wherein the plurality of nucleic acid barcode molecules included a partition-specific barcode sequence. In the partition, barcoded nucleic acid molecules may have been generated. The generated barcoded nucleic acid molecules may have included first and second barcoded nucleic acid molecules. The first generated barcoded nucleic acid molecule may have included the sequence of the reporter oligonucleotide or a reverse complement thereof and the partition-specific barcode sequence or reverse complement thereof; the detection of the first barcoded nucleic acid molecule may have identified the cell of the human donor as expressing the selected antibody, or the selected antigen-binding fragment thereof. The generated second barcoded nucleic acid molecule may have included the nucleic acid sequence encoding the selected antibody, or antigen-binding fragment thereof, for the providing. Extensive detail with respect to elements and steps that may be included in, or as part of, methods in which cells are incubated with target antigen (coupled to a reporter oligonucleotide), cells coupled to target antigen are partitioned into a partition (further including a plurality of nucleic acid barcode molecules comprising a partition-specific barcode sequence), and generating barcoded nucleic acid molecules in partition, have been provided elsewhere in the disclosure herein. It will also be understood that in any of the methods provided herein, e.g., methods in which a nucleic acid sequence encoding a selected antibody, or selected antigen-binding fragment thereof, is provided from a cell(s) or a sample(s) of cell(s), these certain processing steps may be, or have been, performed.

The nucleic acid sequence encoding the selected antibody, or selected antigen-binding fragment thereof, derived from nucleic acids of the cell of the human donor may be amplified via error-prone amplification. Error-prone amplification, conditions for performing error-prone amplification and alterations resulting having performed error-prone amplification have been earlier-discussed for all embodiments of the methods provided herein. The error-prone amplification, as also discussed for all embodiments of the methods herein, produce variant antibodies, or variant antigen-binding fragments thereof.

The variant antibodies, or variant antigen binding fragments thereof, are expressed in a plurality of mammalian cells, and a mammalian cell of the plurality of mammalian cells may express a variant antibody (or variant antigen-binding fragment thereof) of the plurality of variant antibodies (or plurality of variant antigen-binding fragments thereof). The mammalian cells that express the variant antibodies, or variant antigen-binding fragments thereof, may be BHK cells, CHO cells, Vero cells, human A549 cells, human cervix cells, human CHME5 cells, PER.C6 cells, NS0 cells, human epidermoid larynx cells, human fibroblast cells, HEK-293 cells, HeLa cells, HepG2 cells, HUH-7 cells, MRC-5 cells, human muscle cells, 3T3 cells, mouse connective tissue cells, mouse muscle cells or rabbit kidney cells. These cells, and others suitable for expression of a variant antibody, or variant antigen-binding fragment thereof, are available from many sources, including the American Type Culture Collection (Manassas, VA). An advantage of expressing the variant antibody, or variant antigen-binding fragment thereof, in a mammalian cell is that glycoslation of the variant antibody, or variant antigen-binding fragment thereof, will more closely resemble that of a human antibody. By being glycosylated like a human antibody, the variant antibody, or variant antigen-binding fragment thereof, will have a structure most relevant to its use as a human therapeutic for analysis in assays, e.g., activity assays, and use as a therapeutic. In some embodiments of these methods, the variant antibody, or variant antigen-binding fragment thereof, will not have been expressed in a non-mammalian cell, e.g., phage, bacteria or insect cell, between the steps of amplifying via error-prone amplification and expressing the plurality of variant antibodies, or variant antigen-binding fragments thereof, in a plurality of mammalian cells.

Suitable vectors, and methods, for introduction of the nucleic acid sequence encoding the variant antibody, or variant antigen-binding fragment thereof, are known in the art and are commercially available, or readily prepared by a skilled artisan. Many of these have been discussed herein for all embodiments of the methods of the disclosure, and can be further found in, for example, Sambrook, J., & Russell, D. W. (2012). Molecular Cloning: A Laboratory Manual (4th ed.). Cold Spring Harbor, NY: Cold Spring Harbor Laboratory and Sambrook, J., & Russel, D. W. (2001). Molecular Cloning: A Laboratory Manual (3rd ed.). Cold Spring Harbor, NY: Cold Spring Harbor Laboratory (jointly referred to herein as “Sambrook”); Ausubel, F. M. (1987). Current Protocols in Molecular Biology. New York, NY: Wiley (including supplements through 2014); Bollag, D. M. et al. (1996). Protein Methods. New York, NY: Wiley-Liss; Huang, L. et al. (2005). Nonviral Vectors for Gene Therapy. San Diego: Academic Press; Kaplitt, M. G. et al. (1995). Viral Vectors: Gene Therapy and Neuroscience Applications. San Diego, CA: Academic Press; Lefkovits, I. (1997). The Immunology Methods Manual: The Comprehensive Sourcebook of Techniques. San Diego, CA: Academic Press; Doyle, A. et al. (1998). Cell and Tissue Culture: Laboratory Procedures in Biotechnology. New York, NY: Wiley; Mullis, K. B., Ferre, F. & Gibbs, R. (1994). PCR: The Polymerase Chain Reaction. Boston: Birkhauser Publisher; Greenfield, E. A. (2014). Antibodies: A Laboratory Manual (2nd ed.). New York, NY: Cold Spring Harbor Laboratory Press; Beaucage, S. L. et al. (2000). Current Protocols in Nucleic Acid Chemistry. New York, NY: Wiley, (including supplements through 2014); and Makrides, S. C. (2003). Gene Transfer and Expression in Mammalian Cells. Amsterdam, NL: Elsevier Sciences B.V., the disclosures of which are incorporated herein by reference).

From the expression of the variant antibody, or variant antigen-binding fragment thereof, it may be identified as engineered to have the altered characteristic. From the expression of the variant antibody, or variant antigen-binding fragment thereof, it may be identified for inclusion in the library. The altered characteristic may be altered binding affinity. The altered binding affinity may be a higher affinity, such as a 5% higher, 10% higher, 15% higher, 20% higher, 25% higher, 30% higher, 35% higher, 40% higher, 45% higher 50% higher, 55% higher, 60% higher, 65% higher, 70% higher, 75% higher, 80% higher, 85% higher, 90% higher, 95% higher, 100% higher, 150% higher, 200% higher, 250% higher, 300% higher, 350% higher, 400% higher, 450% higher, 500% higher relative to the binding affinity of the selected antibody, or antigen-binding fragment thereof, for the target antigen. The altered binding affinity may be 10×, 100× or 1000× higher relative to the binding affinity of the selected antibody, or antigen-binding fragment thereof, for the target antigen. The altered binding affinity may be lower binding affinity. The lower binding affinity may be 1%, lower, 2% lower, 3% lower, 4% lower, 5% lower, 10% lower, 15% lower or 20% lower. It may be at most 1% lower, at most 2% lower, at most 3% lower, at most 4% lower, at most 5% lower, at most 10% lower, at most 15% lower or at most 20% lower than the binding affinity of the selected antibody, or antigen-binding fragment thereof, for the target antigen. The altered binding affinity may be 100% lower than the binding affinity of the selected antibody, or antigen-binding fragment thereof, for the target antigen. Binding affinity may be determined by any of a number techniques known in the art including, for example, competition binning and competition enzyme-linked immunosorbent assay (ELISA), NMR or HDX-MS, Surface Plasmon Resonance (SPR), e.g. by using a Biacore™ system, or KinExA.

The altered characteristic that identifies the variant antibody, or variant antigen-binding fragment thereof, or that may result in inclusion of the variant antibody, or variant antigen-binding fragment thereof, as being in the library may be one or more of: an altered association constant, an altered dissociation constant, or altered specificity to the target antigen, e.g., alteration from multispecificity that includes the target antigen to more selective specificity or specificity for only binding to the target antigen. Alternatively, or in addition, the altered characteristic may be an altered activity. The altered activity may be altered neutralization, if the selected antibody, or selected antigen-binding fragment thereof, is selected due to its binding a target antigen that is associated with a pathogen, e.g., a virus. An altered activity may be altered autoimmune activity, if the selected antibody, or selected antigen-binding fragment thereof, is selected due to its binding a target antigen that is associated with inflammation, e.g., cytokine or cytokine receptor. An altered activity may be altered anti-tumor activity, if the selected antibody or selected antigen-binding fragment thereof, is selected due to its binding a target antigen that is associated with a growth factor or growth factor receptor implicated in cancer, e.g., EGFR or IGFR.

As in other embodiments of the methods provided herein, sequences of variant antibodies, or variant antigen-binding fragments thereof, may be determined. The sequences may be determined by any suitable method, including those described elsewhere herein.

### Enrichment

In embodiments of the methods provided herein, a nucleic acid sequence encoding a selected antibody, or selected antigen-binding fragment thereof, is provided. In particular embodiments of the methods provided herein, the provided nucleic acid sequence may be derived from a cell, cells, a sample or samples of a donor, e.g., a human donor. In particular embodiments of the methods provided herein, the provided nucleic acid sequence may be derived from a barcoded nucleic acid molecule, e.g., a second barcoded nucleic acid molecule described herein, that was derived from a cell or nucleus thereof, cells or nuclei thereof, a sample or samples of a donor, e.g., a human donor. To derive a nucleic acid sequence encoding an antibody from a cell(s) or sample(s) of one or more donors, e.g., human donors, it may be useful to process the nucleic acid molecules of the cell(s) or cells of the sample(s), or barcoded nucleic acid molecules from the cell(s), cells of the sample(s), or nuclei thereof, by steps that include enriching for the nucleic acid molecules that encode the selected antibody or selected antigen-binding fragment thereof.

Additionally, in embodiments of the methods provided herein, the sequences encoding variant antibodies, or variant antigen-binding molecules thereof, may be determined from cells expressing the variant antibodies, or variant antigen-binding fragments thereof. In particular embodiments of the methods provided herein, the sequences encoding variant antibodies, or variant antigen-binding molecules thereof may be determined from barcoded nucleic acid molecules derived from cells expressing the variant antibodies. To sequence nucleic acid molecules encoding the variant antibodies, or variant antigen-binding molecules thereof, from the cells expressing them, it may be useful to process the nucleic acid molecules of the cells by steps including steps that enrich for those nucleic acid molecules that encode the variant antibodies or variant antigen-binding fragments thereof. In some embodiments, the enrichment may be performed according to methods described in PCT/US2017/057269, which is hereby incorporated by reference in its entirety.

Enrichment for nucleic acid molecules encoding antibodies, or antigen-binding fragments thereof (regardless of whether the antibody, or antigen-binding fragment thereof, is “selected” or “variant”) from nucleic acid sequence of a cell(s), or cell(s) of a sample(s), may be performed according to a process that includes one or more amplification reactions where the one or more amplification reactions employ a primer or primers with nucleic acid sequences configured to be complementary to regions of nucleic acid molecules comprising sequences encoding the antibodies, or antigen-binding fragments thereof, in the cell(s), or the cell(s) of the sample(s) and that will selectively amplify them.

In some embodiments, the enrichment is carried out using an error-prone amplification reaction disclosed herein to produce polynucleotides encoding variant antibodies or variant antigen-binding fragments thereof.

Enrichment, by one or more such amplification reactions, e.g., an error prone amplification reaction described herein, may be performed with forward and reverse primers (which may also be referred to as first and second primers, respectively). A forward primer, for use in such an amplification reaction, may configured to be complementary to nucleic acid sequences at the 5′ untranslated region, or at the 5′ coding sequence, of nucleic acid molecules encoding the antibody or antigen-binding fragment thereof, e.g., sequence of a leader for, or variable region of, an antibody (or antigen-binding fragment thereof) coding sequence. Enrichment, by one or more such amplification reactions, e.g., an error prone amplification reaction described herein, may be performed with a forward primer configured to be complementary to one or more functional sequences (e.g., barcode sequences, primer binding sequences, TSO sequences, or UMI sequences, as described earlier herein) attached to the nucleic acid sequence encoding the antibody, or antigen-binding fragment thereof, in an earlier processing step as described herein. If functional sequences have been attached to the nucleotide sequence encoding the antibody, or antigen binding fragment thereof, during an earlier processing step, then the forward primer may be configured to be complementary to one or more of the functional sequences, e.g., one or more of a barcode, UMI, TSO or read sequence, or portions thereof. In some embodiments, the forward primer is configured to be complementary to one or more of a barcode sequence or a portion thereof, e.g., a partition-specific barcode sequence or portion thereof, a UMI sequence or a portion thereof, and a read sequence or a portion thereof. In some embodiments, the forward primer is configured to be complementary to one or more of a barcode sequence, e.g., a partition-specific barcode sequence or portion thereof and a UMI sequence or portion thereof. In some embodiments, the forward primer is configured to be complementary to a barcode sequence, e.g., a partition-specific barcode sequence or portion thereof. In some embodiments, the forward primer is configured to be complementary to a UMI sequence or portion thereof.

Enrichment, by one or more such amplification reactions, e.g., an error prone amplification reaction described herein, and performed with any of these forward primers, may also be performed with a reverse primer configured to be complementary to a nucleic acid sequence: (i) at the 3′ end of the variable sequence of the antibody, or antigen-binding fragment thereof, (ii) encoding a portion of any of a V region, a D region, or a J region of the antibody, or antigen-binding fragment thereof, (iii) encoding a portion of the constant region of the antibody, or antigen-binding fragment thereof, (iv) encoding framework 4 of the antibody, or antigen-binding fragment thereof, (v) encoding a junction (J) region and/or isotype region of the antibody, or fragment thereof, or (vi) encoding a complementarity region (CDR)3 of the antibody, or antigen-binding fragment thereof, or (vii) a junction between any one or more the J region, D region, and/or V region, of the BCR or fragment thereof (or a complement of any of (i)-(vii) thereof).

Enrichment, by the one or more amplification reactions, e.g., an error prone amplification reaction described herein, may be performed with a forward and reverse primer pair selected from: (i) a forward primer configured to be complementary to at least a portion of a cell barcode and UMI sequence that had been attached to the nucleic acid sequence encoding the antibody, or antigen-binding fragment thereof, during an earlier processing step and a reverse primer configured to be complementary to a nucleic acid sequence encoding at least a portion of the isotype or constant region of the antibody, or antigen-binding fragment thereof (or a complement thereof); (ii) a forward primer configured to be complementary to a portion of a leader for the coding sequence of the antibody, or antigen-binding fragment thereof and/or a portion the coding sequence of the framework 1 of the antibody, or antigen-binding fragment thereof, and a reverse primer configured to be complementary to nucleic acid sequences encoding a portion of CDR3, FWR4, a J region, a D region, and/or a V region, or a junction between any one or more thereof, of the antibody, or antigen-binding fragment thereof (or a complement thereof); (iii) a forward primer configured to be complementary to portions of the cell barcode and UMI sequences that had been attached to the nucleic acid sequence encoding the antibody, or antigen-binding fragment thereof, during an earlier processing step and a reverse primer configured to be complementary to a nucleic acid sequence encoding at least a portion of the isotype and the J regions of the antibody, or antigen-binding fragment thereof (or a complement thereof); or (iv) a forward primer configured to be complementary to a portion of the leader for the coding sequence of the antibody, or antigen-binding fragment thereof, and/or framework 1 of the coding sequence of the antibody, or antigen-binding fragment thereof, a reverse primer configured to be complementary to nucleic acid sequences encoding at least portions of the CDR3, and junction extending into the J region of the antibody or fragment thereof (or a complement thereof).

Enrichment (e.g., via an error prone amplification reaction described herein) for the nucleic acid molecules encoding antibodies, or antigen-binding fragments thereof (regardless of whether the antibody, or antigen-binding fragment thereof, is “selected” or “variant”) from nucleic acid sequence of a cell(s), or cell(s) of a sample(s), may be performed via first and second, e.g., nested, amplification reactions using first and second sets of forward and reverse primers. If nested amplification reactions are performed, the first and second amplification reactions may be performed using: (i) a first forward and reverse primer set comprising a first forward primer configured to be complementary to at least portions of cell barcode and UMI sequences that had been attached to the nucleic acid sequence encoding the antibody, or antigen-binding fragment thereof, during an earlier processing step and a first reverse primer configured to be complementary to at least a portion of the coding sequence of the constant region of the antibody (or a complement thereof), and a second forward and reverse primer set comprising a second forward primer configured to be complementary to a coding sequence for the V(D)J region of the antibody, or antigen-binding fragment thereof, and a second reverse primer configured to be complementary to at least a portion of the coding sequence downstream of the V(D)J region of the antibody, or antigen-binding fragment thereof e.g., in the constant region (or a complement thereof); (ii) a first forward and reverse primer set comprising a first forward primer configured to be complementary to at least a portion of a barcode attached to the nucleic acid sequence encoding the antibody, or antigen-binding fragment thereof, during a processing step and a first reverse primer configured to be complementary to a read sequence attached to the nucleic acid sequence encoding the antibody, or antigen-binding fragment thereof, during a processing step (or complement thereof), and a second forward and reverse primer set comprising a second forward primer configured to be complementary to a coding sequence for the V region of the antibody, or antigen-binding fragment thereof, and a second reverse primer configured to be complementary to at least a portion of a coding sequence downstream of the constant region and J region of the antibody, or antigen-binding fragment thereof (or a complement thereof); (iii) a first forward and reverse primer set comprising a first forward primer configured to be complementary to portions of a cell barcode and UMI sequence that had been attached to the nucleic acid sequence encoding the antibody, or antigen-binding fragment thereof, during an earlier processing step and a first reverse primer configured to be complementary to at least a portion of the coding sequence for the J region of the antibody, or antigen-binding fragment thereof (or a complement thereof), and a second forward and reverse primer set comprising a second forward primer configured to be complementary to a portion of a leader sequence for the coding sequence of the antibody, or antigen-binding fragment thereof, and a second reverse primer configured to be complementary to a portion of the coding sequence for CDR3 of the antibody, or antigen-binding fragment thereof (or a complement thereof); (iv) a first forward and reverse primer set comprising a first forward primer configured to be complementary to portions of cell barcode and UMI sequences that had been attached to the nucleic acid sequence encoding the antibody, or antigen-binding fragment thereof, during an earlier processing step in combination with a first reverse primer configured to be complementary to a sequence encoding framework 4 of the antibody, or antigen-binding fragment thereof (or a complement thereof), and a second forward and reverse primer set comprising a second forward primer configured to be complementary to a leader sequence to the antibody, or antigen-binding fragment thereof, coding sequence in combination with a second reverse primer configured to be complementary to at least a portion of the coding sequence of the antibody's, or antigen binding fragment thereof's, framework 4 and constant regions (or a complement thereof); or (v) a first forward and reverse primer set comprising a first forward primer configured to be complementary to at least portions of a cell barcode and UMI sequences that had been attached to the nucleic acid sequence encoding the antibody, or antigen-binding fragment thereof, during an earlier processing step in combination with a first reverse primer configured to be complementary to a portion of a sequence encoding at least portions of the isotype and J regions of the antibody, or antigen-binding fragment thereof (or a complement thereof), and a second forward and reverse primer set comprising a second forward primer configured to be complementary to at least a portion of a leader sequence to the antibody, or antigen-binding fragment thereof, coding sequence and/or encoding at least a portion of the sequence encoding framework 1 of the antibody, or antigen-binding fragment thereof, in combination with a second reverse primer configured to be complementary to at least a portion of the coding sequence of the CDR3 and junction extending into the J region of the antibody, or antigen-binding fragment thereof (or a complement thereof).

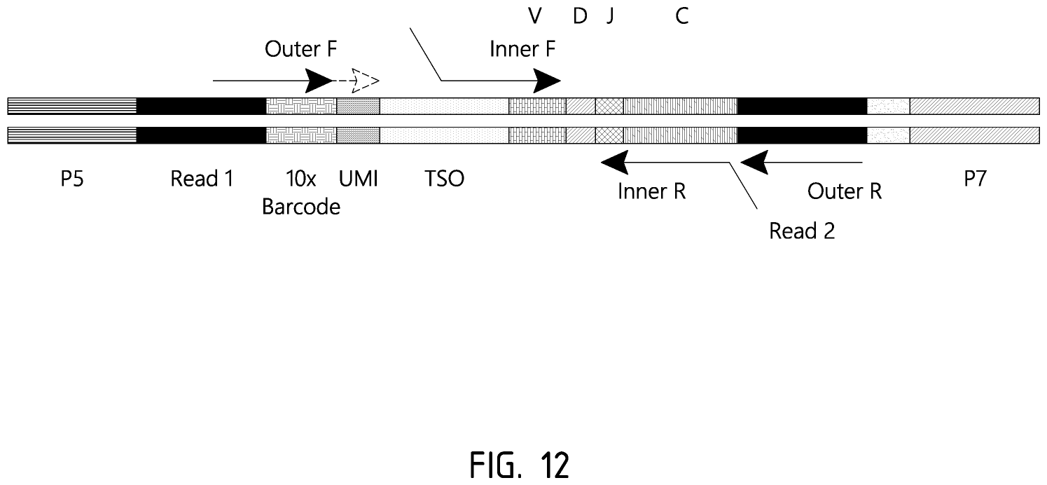

FIG. 12 provides an illustration of an example nested PCR scheme for enriching for the nucleic acid molecules encoding antibodies, or antigen-binding fragments thereof. Such example nested PCR scheme can be employed in an error-prone amplification reaction described herein to produce one or more polynucleotides encoding variant antibodies or variant antigen-binding fragments thereof.

In some embodiments forward and reverse primers of a primer pair may further comprise non-binding handles. A non-binding handle may be a nucleic acid sequence of the forward and/or reverse primer that is not complementary to the nucleic acid molecules comprising sequences encoding antibodies, or antigen-binding fragments thereof (regardless of whether the antibody, or antigen-binding fragment thereof, is “selected” or “variant”). The non-binding handles may be useful for cloning products of the amplification reaction into a recipient vector or to permit pairing of specific heavy and light chain sequences of the nucleic acid molecules encoding the antibodies, or antigen-binding fragments thereof using overlap extension, or a similar method. In embodiments that employ first and second, e.g., nested, amplification reactions using first and second sets of forward and reverse primers, the second set of primers, or inner primers, may include the non-binding handles.

Furthermore, additional detailed disclosure, relating to enrichment for nucleic acid molecules encoding antibodies, or antigen-binding fragments thereof (regardless of whether the antibody, or antigen-binding fragment thereof, is “selected” or “variant”) from nucleic acid sequence of a cell(s), or cell(s) of a sample(s), may be found in U.S. application Ser. No. 63/033,787 filed Jun. 2, 2020 and U.S. application Ser. No. 63/051,257 filed Jul. 13, 2020, which applications are hereby incorporated by reference in their entirety.

### Systems

The present disclosure also provides for systems for engineering an antigen-binding site of an antibody, or antigen-binding fragment thereof, to comprise an improved characteristic. Such systems may include: (i) reagents for performing error-prone amplification, (ii) a target antigen coupled to a reporter oligonucleotide, (iii) a plurality of nucleic acid barcode molecules comprising a partition-specific barcode sequence, and (iv) reagents for generating a plurality barcoded nucleic acid molecules formed by complementary base pairing of (a) a capture sequence of the plurality of nucleic acid barcode molecules and (b) a capture handle sequence of the first reporter oligonucleotide and/or a capture handle sequence of a polynucleotide encoding an engineered variant of the antibody or antigen-binding fragment thereof. The system may further include (iv) reagents for generating a plurality barcoded nucleic acid molecules formed by complementary base pairing of (a) a capture sequence of the plurality of nucleic acid barcode molecules and (b) a capture handle sequence of the first reporter oligonucleotide and/or a capture handle sequence of a polynucleotide encoding an engineered variant of the antibody or antigen-binding fragment thereof. In addition, the system may include a sequencer or sequencing system.

Systems of the disclosure may include reagents for determining affinity of the engineered antibody, or antigen-binding fragment thereof, from a second barcoded nucleic acid molecule of the plurality of barcoded nucleic acid molecules, formed from complementary base pairing of the capture sequence of the plurality of nucleic acid barcode molecules and the capture handle sequence of the first reporter oligonucleotide. The system may further include an analysis engine and/or a network.

As the generation of the plurality of barcoded nucleic acid molecules formed by complementary base pairing of (a) a capture sequence of the plurality of nucleic acid barcode molecules and (b) a capture handle sequence of the first reporter oligonucleotide and/or a capture handle sequence of a polynucleotide encoding an engineered variant of the antibody or antigen-binding fragment thereof may occur in a partition, the systems provided herein may further include a microfluidic device for generating a partition.

Disclosures of embodiments of reagents for use in the system have been described herein. A discussion of systems that operate within the system for engineering an antigen-binding site of an antibody, or antigen-binding fragment thereof, is provided below.

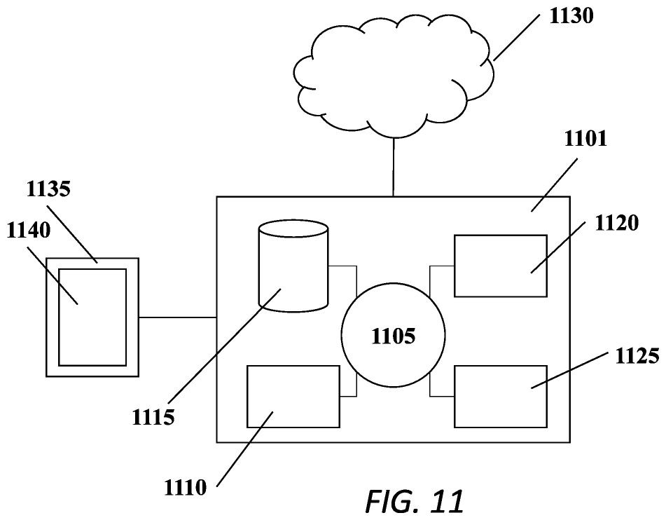

FIG. 11 depicts a block diagram illustrating an example of a system, e.g., computer system, 1101, in accordance with some example system embodiments as provided herein. Referring to FIG. 11, the computer system 1101 may be configured to implement one or more of the analysis engine 1102, the sequencing platform 1104, and the client device 1106. The computer system 1101 can be an electronic device of a user or a computer system that is remotely located with respect to the electronic device. The electronic device can be a mobile electronic device.

The computer system 1101 includes a central processing unit (CPU, also “processor” and “computer processor” herein 1105, which can be a single core or multi core processor, or a plurality of processors for parallel processing. The computer system 1101 also includes memory or memory location 1110 (e.g., random-access memory, read-only memory, flash memory), electronic storage unit 1115 (e.g., hard disk), communication interface 1120 (e.g., network adapter) for communicating with one or more other systems, and peripheral devices 1125, such as cache, other memory, data storage and/or electronic display adapters. The memory 1110, storage unit 1115, interface 1120 and peripheral devices 1125 are in communication with the CPU 1105 through a communication bus (solid lines), such as a motherboard. The storage unit 1115 can be a data storage unit (or data repository) for storing data. The computer system 1101 can be operatively coupled to a computer network (“network”) 1130 with the aid of the communication interface 1120. The network 1130 can be the Internet, an internet and/or extranet, or an intranet and/or extranet that is in communication with the Internet. The network 1130 in some cases is a telecommunication and/or data network. The network 1130 can include one or more computer servers, which can enable distributed computing, such as cloud computing. The network 1130, in some cases with the aid of the computer system 1101, can implement a peer-to-peer network, which may enable devices coupled to the computer system 201 to behave as a client or a server.

The CPU 1105 can execute a sequence of machine-readable instructions, which can be embodied in a program or software. The instructions may be stored in a memory location, such as the memory 1110. The instructions can be directed to the CPU 1105, which can subsequently program or otherwise configure the CPU 1105 to implement methods of the present disclosure. Examples of operations performed by the CPU 1105 can include fetch, decode, execute, and writeback.

The CPU 1105 can be part of a circuit, such as an integrated circuit. One or more other components of the system 1101 can be included in the circuit. In some cases, the circuit is an application specific integrated circuit (ASIC).

The storage unit 1115 can store files, such as drivers, libraries and saved programs. The storage unit 1115 can store user data, e.g., user preferences and user programs. The computer system 1101 in some cases can include one or more additional data storage units that are external to the computer system 1101, such as located on a remote server that is in communication with the computer system 1101 through an intranet or the Internet.

The computer system 1101 can communicate with one or more remote computer systems through the network 1130. For instance, the computer system 1101 can communicate with a remote computer system of a user (e.g., operator). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 1101 via the network 1130.

Methods as described herein can be implemented by way of machine (e.g., computer processor) executable code stored on an electronic storage location of the computer system 1101, such as, for example, on the memory 1110 or electronic storage unit 1115. The machine executable or machine readable code can be provided in the form of software. During use, the code can be executed by the processor 1105. In some cases, the code can be retrieved from the storage unit 1115 and stored on the memory 1110 for ready access by the processor 1105. In some situations, the electronic storage unit 1115 can be precluded, and machine-executable instructions are stored on memory 1110.

The code can be pre-compiled and configured for use with a machine having a processor adapted to execute the code, or can be compiled during runtime. The code can be supplied in a programming language that can be selected to enable the code to execute in a pre-compiled or as-compiled fashion.

Aspects of the systems and methods provided herein, such as the computer system 201, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1101 can include or be in communication with an electronic display 235 that comprises a user interface (UI) 1140 for providing, for example, results of the assay, such as a summary of variant antibodies, or variant antigen-binding fragments thereof that bind to the target antigen. Examples of UIs include, without limitation, a graphical user interface (GUI) and web-based user interface.

Methods and systems of the present disclosure can be implemented by way of one or more algorithms. An algorithm can be implemented by way of software upon execution by the central processing unit 1105. The algorithm can, for example, contact a target antigen with a variant antibody, or variant antigen-binding fragment thereof, isolate the variant antibody, or variant antigen-binding fragment thereof, or identify the variant antibody, or variant antigen-binding fragment thereof, as described herein. In some embodiments, an algorithm can determine a relative dissociation constant for a variant antibody, or variant antigen-binding fragment thereof. The algorithm can further identify, based at least on the relative dissociation constant, the variant antibody, or variant antigen-binding fragment thereof, as binding specifically to a target antigen.

Devices, systems, compositions and methods of the present disclosure may be used for various applications, such as, for example, processing a single analyte (e.g., RNA, DNA, or protein) or multiple analytes (e.g., DNA and RNA, DNA and protein, RNA and protein, or RNA, DNA and protein) from a single cell. For example, a biological particle (e.g., a cell, nucleus of a cell, or cell bead) can be partitioned in a partition (e.g., droplet), and multiple analytes from the biological particle, can be processed for subsequent processing. The multiple analytes may be from the single biological particle (e.g., cell, nucleus of a cell, or cell bead). This may enable, for example, simultaneous proteomic, transcriptomic and genomic analysis of the cell.

All publications and patent applications mentioned in this disclosure are herein incorporated by reference to the same extent as if each individual publication or patent application was specifically and individually indicated to be incorporated by reference.

No admission is made that any reference cited herein constitutes prior art. The discussion of the references states what their authors assert, and the Applicant reserves the right to challenge the accuracy and pertinence of the cited documents. It will be clearly understood that, although a number of information sources, including scientific journal articles, patent documents, and textbooks, are referred to herein; this reference does not constitute an admission that any of these documents forms part of the common general knowledge in the art.

The discussion of the general methods given herein is intended for illustrative purposes only. Other alternative methods and alternatives will be apparent to those of skill in the art upon review of this disclosure, and are to be included within the spirit and purview of this application.

Additional embodiments are disclosed in further detail in the following examples, which are provided by way of illustration and are not in any way intended to limit the scope of this disclosure or the claims.

### EXAMPLES

### Example 1

### Error Prone PCR

Error prone PCR will be performed on purified antibody clone material, which may be from a construct (or a set of constructs) that include the coding sequence for the antibody or an antigen-binding fragment of the antibody or may be from a cDNA library. By amplifying the target sequences using Taq DNA polymerase in the presence of Mn, Mg, and K salts, with variable ratios of dNTPs, pools of product containing random mutations will be created. The high divalent cation environment of the error-prone PCR reaction leads to an increase in the frequency of improper base pairing events

These products can then be inserted into expression vectors and transfected into cells, e.g., B cells. The cells can then be screened for characteristics including antibody specificity and/or antibody affinity.

While particular alternatives of the present disclosure have been disclosed, it is to be understood that various modifications and combinations are possible and are contemplated within the true spirit and scope of the appended claims. There is no intention, therefore, of limitations to the exact abstract and disclosure herein presented.

1. A method of engineering an antigen-binding site of an antibody, or antigen-binding fragment thereof, to comprise an altered characteristic, the method comprising:
providing a nucleic acid sequence encoding a selected antibody, or selected antigen-binding fragment thereof,
wherein the selected antibody, or selected antigen-binding fragment thereof, binds a target antigen;
amplifying the nucleic acid sequence in an error-prone amplification reaction to produce a plurality of polynucleotides encoding variant antibodies, or variant antigen-binding fragments thereof;
expressing the plurality of variant antibodies, or variant antigen-binding fragments thereof, in a plurality of cells,
wherein a cell of the plurality of cells expresses a variant antibody, or variant antigen-binding fragment thereof, of the plurality of variant antibodies, or variant antigen-binding fragments thereof;
incubating the plurality of cells in a reaction mixture further comprising the target antigen,
wherein the target antigen is coupled to a reporter oligonucleotide;
partitioning the reaction mixture into a plurality of partitions, wherein a partition of the plurality of partitions comprises:
(i) a partitioned cell bound to the target antigen, and
(ii) a plurality of nucleic acid barcode molecules comprising a partition-specific barcode sequence;
in the partition, generating barcoded nucleic acid molecules, wherein the barcoded nucleic acid molecules comprise:
(i) a first barcoded nucleic acid molecule comprising a sequence of the reporter oligonucleotide or a reverse complement thereof and the partition-specific barcode sequence or reverse complement thereof, and
(ii) a second barcoded nucleic acid molecule comprising a nucleic acid sequence encoding the variant antibody, or variant antigen-binding fragment thereof, expressed by the cell or reverse complement thereof and the partition-specific barcode sequence or reverse complement thereof, and
identifying the variant antibody, or variant antigen-binding fragment thereof, as engineered to comprise the altered characteristic based on the generated first barcoded nucleic acid molecule.

2. A method of preparing a library of variant antibodies, or variant antigen-binding fragments thereof, comprising an altered characteristic or characteristics, the method comprising:
providing a nucleic acid sequence encoding a selected antibody, or selected antigen-binding fragment thereof,
wherein the selected antibody, or selected antigen-binding fragment thereof, binds a target antigen;
amplifying the nucleic acid sequence in an error-prone amplification reaction to produce a plurality of polynucleotides encoding variant antibodies, or variant antigen-binding fragments thereof;
expressing the plurality of variant antibodies, or variant antigen-binding fragments thereof, in a plurality of cells,
wherein a cell of the plurality of cells expresses a variant antibody, or variant antigen-binding fragment thereof, of the plurality of variant antibodies, or variant antigen-binding fragments thereof;
incubating the plurality of cells in a reaction mixture further comprising the target antigen,
wherein the target antigen is coupled to a reporter oligonucleotide;
partitioning the reaction mixture into a plurality of partitions, wherein a partition of the plurality of partitions comprises:
(i) a partitioned cell bound to the target antigen, and
(ii) a plurality of nucleic acid barcode molecules comprising a partition-specific barcode sequence;
in the partition, generating barcoded nucleic acid molecules, wherein the barcoded nucleic acid molecules comprise:
(i) a first barcoded nucleic acid molecule comprising a sequence of the reporter oligonucleotide or a reverse complement thereof and the partition-specific barcode sequence or reverse complement thereof, and
(ii) a second barcoded nucleic acid molecule comprising a nucleic acid sequence encoding the variant antibody, or variant antigen-binding fragment thereof, expressed by the cell or reverse complement thereof and the partition-specific barcode sequence or reverse complement thereof; and
identifying the variant antibody, or variant antigen-binding fragment thereof, for inclusion in the library based on the generated first barcoded nucleic acid molecule.

3. A method of engineering an antigen-binding site of an antibody, or antigen-binding fragment thereof, to comprise an altered characteristic, the method comprising:
providing a nucleic acid sequence encoding a selected antibody, or selected antigen-binding fragment thereof,
wherein the selected antibody, or selected antigen-binding fragment thereof, binds a target antigen;
amplifying the nucleic acid sequence in an error-prone amplification reaction to produce a plurality of polynucleotides encoding variant antibodies, or variant antigen-binding fragments thereof;
expressing the plurality of variant antibodies, or variant antigen-binding fragments thereof, in a plurality of cells,
wherein a cell of the plurality of cells expresses a variant antibody, or variant antigen-binding fragment thereof, of the plurality of variant antibodies, or variant antigen-binding fragments thereof;
incubating the plurality of cells in a reaction mixture further comprising the target antigen,
wherein the target antigen is coupled to a reporter oligonucleotide;
partitioning the reaction mixture into a plurality of partitions, wherein a partition of the plurality of partitions comprises:
(i) a partitioned cell bound to the target antigen, and
(ii) a plurality of nucleic acid barcode molecules comprising a partition-specific barcode sequence;
generating barcoded nucleic acid molecules, wherein the barcoded nucleic acid molecules comprise:
(i) a first barcoded nucleic acid molecule comprising a sequence of the reporter oligonucleotide or a reverse complement thereof and the partition-specific barcode sequence or reverse complement thereof, and
(ii) a second barcoded nucleic acid molecule comprising a nucleic acid sequence encoding the variant antibody, or variant antigen-binding fragment thereof, expressed by the cell or reverse complement thereof and the partition-specific barcode sequence or reverse complement thereof, and
identifying the variant antibody, or variant antigen-binding fragment thereof, as engineered to comprise the altered characteristic based on the generated first barcoded nucleic acid molecule.

4. A method of preparing a library of variant antibodies, or variant antigen-binding fragments thereof, comprising an altered characteristic or characteristics, the method comprising:
providing a nucleic acid sequence encoding a selected antibody, or selected antigen-binding fragment thereof,
wherein the selected antibody, or selected antigen-binding fragment thereof, binds a target antigen;
amplifying the nucleic acid sequence in an error-prone amplification reaction to produce a plurality of polynucleotides encoding variant antibodies, or variant antigen-binding fragments thereof;
expressing the plurality of variant antibodies, or variant antigen-binding fragments thereof, in a plurality of cells,
wherein a cell of the plurality of cells expresses a variant antibody, or variant antigen-binding fragment thereof, of the plurality of variant antibodies, or variant antigen-binding fragments thereof;
incubating the plurality of cells in a reaction mixture further comprising the target antigen,
wherein the target antigen is coupled to a reporter oligonucleotide;
partitioning the reaction mixture into a plurality of partitions, wherein a partition of the plurality of partitions comprises:
(i) a partitioned cell bound to the target antigen, and
(ii) a plurality of nucleic acid barcode molecules comprising a partition-specific barcode sequence;
generating barcoded nucleic acid molecules, wherein the barcoded nucleic acid molecules comprise:
(i) a first barcoded nucleic acid molecule comprising a sequence of the reporter oligonucleotide or a reverse complement thereof and the partition-specific barcode sequence or reverse complement thereof, and
(ii) a second barcoded nucleic acid molecule comprising a nucleic acid sequence encoding the variant antibody, or variant antigen-binding fragment thereof, expressed by the cell or reverse complement thereof and the partition-specific barcode sequence or reverse complement thereof, and
identifying the variant antibody, or variant antigen-binding fragment thereof, for inclusion in the library based on the generated first barcoded nucleic acid molecule.

5. The method of any one of claims 1-4, wherein the provided nucleic acid sequence encoding the selected antibody, or selected antigen-binding fragment thereof, is of a plurality of nucleic acid sequences encoding a plurality of selected antibodies, or selected antigen-binding fragments thereof, that bind the target antigen.

6. The method of any preceding claim, wherein the selected antibody, or selected antigen-binding fragment thereof, comprises a human antibody, or antigen-binding fragment thereof.

7. The method of any preceding claim, wherein the provided nucleic acid sequence encoding the selected antibody, or selected antigen-binding fragment thereof, is derived from nucleic acids of a cell of a human donor or a combination of cells from multiple human donors.

8. The method of claim 7, wherein the cell or cells are from a blood sample, a peripheral blood mononuclear cell sample of the human donor, and wherein the cell or cells optionally is a B cell.

9. The method any of claims 7-8, wherein the donor or donors have been exposed to the target antigen, is suspected of having been exposed to the target antigen, or is resistant to a disease associated with the target antigen.

10. The method of any of claims 1-4, wherein the provided nucleic acid sequence encoding the selected antibody, or selected antigen-binding fragment thereof, is derived from nucleic acids of a cell of a mouse, wherein the mouse is a transgenic mouse, wherein optionally the transgenic mouse expresses human immunoglobulin genes, wherein optionally the cell of the mouse is from a blood sample of the mouse, wherein optionally the cell is a B cell, wherein optionally the mouse had been exposed to the target antigen.

11. The method of claim 5, wherein the plurality of nucleic acid sequences encoding the plurality of selected antibodies, or selected antigen-binding fragments thereof, are derived from nucleic acids of a combination of cells from mice, wherein the cells from the mice are from blood samples of the mice, wherein optionally the cells are B cells, wherein optionally the mice had been exposed to the target antigen.

12. The method of any preceding claim, wherein the reporter oligonucleotide comprises a reporter barcode sequence and a capture handle sequence.

13. The method of any preceding claim, wherein a first nucleic acid barcode molecule of the plurality of barcode molecules further comprises a capture sequence configured to couple to the capture handle sequence, and wherein a second nucleic acid barcode molecule of the plurality of nucleic acid barcode molecules further comprises a capture sequence configured to couple to an mRNA or DNA analyte.

14. The method of claim 13, wherein the capture handle sequence is configured to couple to the capture sequence by complementary base pairing.

15. The method of claim 14, wherein the capture sequence is configured to couple to the mRNA analyte, and wherein the capture sequence configured to couple to the mRNA analyte comprises a polyT sequence.

16. The method of any of claims 1-12, wherein a first nucleic acid barcode molecule of the plurality of barcode molecules further comprises a capture sequence configured to couple to the capture handle sequence, and wherein a second nucleic acid barcode molecule of the plurality of nucleic acid barcode molecules further comprises a capture sequence configured to couple to non-templated nucleotides appended to a cDNA reverse transcribed from an mRNA analyte, wherein optionally the second nucleic acid barcode molecule further comprises a template switch oligonucleotide (TSO).

17. The method of claim 16, wherein the mRNA analyte is reverse transcribed to the cDNA utilizing a primer comprising a polyT sequence.

18. The method of claim 16 or 17, wherein the non-templated nucleotides appended to the cDNA comprise a cytosine, wherein optionally the capture sequence configured to couple to the cDNA comprise a guanine, optionally wherein. coupling of the capture sequence to the non-templated cytosine extends reverse transcription of the cDNA into the second nucleic acid barcode to generate the second barcoded nucleic molecule.

19. The method of any preceding claim, wherein the target antigen coupled to the reporter oligonucleotide comprises a detectable label.

20. The method of claim 19, wherein the detectable label is fluorescent or magnetic.

21. The method of any preceding claim, wherein the first and/or second barcoded nucleic molecule further comprises a UMI sequence.

22. The method of any preceding claim, further comprising determining sequences of the first and the second barcoded nucleic acid molecule, wherein optionally the variant antibody or variant antigen binding fragment thereof is identified based on the determined sequence of the second barcoded nucleic acid molecule, wherein optionally the determined sequence comprises a nucleotide sequence, wherein optionally the determined sequence comprises an amino acid sequence encoded by the nucleotide sequence.

23. The method of any preceding claim, further comprising assessing affinity of the variant antibody, or variant antigen binding fragment thereof, based on the generated quantity of the first barcoded nucleic acid molecule.

24. The method of any preceding claim, wherein the reaction mixture further comprises an antigen not bound by the selected antibody, or selected antigen-binding fragment thereof,
wherein the antigen not bound by the selected antibody, or selected antigen-binding fragment thereof, is coupled to a second reporter oligonucleotide.

25. The method of claim 24, wherein the antigen not bound by the selected antibody, or selected antigen-binding fragment thereof, is a negative control antigen, wherein optionally the negative control antigen is human serum albumin.

26. The method of any preceding claim, wherein a second partition of the plurality of partitions comprises (i) a second cell expressing the selected antibody, or selected antigen-binding fragment thereof, bound to the target antigen, and (ii) the plurality of nucleic acid barcode molecules comprising the partition-specific barcode sequence.

27. The method of any preceding claim, wherein the cell is a mammalian cell, an insect cell or a yeast cell.

28. The method of any preceding claim, wherein the cell is a mammalian cell, wherein optionally the mammalian cell is a human cell, a Chinese hamster ovary cell, a mouse cell, a baby hamster kidney cell, a VERO cell, an NS0 or an HEK293 cell, wherein optionally the mammalian cell is the human cell.

29. A method of engineering an antigen-binding site of an antibody, or antigen-binding fragment thereof, to comprise an altered characteristic, the method comprising:
providing a nucleic acid sequence encoding a selected antibody derived from nucleic acids of a cell of a human donor, or selected antigen-binding fragment thereof,
wherein the selected human antibody, or selected antigen-binding fragment thereof, binds a target antigen;
amplifying the nucleic acid sequence in an error-prone amplification reaction to produce a plurality of polynucleotides encoding variant antibodies, or variant antigen-binding fragments thereof;
expressing the plurality of variant antibodies, or variant antigen-binding fragments thereof, in a plurality of mammalian cells, wherein a mammalian cell of the plurality of mammalian cells expresses a variant antibody, or variant antigen-binding fragment thereof of the plurality of variant antibodies or variant antigen-binding fragments thereof; and
identifying the variant antibody, or variant antigen-binding fragment thereof, as engineered to comprise the altered characteristic.

30. A method of preparing a library of variant antibodies, or variant antigen-binding fragments thereof, comprising an altered characteristic or characteristics, the method comprising:
providing a nucleic acid sequence encoding a selected antibody derived from nucleic acids of a cell of a human donor, or selected antigen-binding fragment thereof,
wherein the selected human antibody, or selected antigen-binding fragment thereof, binds a target antigen;
amplifying the nucleic acid sequence in an error-prone amplification reaction to produce a plurality of polynucleotides encoding variant antibodies, or variant antigen-binding fragments thereof;
expressing the plurality of variant antibodies, or variant antigen-binding fragments thereof, in a plurality of mammalian cells, wherein a mammalian cell of the plurality of mammalian cells expresses a variant antibody, or variant antigen-binding fragment thereof, of the plurality of variant antibodies or variant antigen-binding fragments thereof, and
identifying the variant antibody, or variant antigen-binding fragment thereof, for inclusion in the library if it comprises an altered characteristic or characteristics.

31. The method of claim 29 or 30, wherein the provided nucleic acid sequence encoding the selected antibody derived from nucleic acids of a cell of a human donor, or selected antigen-binding fragment thereof, is of a plurality of nucleic acid sequences encoding a plurality of selected antibodies derived from nucleic acids of cells of human donors, or selected antigen-binding fragments thereof, that bind the target antigen.

32. The method of claim 29 or 30, wherein the cell of the human donor is from a blood sample or a peripheral blood mononuclear cell sample of the human donor, optionally wherein the cell is a B cell.

33. The method of claim 32, wherein the cell of the human donors, or selected antigen-binding fragments thereof, of the humans donors are of a combination of blood samples or a peripheral blood mononuclear cell samples of the human donors, wherein optionally the cell is a B cells, wherein optionally the donor was exposed to the target antigen, or suspected of having been exposed to the target antigen, or resistant to a disease associated with the target antigen, wherein optionally the donors were exposed to the target antigen, or were suspected of having been exposed to the target antigen, or are resistant to a disease associated with the target antigen.

34. The method of any of claims 31-33, further comprising expressing the selected human antibody, or the selected antigen-binding fragment thereof, in a second of the plurality of mammalian cells.

35. The method of claim 34, further comprising comparing a characteristic of the selected human antibody, or the selected antigen-binding fragment thereof, to the characteristic of the variant human antibody, or variant antigen-binding fragment thereof.

36. The method of claim 35, further comprising determining the characteristic of the selected human antibody, or the selected antigen-binding fragment thereof, is altered relative to the characteristic of the variant human antibody, or variant antigen-binding fragment thereof.

37. The method of any of claims 31-36, further comprising determining a sequence of the variant antibody, or variant antigen-binding fragment thereof, wherein optionally the determined sequence comprises a nucleotide sequence, wherein optionally the determined sequence comprises an amino acid sequence encoded by the nucleotide sequence.

38. The method of any of claims 31-37, wherein the variant antibody, or variant antigen-binding fragment thereof is not expressed in a non-mammalian cell prior to the expressing.

39. The method of any preceding claim, wherein the target antigen is a pathogen selected from a virus, a bacterial cell or a parasite.

40. The method of any of claims 1-38, wherein the target antigen is selected from a virus-like particle, lipoparticle, a cytokine or a fragment thereof, an autoantigen or a fragment thereof.

41. The method of any of claims 1-38, wherein the target antigen is a tumor-associated antigen or a fragment thereof, wherein optionally the tumor-associated antigen is a growth factor of a growth factor receptor or a fragment thereof.

42. A system for engineering an antigen-binding site of an antibody, or antigen-binding fragment thereof, to comprise an improved characteristic comprising:
(i) reagents for performing error-prone amplification,
(ii) a target antigen coupled to a reporter oligonucleotide,
(iii) a plurality of nucleic acid barcode molecules comprising a partition-specific barcode sequence, and
(iv) reagents for generating a plurality barcoded nucleic acid molecules formed by complementary base pairing of (a) a capture sequence of the plurality of nucleic acid barcode molecules and (b) a capture handle sequence of the first reporter oligonucleotide and/or a capture handle sequence of a polynucleotide encoding an engineered variant of the antibody or antigen-binding fragment thereof.

43. The system of claim 42, further comprising: (iv) reagents for generating a plurality barcoded nucleic acid molecules formed by complementary base pairing of (a) a capture sequence of the plurality of nucleic acid barcode molecules and (b) a capture handle sequence of the first reporter oligonucleotide and/or a capture handle sequence of a polynucleotide encoding an engineered variant of the antibody or antigen-binding fragment thereof

44. The system of claim 43, further comprising a sequencer or sequencing system.

45. The system of any of claims 42-44, further comprising reagents for determining affinity of the engineered antibody, or antigen-binding fragment thereof, from a second barcoded nucleic acid molecule of the plurality of barcoded nucleic acid molecules, formed from complementary base pairing of the capture sequence of the plurality of nucleic acid barcode molecules and the capture handle sequence of the first reporter oligonucleotide.

46. The system of any of claims 42-45, further comprising an analysis engine.

47. The system of any of claims 42-46, further comprising a network.

48. The system of any of claims 42-47, further comprising a microfluidic device for generating a partition comprising:
(ii) the target antigen coupled to the reporter oligonucleotide,
(iii) the plurality of nucleic acid barcode molecules comprising the partition-specific barcode sequence,
(iv) the reagents for generating the plurality barcoded nucleic acid molecules; and
(v) a cell expressing the antibody, or antigen-binding fragment thereof, comprising an engineered antigen-binding site, the antigen-binding site having been engineered by the (i) reagents for performing error-prone amplification.

49. The method of claim 30, further comprising steps wherein:
(i) the cell of the human donor is identified as expressing the selected antibody, or selected antigen-binding fragment thereof, and
(ii) the nucleic acid sequence encoding the selected antibody, or antigen-binding fragment thereof, is prepared for the providing,
the steps comprising:
incubating the cell of the human donor with the target antigen, wherein the target antigen is coupled to a reporter oligonucleotide;
partitioning the cell of the human donor bound to the target antigen in a partition further comprising a plurality of nucleic acid barcode molecules comprising a partition-specific barcode sequence;
in the partition, generating barcoded nucleic acid molecules, wherein the barcoded nucleic acid molecules comprise:
(i) a first barcoded nucleic acid molecule comprising the sequence of the reporter oligonucleotide or a reverse complement thereof and the partition-specific barcode sequence or reverse complement thereof,
wherein detection of the first barcoded nucleic acid molecule identifies the cell of the human donor as expressing the selected antibody, or the selected antigen-binding fragment thereof, and
(ii) a second barcoded nucleic acid molecule comprising the nucleic acid sequence encoding the selected antibody, or selected antigen-binding fragment thereof, for the providing.

50. The method of claim 49, further comprising steps wherein:
(i) the cells of the human donors are identified as expressing the plurality of selected antibodies or selected antigen-binding fragments thereof, and
(ii) the nucleic acid sequences encoding the selected antibodies, or antigen-binding fragments thereof, are prepared for the providing,
the steps comprising:
incubating the cells of the human donors in a reaction mixture comprising the target antigen,
wherein the target antigen is coupled to a reporter oligonucleotide;
partitioning the reaction mixture into a plurality of partitions, wherein a partition of the plurality of partitions comprises:
(i) a partitioned cell of the human donor bound to the target antigen, and
(ii) a plurality of nucleic acid barcode molecules comprising a partition-specific barcode sequence;
in the partition, generating barcoded nucleic acid molecules, wherein the barcoded nucleic acid molecules comprise:
(i) a first barcoded nucleic acid molecule comprising the sequence of the reporter oligonucleotide or a reverse complement thereof and the partition-specific barcode sequence or reverse complement thereof,
wherein detection of the first barcoded nucleic acid molecule identifies the partitioned cell of the human donor as expressing the selected antibody, or the selected antigen-binding fragment thereof, and
(ii) a second barcoded nucleic acid molecule comprising the nucleic acid sequence encoding the selected antibody, or selected antigen-binding fragment thereof, for the providing.

51. The method of any one of claims 8-30, further comprising steps wherein:
(i) the cell of the human donor, the cells of the human donors, the cell of the mouse, the cells from the mice are identified as expressing the selected antibody, or selected antigen-binding fragment thereof, and
(ii) the nucleic acid sequence encoding the selected antibody, or antigen-binding fragment thereof, is prepared for the providing,
the steps comprising:
incubating the cell of the human donor, the cells of the human donors, the cell of the mouse, the cells from the mice with the target antigen, wherein the target antigen is coupled to a reporter oligonucleotide;
partitioning the cell of the human donor, the cells of the human donors, the cell of the mouse, the cells from the mice bound to the target antigen in a partition further comprising a plurality of nucleic acid barcode molecules comprising a partition-specific barcode sequence;
in the partition, generating barcoded nucleic acid molecules, wherein the barcoded nucleic acid molecules comprise:
(i) a first barcoded nucleic acid molecule comprising the sequence of the reporter oligonucleotide or a reverse complement thereof and the partition-specific barcode sequence or reverse complement thereof,
wherein detection of the first barcoded nucleic acid molecule identifies the cell of the human donor, the cells of the human donors, the cell of the mouse, the cells from the mice as expressing the selected antibody, or the selected antigen-binding fragment thereof, and
(ii) a second barcoded nucleic acid molecule comprising the nucleic acid sequence encoding the selected antibody, or selected antigen-binding fragment thereof, for the providing.

52. The method of claim 51, further comprising steps wherein:
(i) the cell of the human donor, the cells of the human donors, the cell of the mouse, the cells from the mice are identified as expressing the plurality of selected antibodies or selected antigen-binding fragments thereof, and
(ii) the nucleic acid sequences encoding the selected antibodies, or antigen-binding fragments thereof, are prepared for the providing,
the steps comprising:
incubating the cell of the human donor, the cells of the human donors, the cell of the mouse, the cells from the mice in a reaction mixture comprising the target antigen,
wherein the target antigen is coupled to a reporter oligonucleotide;
partitioning the reaction mixture into a plurality of partitions, wherein a partition of the plurality of partitions comprises:
(i) a partitioned cell of the human donor, the cells of the human donors, the cell of the mouse, the cells from the mice bound to the target antigen, and
(ii) a plurality of nucleic acid barcode molecules comprising a partition-specific barcode sequence;
in the partition, generating barcoded nucleic acid molecules, wherein the barcoded nucleic acid molecules comprise:
(i) a first barcoded nucleic acid molecule comprising the sequence of the reporter oligonucleotide or a reverse complement thereof and the partition-specific barcode sequence or reverse complement thereof,
wherein detection of the first barcoded nucleic acid molecule identifies the partitioned the cell of the human donor, the cells of the human donors, the cell of the mouse, the cells from the mice as expressing the selected antibody, or the selected antigen-binding fragment thereof, and
(ii) a second barcoded nucleic acid molecule comprising the nucleic acid sequence encoding the selected antibody, or selected antigen-binding fragment thereof, for the providing.

53. The method of any one of claims 49-52, wherein the reporter oligonucleotide comprises a reporter barcode sequence and a capture handle sequence.

54. The method of any of claims 49-53, wherein a first nucleic acid barcode molecule of the plurality of barcode molecules further comprises a capture sequence configured to couple to the capture handle sequence, and wherein a second nucleic acid barcode molecule of the plurality of nucleic acid barcode molecules further comprises a capture sequence configured to couple to an mRNA or DNA analyte.

55. The method of claim 54, wherein the capture handle sequence is configured to couple to the capture sequence by complementary base pairing.

56. The method of claim 55, wherein the capture sequence is configured to couple to the mRNA analyte, and wherein the capture sequence configured to couple to the mRNA analyte comprises a polyT sequence.

57. The method of any of claims 49-56, wherein a first nucleic acid barcode molecule of the plurality of barcode molecules further comprises a capture sequence configured to couple to the capture handle sequence, and wherein a second nucleic acid barcode molecule of the plurality of nucleic acid barcode molecules further comprises a capture sequence configured to couple to non-templated nucleotides appended to a cDNA reverse transcribed from an mRNA analyte, wherein optionally the second nucleic acid barcode molecule further comprises a template switch oligonucleotide (TSO).

58. The method of claim 57, wherein the mRNA analyte is reverse transcribed to the cDNA utilizing a primer comprising a polyT sequence.

59. The method of claim 57 or 58, wherein the non-templated nucleotides appended to the cDNA comprise a cytosine, wherein optionally the capture sequence configured to couple to the cDNA comprise a guanine wherein optionally coupling of the capture sequence to the non-templated cytosine extends reverse transcription of the cDNA into the second nucleic acid barcode to generate the second barcoded nucleic molecule.

60. The method of any of claims 49-59, wherein the target antigen coupled to the reporter oligonucleotide comprises a detectable label.

61. The method of claim 60, wherein the detectable label is fluorescent or magnetic.

62. The method of any preceding claim, wherein the first and/or second barcoded nucleic molecule further comprises a UMI sequence.

63. The method of any of claims 49-62, further comprising determining sequences of the first and the second barcoded nucleic acid molecule.

64. The method of any of claims 49-63, wherein the target antigen is a pathogen, a virus, a bacterial cell or a parasite, a virus-like particle or lipoparticle, a cytokine or a fragment thereof, a tumor-associated antigen or a fragment thereof, a growth factor of a growth factor receptor or a fragment thereof, an autoantigen or a fragment thereof.

65. The method of any of claims 1-41 or 49-64, wherein the error-prone amplification comprises rolling circle amplification or polymerase chain reaction.

66. The method of any of claims 1-41 or 49-65, wherein the error-prone amplification introduces errors via a low fidelity polymerase enzyme.

67. The method of any of claims 1-41 or 49-66, wherein the error-prone amplification introduces errors via increasing MgCl₂ concentration, addition of MnCl₂, or both increasing MgCl₂ concentration and addition of MnCl₂ in the reaction.

68. The method of any of claims 1-41 or 49-67, wherein the error-prone amplification introduces errors via an imbalance in ratio in deoxynucleoside triphosphate concentrations in the reaction.

69. The method of any of claims 1-41 or 49-68, wherein the variant antibody, or variant antigen-binding fragment thereof, comprises at least one, at least three, or at least five amino acid substitution relative to the selected antibody or antigen binding fragment thereof.

70. The method of any of claims 1-41 or 49-69, wherein the altered characteristic comprises altered affinity, an altered association constant, an altered dissociation constant, altered specificity for the target antigen, wherein optionally when the altered characteristic is altered affinity it is a higher affinity.

71. The method of claim 70, further comprising subjecting the identified variant antibody, or identified variant antigen-binding fragment thereof, to an activity assay.

72. The method of claim 71, wherein identified variant antibody, or identified variant antigen-binding fragment thereof, further comprises an altered activity.

73. The method of any of claims 49-72, wherein the altered characteristic comprises an altered activity.

74. The method of claim 73, wherein the altered activity comprises one or more of an alteration in neutralization, inflammation, tumor growth or vascularization, or immune stimulation.

75. The method of any preceding claim, wherein the antibody is a secreted antibody or an antigen binding fragment thereof.

76. The method of claim 75, wherein the secreted antibody is coupled to the cell via a capture agent.

77. The method of claim 76, wherein the capture agent is configured to couple to a cell surface molecule.

78. The method of claim 77, wherein the cell surface molecule is a cell surface protein.

79. The method of any one of claims 75-78, wherein the capture agent is configured to couple to the secreted antibody or antigen fragment thereof.

80. The method of claim 79, wherein the capture agent is configured to couple to both the cell surface protein and said secreted antibody or antigen binding fragment thereof.
